(19) United States
(12) Patent Application Publication
(10) Pub. No.: US 2026/0112474 A1
(43) Pub. Date: Apr. 23, 2026

(54) SYSTEM FOR VISUALIZING NUTRITIONAL INFORMATION AND PROVIDING RECOMMENDATIONS BASED ON MULTIMODAL USER INPUTS AND INTERACTIVE VISUALIZATIONS

(71) Applicant: Abbott Diabetes Care Inc., Alameda, CA (US)

(72) Inventors: Saranpreet Singh NAGRA, Alameda, CA (US); Gary A. HAYTER, Oakland, CA (US); Timothy Christian DUNN, San Francisco, CA (US); Byron Paul OLSON, II, Sioux Falls, SD (US); Douglas Paul RICHES, Burlington Ontario (CA); Prarthana PRASHANTH, Foster City, CA (US)

(21) Appl. No.: 19/362,095

(22) Filed: Oct. 17, 2025

Related U.S. Application Data
(60) Provisional application No. 63/709,371, filed on Oct. 18, 2024.
(60) Provisional application No. 63/821,324, filed on Jun. 10, 2025.

(51) Int. Cl. G16H 20/60; G16H 10/60; G16H 40/67

(52) U.S. Cl. CPC G16H 20/60; G16H 10/60; G16H 40/67

(57) ABSTRACT

Disclosed herein is a multimodal meal model configured to receive a variety of inputs associated with one or more meals, the inputs including image data, textual data, video data, and audio data. Inputs may be combined into a predefined prompt template that includes placeholders defined for certain types of inputs and includes instructions for how the output from the model should be structured. The meal visualization model is configured to generate dynamically annotated and interactive graphical user interfaces based on the inputs including interfaces that provide interactive graphical elements for manipulating meal components identified within the inputs. The model is also configured to provide personalized recommendations based on the inputs and user data, such as user glucose information and medical history. The interactive user interfaces may also be configured to display the personalized recommendations, which may also include interactive elements that enable manipulation of the recommendations by the user.

### CROSS-REFERENCE TO RELATED APPLICATIONS

This application claims priority to U.S. provisional application 63/709,371, filed on Oct. 18, 2024 and U.S. provisional application 63/821,324, filed on Jun. 10, 2025, the entirety of which are hereby incorporated by reference.

### FIELD OF THE INVENTION

The subject matter of this disclosure generally relates to systems, devices, and methods for processing multimodal inputs to extract meal information (e.g., metadata) for improved efficiency and accuracy in meal logging and glucose projection, and for generating dynamically annotated and interactive visual representations of the multimodal inputs.

### BACKGROUND

The increased prevalence of Type-Two diabetes and metabolic syndrome over the past few decades has been attributed to changing diet and activity levels. The consumption of readily available high glycemic index (GI) foods causes rapid increases in blood glucose and insulin levels, which are positively associated with weight gain and obesity. These conditions further increase the risk of developing various diseases.

Current meal tracking software applications face significant challenges in accurately facilitating dietary monitoring. The requirement for manual input of meals and ingredients results in decreased user engagement and usage. This manual logging leads to incomplete or inaccurate dietary data, making it difficult to provide meaningful insights into the physiological impact of food choices. The lack of immediate visualization tools for displaying the effects of food intake contributes to misconceptions about portion sizes and the healthiness of various foods. Additionally, the absence of integrated activity tracking further complicates the understanding of the necessary duration and intensity of physical activity required to maintain good health. External influences, such as advertisements, habits, peer pressure, food preferences, and generalized recommendations, exacerbate these tracking and interpretation issues.

Glucose monitoring systems offer a way to track and understand individuals' physiological responses to food consumption. High glucose levels, driven by food intake, can provide valuable data on carbohydrate consumption and physiological responses to meals. However, one challenge lies in using this data meaningfully to enable accurate recommended actions. The need for clinical and personal understanding of meal selection data and its impact on glucose excursions, such as hyperglycemia episodes, highlights the technological gaps in current systems.

Previous attempts at implementing meal tracking software and correlating it with analyte data suffer from several technological deficiencies. The dependency on manual meal logging introduces error potential and inaccurate recommended courses of action. Moreover, the reliance on discrete blood glucose measurements, often requiring inconvenient and uncomfortable finger stick tests, limits the number of data points available for analysis. This limitation makes it difficult to accurately determine glycemic responses to meals. For instance, measurements taken before or after the peak glycemic response fail to provide a comprehensive view, hindering meaningful meal comparisons. Additionally, the lack of automated meal event detection in analyte data and inability to make adjustments to the detected meal can result in inaccurate meal detection, and reduces the overall efficacy of these systems and, by extension, the efficacy of the recommendations generated from the detected meals.

Moreover, when a user first starts using an insulin dose recommendation system for meals, the system typically will not have access to any meal history, so the initial dose recommendations cannot be tailored based on the meals until the meal history becomes populated with user meals over time. This is a start-up problem found in insulin dose recommendation systems.

### BRIEF SUMMARY OF THE INVENTION

Provided herein are example embodiments of systems, devices, and methods (especially computer-implemented methods) for processing multimodal information such as free text, images/photographs, audio recordings, and videos, and potentially in combination with user health information, to extract and process meal information (e.g., metadata) into dynamic graphical user interfaces for presenting outputs of a meal visualization model. The graphical user interfaces include annotations and interactive elements that enable users to manipulate aspects of the meal to view potential impacts of meals on user health, such as the predicted glucose impact in response to consuming the meal. The meal visualization system that includes a meal visualization model described herein enables identification of meals based on inputs including multimodal information and user information for minimizing user interaction within the identification process. The multimodal meal visualization system may be in communication with one or more continuous analyte sensors to generate visualizations representing potential impact of the identified meals on analyte levels, allowing for display of data that highlights which meals or meal components significantly affect these levels. In some examples, the multimodal meal visualization system may have access to analyte data (e.g., not directly receive the analyte data from one or more continuous analyte sensors. This information can be systematically organized and categorized based on criteria preselected by the individual or established through consultation with a medical professional, ensuring personalized and actionable insights.

The meal visualization system provides technical improvements for multimodal input processing, advanced meal detection, glucose impact predictions, and customized and dynamically selected visual elements. The meal visualization system is capable of accepting diverse input types and combinations (e.g., meal images, text prompts, medical information including medical history, historical glucose information, user preferences), processing the input types to detect the input content and intent, combining detected input content with relevant input types such as historical user information to calculate a personalized glucose impact, and dynamically selecting and rendering visualization elements based on the multimodal inputs and personalized glucose impacts.

In some aspects, the meal visualization system may be implemented as a combination of a meal visualization model, user medical information devices (e.g., a continuous glucose monitoring system, a user electronic health record) and a visualization or display device. The meal visualization model facilitates secure communications for receiving the user medical information, such as from continuous glucose sensors, pumps, wearable health trackers, and databases, to acquire real-time physiological data and historical data.

The meal visualization system may utilize the data collected from these medical devices or an electronic health record as one or more set of inputs for the meal visualization model uses to provide personalized recommendations. Another set of inputs may include additional user-provided information related to dietary intake, user preferences (e.g. keto, high protein, paleo), and user dietary restrictions (e.g., allergies, vegan, vegetarian, lactose intolerant, gluten intolerant). In some aspects, the meal visualization model is configured to receive the additional user-provided information as multi-modal inputs, which may include various forms of media such as images, audio recordings, videos, or textual descriptions. In some aspects, the additional user-provided information may be provided via interactive visual interface components provided by the visualization device. The meal visualization model therefore is implemented as an engine for ingesting inputs provided from two or more different systems (e.g., medical devices, the visualization device, electronic medical record systems) and providing, based on those inputs, personalized recommendations in the form of interactive annotated visual interface components.

In some aspects, upon receiving these inputs, the meal visualization model is configured to generate an interactive visualization of the user-provided dietary information based on user medical data and user-provided dietary information. This step can include detection of meal components within the user-provided dietary information and customizing the visualization based on the detected meal components and the user's medical data.

In some aspects, the meal visualization model may be implemented as a generative artificial intelligence meal visualization model that receives, as inputs, the multimodal information. In many embodiments, the source of user medical data may include an analyte monitoring system, and meal-related analyte responses collected by the analyte monitoring system, such as an in vivo analyte monitoring system, can be also utilized as an input to the meal visualization model. Additionally, by integrating a generative model with the analyte monitoring system, the multimodal technology can go beyond conventional trend analysis. The generative model can provide personalized meal-related information, such as suggesting dietary adjustments, predicting potential glucose spikes based on specific meals, or even generating meal plans that optimize or reduce the individual's glycemic response. This connection between the analyte monitoring system and the generative AI model enables a more dynamic and intelligent approach to generating personalized graphical user interfaces with personalized, real-time, data-driven recommendations that adapt to a user's specific meals and physiological responses.

In some aspects, an interactive visualization is generated based on outputs of the meal visualization model. The interactive visualization allows the user to modify specific attributes of the detected meal components, such as portion size, ingredients, identification, or nutritional content. The interactive visualization may also configured to receive additional unstructured text, such as user queries, and perform natural language processing. User modifications and any additional information such as unstructured text may then be reintroduced into the model as feedback inputs, allowing for refinement of the visualization and improving the accuracy of generated recommendations. This iterative feedback process enables the meal visualization model to improve its meal detection capabilities and become more tailored to the user's unique physiological responses over time, enhancing the relevance of future recommendations.

In some aspects, the data flow within meal visualization system includes transmission of physiological data to the meal visualization model, establishing a dataset for analysis. This data can be continuously updated to reflect the user's current health status, providing the meal visualization model with dynamic and up-to-date datasets as part of predicting the impact of meals on a user's glucose levels. User-provided dietary information may also be fed to meal visualization model as another set of inputs through an interface capable of processing multi-modal formats. The meal visualization model can be configured to employ image recognition, natural language processing, auditory processing, and other data analysis techniques (e.g., historical analysis) to extract relevant information from these media inputs, such as identifying the type of meal, identifying meal components of the meal, estimating portion sizes, and determining nutritional values. The meal visualization model can ingest these heterogeneous inputs to detect the meal components of the provided dietary information, predict the potential impact of the detected meal components on the user's health, and generating an interactive visualization for the detected meal components, the potential impact, as well as an additional interface for enabling further adjustments to be provided to the meal visualization model.

The interactive visualization is configured to receive adjustments to various food parameters, such as increasing or decreasing portion sizes or substituting ingredients, or providing additional user-information, such as user queries, additional multi-modal inputs (e.g., additional images, text, video, or audio). These adjustments may be fed back, as part of a feedback loop, to the meal visualization model, which can dynamically recalculate the predicted health impacts and update the visualization in real time. This recalculation can consider both the immediate nutritional impact of the adjustments and the long-term effects on the user's health, such as changes in predicted blood glucose levels or overall caloric balance. This feedback loop provides improvements to the meal visualization system so as to provide more accurate recommendations for user actions regarding their dietary habits and their influence on health outcomes. Additionally, the system can store historical data, enabling the meal visualization model to track changes over time and improve the accuracy and effectiveness of both meal component detection and recommendations, which helps in identifying long-term trends and making further informed decisions.

In some aspects, the meal visualization system integrates external data sources for additional inputs to the meal visualization model. These external data sources include weather conditions, location information (e.g., restaurants), activity levels (e.g., steps, pushes, exercise information such as type, intensity, duration), electronic health records (e.g., HbA1C values, comorbidities, medication information), external databases (e.g., food database, recipe database, nutrition database), or other data sources relevant to determining potential glycemic impacts for a user, to provide additional input for improving the accuracy of meal component detection and recommendations.

The disclosure includes any combination of the aforementioned aspects. As should be appreciated, the aforementioned aspects reflect different elements of a meal visualization system that are compatible with one another and may be combined in any combinations. In one embodiment, all of the aspects may be combined. In other embodiments, combinations of two or more aspects may be combined.

Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS/FIGURES

The accompanying drawings, which are incorporated herein and form a part of the specification, illustrate the present disclosure and, together with the description, further serve to explain the principles thereof and to enable a person skilled in the pertinent art to make and use the same.

FIG. 1A is a system overview of a sensor applicator, data receiving device, monitoring system, network, and remote system.

FIG. 1B is a diagram illustrating an operating environment of an example analyte monitoring system for use with the techniques described herein.

FIG. 2A is a block diagram depicting an example embodiment of a data receiving device.

FIG. 2B is a block diagram illustrating an example data receiving device for communicating with the sensor according to exemplary embodiments of the disclosed subject matter.

FIGS. 2C and 2D are block diagrams depicting example embodiments of sensor control devices.

FIG. 2E is a block diagram illustrating an example analyte sensor according to exemplary embodiments of the disclosed subject matter.

FIG. 3A is a proximal perspective view depicting an example embodiment of a user preparing a tray for an assembly.

FIG. 3B is a side view depicting an example embodiment of a user preparing an applicator device for an assembly.

FIG. 3C is a proximal perspective view depicting an example embodiment of a user inserting an applicator device into a tray during an assembly.

FIG. 3D is a proximal perspective view depicting an example embodiment of a user removing an applicator device from a tray during an assembly.

FIG. 3E is a proximal perspective view depicting an example embodiment of a patient applying a sensor using an applicator device.

FIG. 3F is a proximal perspective view depicting an example embodiment of a patient with an applied sensor and a used applicator device.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 4D is a top perspective view of an exemplary applicator device in accordance with the disclosed subject matter.

FIG. 4E is a bottom perspective view of the applicator device of FIG. 4D.

FIG. 4F is an exploded view of the applicator device of FIG. 4D.

FIG. 4G is a side cutaway view of the applicator device of FIG. 4D.

FIG. 5 is a diagram illustrating example operational states of the sensor according to exemplary embodiments of the disclosed subject matter.

FIG. 6 is a diagram illustrating an example operational and data flow for over-the-air programming of a sensor according to the disclosed subject matter.

FIG. 7 is a diagram illustrating an example data flow for secure exchange of data between two devices according to the disclosed subject matter.

FIGS. 8A-8I are diagrams of a graphical user interface associated with the multimodal meal visualization system for extracting meal information, according to some embodiments.

FIGS. 9A-9C are diagrams of a graphical user interface associated with the multimodal meal visualization system for correction mechanisms associated with AI-assisted meal logging according to an embodiment.

FIGS. 10A-10D are diagrams of a graphical user interface associated with the multimodal meal visualization system for providing AI-assisted chat intent and meal selection according to an embodiment.

FIGS. 11A-11C are diagrams of a graphical user interface associated with the multimodal meal visualization system for providing AI-assisted health analysis according to an embodiment.

FIGS. 12A-12B are diagrams of a graphical user interface associated with the multimodal meal visualization system for presenting AI-assisted health insights according to an embodiment.

FIG. 13 is a block diagram of meal visualization system according to an embodiment.

FIG. 14 is a flowchart depicting steps for generating a meal visualization based on multimodal inputs according to an embodiment.

FIG. 15 is a flowchart depicting a method comprising steps for performing meal visualization analysis based on multimodal inputs according to an embodiment.

FIG. 16A is a block diagram of a system including a meal visualization model for performing meal visualization analysis according to an embodiment.

FIG. 16B is a block diagram of a system including a meal visualization model for performing meal visualization analysis according to an embodiment.

FIG. 17 is a flowchart depicting a method comprising steps for generating a meal visualization with guardrail checks according to an embodiment.

In the drawings, like reference numbers generally indicate identical or similar elements. Additionally, generally, the left-most digit(s) of a reference number identifies the drawing in which the reference number first appears.

### DETAILED DESCRIPTION

Reference will now be made in detail to systems and methods illustrated in the accompanying drawings. When a particular feature, structure, or characteristic is described in connection with an example, it is submitted that it is within the knowledge of one skilled in the art to affect such a feature, structure, or characteristic in connection with other examples whether or not explicitly described.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As discussed, the subject matter of this disclosure generally relates to systems, devices, and methods for processing multimodal inputs to extract meal information for improved efficiency and accuracy in meal logging and glucose projection, and/or for generating dynamically annotated and interactive visual representations of the multimodal inputs. Such systems are referred to herein as a “multimodal meal visualization system”, or just “meal visualization system”. The related devices and methods (including computer-implemented methods) may also be referred to as being “multimodal” or for “meal visualization.”

A multimodal meal visualization system may be used alone. Generally, however, multimodal meal visualization capabilities are used in conjunction with analyte monitoring capabilities. This is because analyte monitoring provides useful analyte data (e.g. continuous glucose data), as further discussed herein. Accordingly, in some embodiments, the multimodal meal visualization system comprises an analyte monitoring system. In other embodiments, there is provided an analyte monitoring system comprising a multimodal meal visualization system. In further embodiments, a system is provided that comprises a multimodal meal visualization system and an analyte monitoring system. Devices and methods (including computer-implemented methods) for analyte monitoring and multimodal meal visualization may similarly be combined.

### Analyte Monitoring

Various aspects of the analyte monitoring systems, devices, and methods will now be described in further detail.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor subsystem and a sharp subsystem contained therein. The user can couple the sensor subsystem to the electronics housing, and can couple the sharp to the applicator with an assembly process that involves the insertion of the applicator into the container in a specified manner. In other embodiments, the applicator, sensor control device, sensor subsystem, and sharp subsystem can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system. As used herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc. In some examples, a single analyte sensor may measure data for two or more analytes.

As mentioned, a number of embodiments of systems, devices, and methods are described herein that provide for the improved assembly and use of dermal sensor insertion devices for use with in vivo analyte monitoring systems. In particular, several embodiments of the present disclosure are designed to improve the method of sensor insertion with respect to in vivo analyte monitoring systems and, in particular, to prevent the premature retraction of an insertion sharp during a sensor insertion process. Some embodiments, for example, include a dermal sensor insertion mechanism with an increased firing velocity and a delayed sharp retraction. In other embodiments, the sharp retraction mechanism can be motion-actuated such that the sharp is not retracted until the user pulls the applicator away from the skin. Consequently, these embodiments can reduce the likelihood of prematurely withdrawing an insertion sharp during a sensor insertion process; decrease the likelihood of improper sensor insertion; and decrease the likelihood of damaging a sensor during the sensor insertion process, to name a few advantages. Several embodiments of the present disclosure also provide for improved insertion sharp subsystems to account for the small scale of dermal sensors and the relatively shallow insertion path present in a subject's dermal layer. In addition, several embodiments of the present disclosure are designed to prevent undesirable axial and/or rotational movement of applicator components during sensor insertion. Accordingly, these embodiments can reduce the likelihood of instability of a positioned dermal sensor, irritation at the insertion site, damage to surrounding tissue, and breakage of capillary blood vessels resulting in fouling of the dermal fluid with blood, to name a few advantages. In addition, to mitigate inaccurate sensor readings which can be caused by trauma at the insertion site, several embodiments of the present disclosure can reduce the end-depth penetration of the needle relative to the sensor tip during insertion.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. CGM systems, for example, can transmit data from a sensor control device to a data receiving device continuously without prompting, e.g., automatically according to a schedule via Bluetooth or Bluetooth Low Energy (“BLE” or “BTLE”). “Flash Analyte Monitoring” systems (or “Flash Glucose Monitoring” systems or simply “Flash” systems), as another example, can transfer data from a sensor control device in response to a scan or request for data by a data receiving device, such as with a Near Field Communication (“NFC”) or Radio Frequency Identification (“RFID”) protocol. In vivo analyte monitoring systems can also operate without the need for finger stick calibration.

In vivo analyte monitoring systems can be differentiated from in vitro systems that contact a biological sample outside of the body (or ex vivo) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

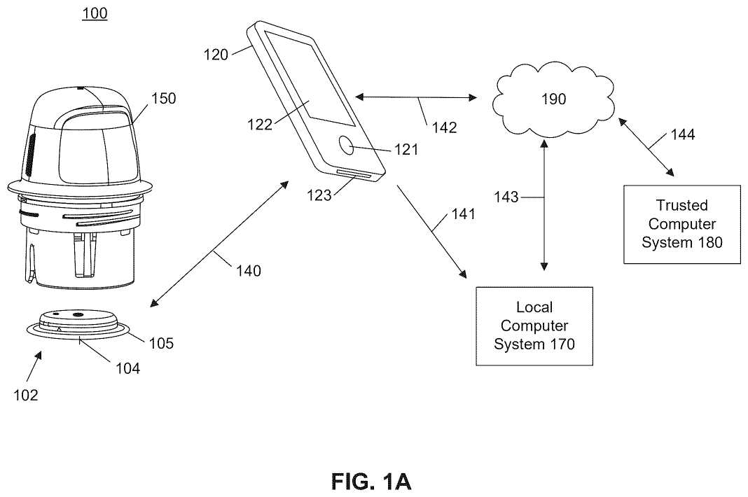

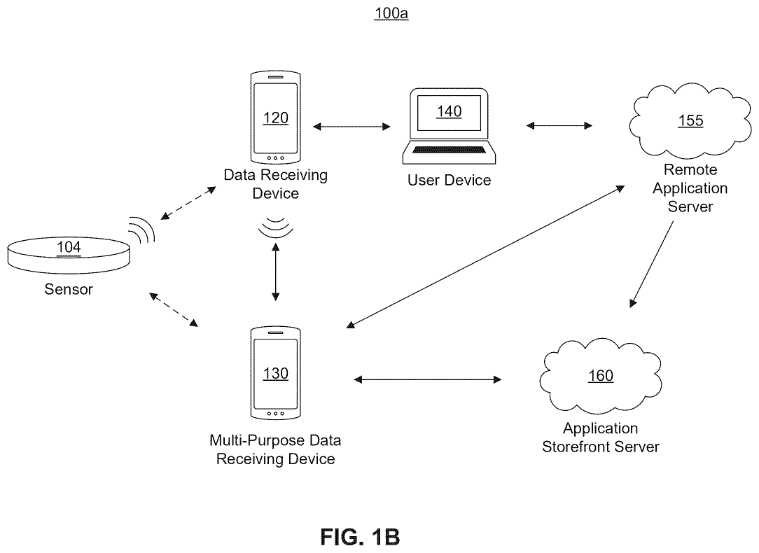

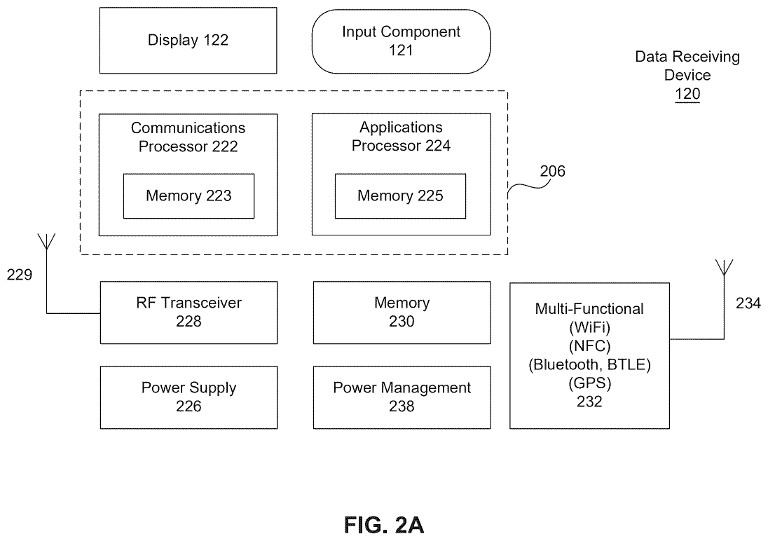

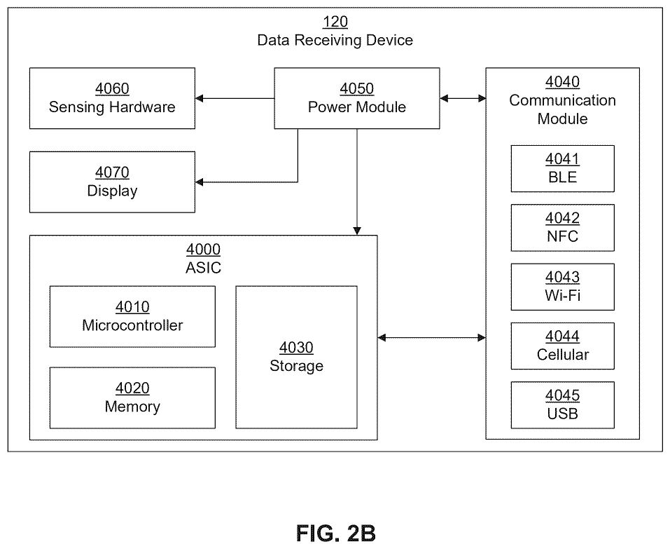

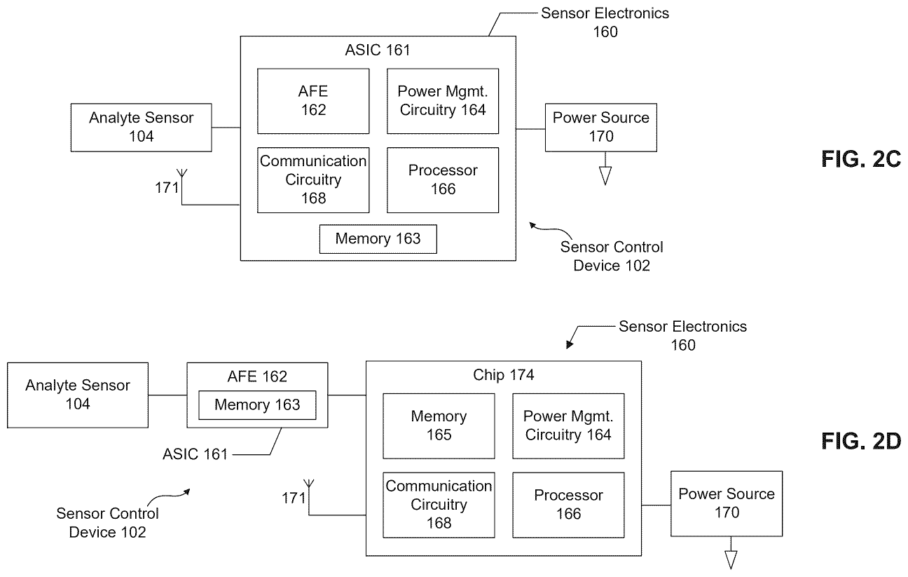

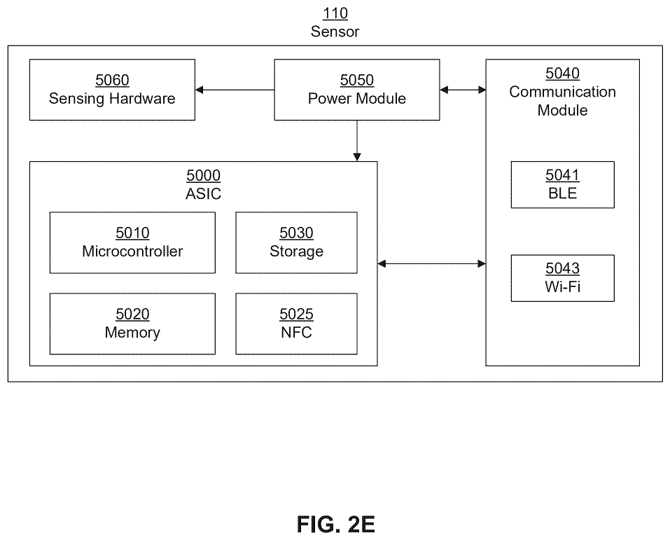

FIG. 1A is a conceptual diagram depicting an example embodiment of an analyte monitoring system 100 that includes applicator device 150, sensor control device 102, and data receiving device 120. Here, applicator device 150 can be used to deliver sensor control device 102 to a monitoring location on a user's skin where in vivo analyte sensor 104 is maintained in position for a period of time by adhesive patch 105. Sensor control device 102 is further described in FIGS. 2B and 2C, and can communicate with data receiving device 120 via a communication path 141 using a wired or wireless technique. Example wireless protocols include Bluetooth, Bluetooth Low Energy (“BLE” or “BTLE”), Bluetooth SMART, NFC, and others. Users can monitor applications installed in memory on data receiving device 120 using display 122 and input component 121 and the device battery can be recharged using power port 123. More detail about data receiving device 120 is set forth with respect to FIG. 2A below. Data receiving device 120 can communicate with local computer system 175 via a communication path 141 using a wired or wireless technique. Local computer system 175 can include one or more of a laptop, desktop, tablet, phablet, smartphone, set-top box, video game console, or other computing device and wireless communication can include any of a number of applicable wireless networking protocols including Bluetooth, BTLE, Wi-Fi or others. Local computer system 175 can communicate via communications path 143 with a network 190 similar to how data receiving device 120 can communicate via a communications path 142 with network 190, by wired or wireless technique as described previously. Network 190 can be any of a number of networks, such as private networks and public networks, local area or wide area networks, a cloud network, and so forth. A trusted computer system 180, which may be a cloud-based system, can include a server and can provide authentication services and secured data storage and can communicate via communications path 144 with network 190 by wired or wireless technique. In some embodiments, data receiving device 120 may be implemented as a mobile device, such as a smartphone, tablet, or watch device.

FIG. 1B illustrates an operating environment of analyte monitoring system 100a capable of embodying the techniques described herein. Analyte monitoring system 100a may include a system of components designed to provide monitoring of parameters, such as analyte levels, of a human or animal body or can provide for other operations based on the configurations of the various components. As embodied herein, the system may include analyte sensor 104, or simply “sensor” worn by the user or attached to the body for which information is being collected. As embodied herein, analyte sensor 104 can be a sealed, disposable device with a predetermined active use lifetime (e.g., 1 day, 14 days, 15 days, 30 days, etc.). Analyte sensor 104 may be applied to the skin of the user body and remain adhered over the duration of the sensor lifetime or can be designed to be selectively removed and remain functional when reapplied. Analyte monitoring system 100a can further include data receiving device 120 or multi-purpose data receiving device 130 configured as described herein to facilitate retrieval and delivery of data, including analyte data, from analyte sensor 104.

As embodied herein, analyte monitoring system 100a can include a software or firmware library or application provided, for example via remote application server 155 or application storefront server 160, to a third-party and incorporated into multi-purpose data receiving device 130 such as a mobile phone, tablet, personal computing device, or other similar computing device capable of communicating with analyte sensor 104 over a communication link. Multi-purpose hardware can further include embedded devices, including, but not limited to insulin pumps or insulin pens, having an embedded library configured to communicate with analyte sensor 104. Although the illustrated embodiments of analyte monitoring system 100a include only one of each of the illustrated devices, this disclosure contemplates analyte monitoring system 100a incorporating multiples of each component interacting throughout the system. For example and without limitation, as embodied herein, data receiving device 120 and/or multi-purpose data receiving device 130 can include multiples of each. As embodied herein, multi-purpose data receiving device 130 can communicate directly with analyte sensor 104 as described herein. Additionally or alternatively, multi-purpose data receiving device 130 can communicate with multi-purpose data receiving device 130 to provide analyte data, or visualization or analysis of the data, for secondary display to the user or other authorized parties.

FIG. 1B further illustrates an example operating environment for providing over-the-air (“OTA”) updates for use with the techniques described herein. An operator of analyte monitoring system 100 can bundle updates for data receiving device 120 or analyte sensor 104 into updates for an application executing on multi-purpose data receiving device 130. Using available communication channels between data receiving device 120, multi-purpose data receiving device 130, and analyte sensor 104, multi-purpose data receiving device 130 can receive regular updates for data receiving device 120 or analyte sensor 104 and initiate installation of the updates on data receiving device 120 or analyte sensor 104. Multi-purpose data receiving device 130 acts as an installation or update platform for data receiving device 120 or analyte sensor 104 because the application that enables the multi-purpose data receiving device 130 to communicate with analyte sensor 104, data receiving device 120 and/or remote application server 155 can update software or firmware on data receiving device 120 or analyte sensor 104 without wide-area networking capabilities.

As embodied herein, remote application server 155 operated by the manufacturer of analyte sensor 104 and/or the operator of analyte monitoring system 100 can provide software and firmware updates to the devices of analyte monitoring system 100. In particular embodiments, remote application server 155 can provides the updated software and firmware to user device 140 or directly to a multi-purpose data receiving device. As embodied herein, remote application server 155 can also provide application software updates to application storefront server 160 using interfaces provided by the application storefront. Multi-purpose data receiving device 130 can contact the application storefront server 160 periodically to download and install the updates.

After multi-purpose data receiving device 130 downloads an application update including a firmware or software update for data receiving device 120 or analyte sensor 104, data receiving device 120 or analyte sensor 104 and multi-purpose data receiving device 130 establish a connection. Multi-purpose data receiving device 130 determines that a firmware or software update is available for data receiving device 120 or analyte sensor 104. Multi-purpose data receiving device 130 can prepare the software or firmware update for delivery to data receiving device 120 or analyte sensor 104. As an example, multi-purpose data receiving device 130 can compress or segment the data associated with the software or firmware update, can encrypt or decrypt the firmware or software update, or can perform an integrity check of the firmware or software update. Multi-purpose data receiving device 130 sends the data for the firmware or software update to data receiving device 120 or analyte sensor 104. Multi-purpose data receiving device 130 can also send a command to data receiving device 120 or analyte sensor 104 to initiate the update. Additionally or alternatively, multi-purpose data receiving device 130 can provide a notification to the user of multi-purpose data receiving device 130 and include instructions for facilitating the update, such as instructions to keep data receiving device 120 and multi-purpose data receiving device 130 connected to a power source and in close proximity until the update is complete.

Data receiving device 120 or analyte sensor 104 receives the data for the update and the command to initiate the update from multi-purpose data receiving device 130. Data receiving device 120 can then install the firmware or software update. To install the update, data receiving device 120 or analyte sensor 104 can place or restart itself in a so-called “safe” mode with limited operational capabilities. Once the update is completed, data receiving device 120 or analyte sensor 104 re-enters or resets into a standard operational mode. Data receiving device 120 or analyte sensor 104 can perform one or more self-tests to determine that the firmware or software update was installed successfully. Multi-purpose data receiving device 130 can receive the notification of the successful update. Multi-purpose data receiving device 130 can then report a confirmation of the successful update to remote application server 155.

In particular embodiments, storage memory 5030 of analyte sensor 104 includes OTP memory. The term OTP memory can refer to memory that includes access restrictions and security to facilitate writing to particular addresses or segments in the memory a predetermined number of times. Memory 5030 can be prearranged into multiple pre-allocated memory blocks or containers. The containers are pre-allocated into a fixed size. If storage memory 5030 is OTP memory, the containers can be considered to be in a non-programmable state. Additional containers which have not yet been written to can be placed into a programmable or writable state. Containerizing storage memory 5030 in this fashion can improve the transportability of code and data to be written to storage memory 5030. Updating the software of a device (e.g., the sensor device described herein) stored in an OTP memory can be performed by superseding only the code in a particular previously-written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

### Exemplary Data Receiving Device

FIG. 2A is a block diagram depicting an example embodiment of a data receiving device 120 configured as a smartphone. Here, data receiving device 120 can include a display 122, input component 121, and processing core 206 including communications processor 222 coupled with memory 223 and applications processor 224 coupled with memory 225. Also included can be separate memory 230, RF transceiver 228 with antenna 229, and power supply 226 with power management subsystem 238. Further included can be a multi-functional transceiver 232 which can communicate over Wi-Fi, NFC, Bluetooth, BTLE, and GPS with antenna 234. As understood by one of skill in the art, these components are electrically and communicatively coupled in a manner to make a functional device.

Memory 230 may be configured to store the medical application (e.g., CGM application) and a meal visualization model. Optionally, the meal visualization model may be a machine learning model that can be trained remotely from data receiving device, such as at remote application server 155 or any other server associated with the medical application. The meal visualization model may be configured to generate proposed user interface modifications to the evolving medical user interface (EMUI) of the medical application installed on data receiving device 120. The meal visualization model may be implemented as subsystem of the medical application and both can be configured to receive and process medical data including real-time medical data from a medical or analyte sensor (e.g., CGM sensor) and historical medical data (e.g., stored on the data receiving device 120 or remotely, such as at a backend database or server, where electronic health records for the user may be retrieved). The meal visualization model and/or the medical application may also be granted access privileges to user interactions with data receiving device 120 that include, for example, other applications installed on the data receiving device 120, duration of user interaction with the data receiving device 120 and/or the other installed applications, and date and time of user interaction with the data receiving device 120 and/or the other installed applications. The meal visualization model and/or the medical application may also be configured to connect with databases remote from data receiving device 120 that may include historical medical data, such as medical history from an HCP, as well as cohort data that represents anonymized medical data collected from the medical sensors of other users. A cohort may be established for the current user based on one or more similar characteristics to the current user, such as, user age, user medical condition, user medical history, user location, and user preference, just to name a few examples.

### Exemplary Data Receiving Device Architecture

For purpose of illustration and not limitation, reference is made to the exemplary embodiment of data receiving device 120 for use with the disclosed subject matter as shown in FIG. 2B. Data receiving device 120 and the related multi-purpose data receiving device 130 include components germane to the discussion of analyte sensor 104 and its operations and additional components can be included. Data receiving device 120 may also be implemented with components for generating different interface elements of the EMUI that is displayed to the user. For example, data receiving device 120 may receive and store a meal visualization model trained to receive any combination of user analyte data, user medical history, and other user activity data (e.g., device interaction, tracked physical activity, tracked meals) and generate the interface elements for the EMUI. In particular embodiments, data receiving device 120 and multi-purpose data receiving device 130 can be or include components provided by a third party and are not necessarily restricted to include devices made by the same manufacturer as analyte sensor 104.

As illustrated in FIG. 2B, data receiving device 120 includes ASIC 4000 having microcontroller 4010, memory 4020, and storage 4030 and communicatively coupled with a communication subsystem 4040. Power for the components of data receiving device 120 can be delivered by power subsystem 4050, which as embodied herein can include a rechargeable battery. Data receiving device 120 can further include display 4070 for facilitating review of analyte data received from analyte sensor 104 or other device (e.g., user device 140 or remote application server 155). Data receiving device 120 can include separate user interface components (e.g., physical keys, light sensors, microphones, etc.).

Memory 4020 may be configured to store a medical application (e.g., CGM application) that is configured to communicate with and receive data from the analyte sensor of the user. The medical application may further be implemented with a meal visualization model, similar to the configuration discussed with regard to FIG. 2A. The medical application may be one of: a monitoring application of a continuous glucose monitor (CGM) (or any other analyte), a standalone application, a standalone application which is not a monitoring application of a continuous glucose monitor (CGM) (or any other analyte), and an insulin delivery application.

Communication subsystem 4040 can include BLE subsystem 4041 and NFC subsystem 4042. Data receiving device 120 can be configured to wirelessly couple with analyte sensor 104 and transmit commands to and receive data from analyte sensor 104. As embodied herein, data receiving device 120 can be configured to operate, with respect to analyte sensor 104 as described herein, as an NFC scanner and a BLE end point via specific subsystems (e.g., BLE subsystem 4042 or NFC subsystem 4043) of communication subsystem 4040. For example, data receiving device 120 can issue commands (e.g., activation commands for a data broadcast mode of the sensor; pairing commands to identify data receiving device 120) to analyte sensor 104 using a first subsystem of the communication subsystem 4040 and receive data from and transmit data to analyte sensor 104 using a second subsystem of the communication subsystem 4040. Data receiving device 120 can be configured for communication with user device 140 via a Universal Serial Bus (“USB”) subsystem 4045 of the communication subsystem 4040.

As another example, communication subsystem 4040 can include, for example, cellular radio subsystem 4044. The cellular radio subsystem 4044 can include one or more radio transceivers for communicating using broadband cellular networks, including, but not limited to third generation (“3G”), fourth generation (“4G”), and fifth generation (“5G”) networks. Additionally, communication subsystem 4040 of data receiving device 120 can include Wi-Fi radio subsystem 4043 for communication using a wireless local area network according to one or more of the IEEE 802.11 standards (e.g., 802.11a, 802.11b, 802.11g, 802.11n (aka Wi-Fi 4), 802.11ac (aka Wi-Fi 5), 802.11ax (aka Wi-Fi 6)). Using cellular radio subsystem 4044 or Wi-Fi radio subsystem 4043, data receiving device 120 can communicate with remote application server 155 to receive analyte data or provide updates or input received from a user (e.g., through one or more user interfaces). Although not illustrated, communication subsystem 5040 of analyte sensor 104 can similarly include a cellular radio subsystem or Wi-Fi radio subsystem.

As embodied herein, on-board storage 4030 of data receiving device 120 can store analyte data received from analyte sensor 104. Further, data receiving device 120, multi-purpose data receiving device 130, or user device 140 can be configured to communicate with remote application server 155 via a wide area network. As embodied herein, analyte sensor 104 can provide data to data receiving device 120 or multi-purpose data receiving device 130. Data receiving device 120 can transmit the data to user device 140. User device 140 (or multi-purpose data receiving device 130) can in turn transmit that data to remote application server 155 for processing and analysis.

As embodied herein, data receiving device 120 can further include sensing hardware 4060 similar to, or expanded from, sensing hardware 5060 of analyte sensor 104. In particular embodiments, data receiving device 120 can be configured to operate in coordination with analyte sensor 104 and based on analyte data received from the analyte sensor 104. As an example, where analyte sensor 104 is a glucose sensor, data receiving device 120 can be or include an insulin pump, insulin injection pen, or a smart insulin pen cap. In coordination, multi-purpose data receiving device 130 can adjust an insulin dosage for a user based on glucose values received from the analyte sensor.

### Exemplary Sensor Control Devices

FIGS. 2C and 2D are block diagrams depicting example embodiments of sensor control device 102 having in vivo analyte sensor 104 and sensor electronics 169 (including analyte monitoring circuitry) that can have the majority of the processing capability for rendering end-result data suitable for display to the user. In FIG. 2C, a single semiconductor chip 161 is depicted that can be a custom application specific integrated circuit (“ASIC”). Shown within ASIC 161 are certain high-level functional units, including an analog front end (“AFE”) 162, power management circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

Memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to data receiving device 120 (not shown), for example, where minimal further processing is needed by the resident software application to display the data.

FIG. 2D is similar to FIG. 2C but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

For purpose of illustration and not limitation, reference is made to the exemplary embodiment of analyte sensor 104 for use with the disclosed subject matter as shown in FIG. 2E. FIG. 2E illustrates a block diagram of an example analyte sensor 104 according to exemplary embodiments compatible with the security architecture and communication schemes described herein.

As embodied herein, analyte sensor 104 can include ASIC 5000 communicatively coupled with a communication subsystem 5040. ASIC 5000 can include a microcontroller core 5010, on-board memory 5020, and storage memory 5030. Storage memory 5030 can store data used in an authentication and encryption security architecture. Storage memory 5030 can store programming instructions for analyte sensor 104. As embodied herein, certain communication chipsets can be embedded in the ASIC 5000 (e.g., an NFC transceiver 5025). ASIC 5000 can receive power from a power subsystem 5050, such as an on-board battery or from an NFC pulse. Storage memory 5030 of the ASIC 5000 can be programmed to include information such as an identifier for analyte sensor 104 for identification and tracking purposes. Storage memory 5030 can also be programmed with configuration or calibration parameters for use by analyte sensor 104 and its various components. Storage memory 5030 can include rewritable or one-time programming (“OTP”) memory. The storage memory 5030 can be updated using techniques described herein to extend the usefulness of analyte sensor 104.

As embodied herein, communication subsystem 5040 of sensor 104 can be or include one or more subsystems to support analyte sensor 104 communicating with other devices of the analyte monitoring system 100. As an example only and not by way of limitation, example communication subsystems 5040 can include BLE subsystem 5041 As used throughout this disclosure, BLE refers to a short-range communication protocol optimized to make pairing of Bluetooth devices simple for end users. Communication subsystem 5040 can transmit and receive data and commands via interaction with similarly-capable communication subsystems of data receiving device 120 or user device 140. Communication subsystem 5040 can include additional or alternative chipsets for use with similar short-range communication schemes, such as a personal area network according to IEEE 802.15 protocols, IEEE 802.11 protocols, infrared communications according to the Infrared Data Association standards (IrDA), etc.

To perform its functionalities, sensor 104 can further include suitable sensing hardware 5060 appropriate to its function. As embodied herein, sensing hardware 5060 can include an analyte sensor transcutaneously or subcutaneously positioned in contact with a bodily fluid of a subject. The analyte sensor can generate sensor data containing values corresponding to levels of one or more analytes within the bodily fluid.

### Exemplary Assembly Processes for Sensor Control Devices

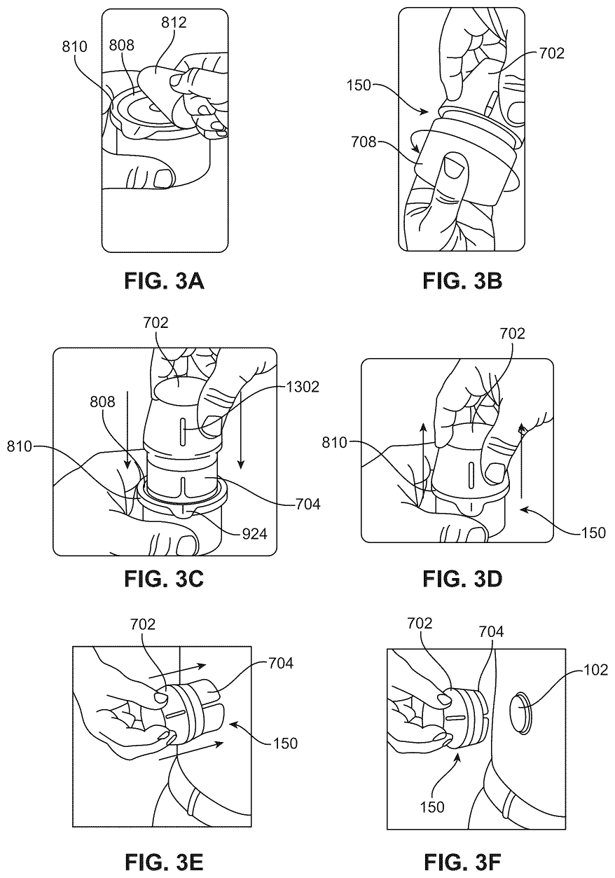

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of user preparing tray 810, configured here as a tray (although other packages/containers can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator device 150 for assembly. Applicator device 150 can be provided in a sterile package sealed by a cap 708. Preparation of applicator device 150 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting applicator device 150 into tray 810 during an assembly. Initially, the user can insert sheath 704 into platform 808 inside tray 810 after aligning a housing orienting feature (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator device 150 from tray 810 will result in the same state prior to initial insertion of applicator device 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the applicator device 150 and tray 810 are further described below.

FIG. 3D is a proximal perspective view depicting an example embodiment of a user removing an applicator device 150 from tray 810 during an assembly. A user can remove applicator device 150 from tray 810 by proximally advancing housing 702 with respect to tray 810 or other motions having the same end effect of uncoupling applicator device 150 and tray 810. The applicator device 150 is removed with sensor control device 102 (not shown) fully assembled (sharp, sensor, electronics) therein and positioned for delivery.

FIG. 3E is a proximal perspective view depicting an example embodiment of a patient applying sensor control device 102 using applicator device 150 to a target area of skin, for instance, on an abdomen or other appropriate location. Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an applied position. The user can then remove applicator device 150 from the application site.

Analyte monitoring system 100, described with respect to FIGS. 3A-3F and elsewhere herein, can provide a reduced or eliminated chance of accidental breakage, permanent deformation, or incorrect assembly of applicator components compared to prior art systems. Since housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Exemplary Sensor Applicator Devices

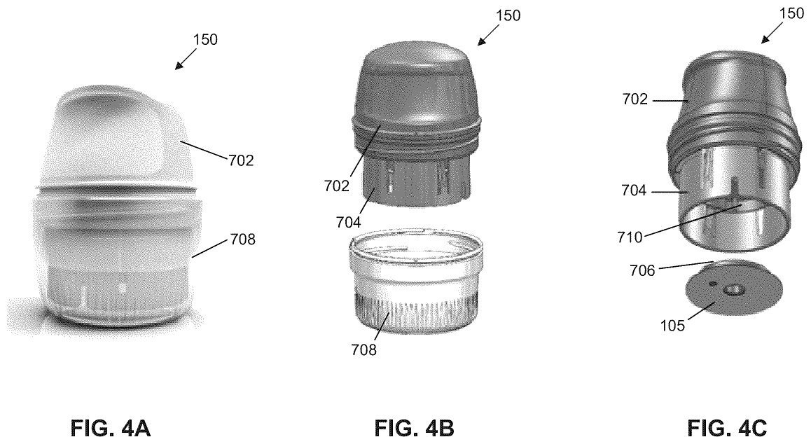

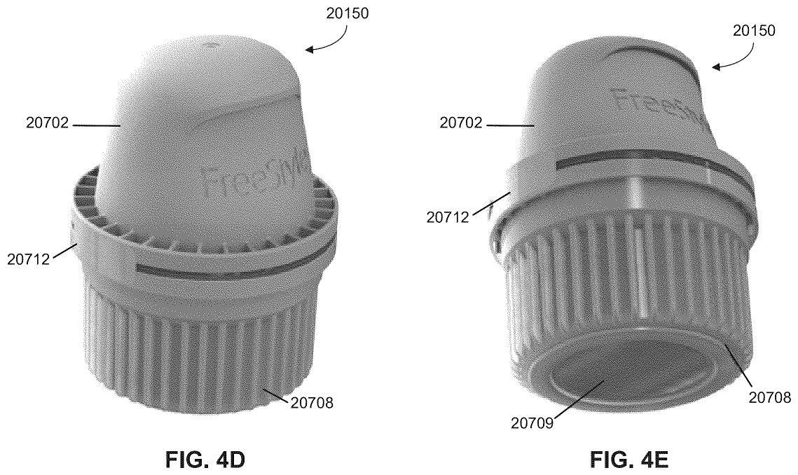

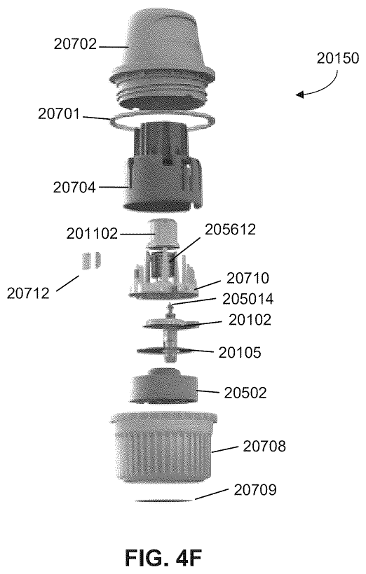

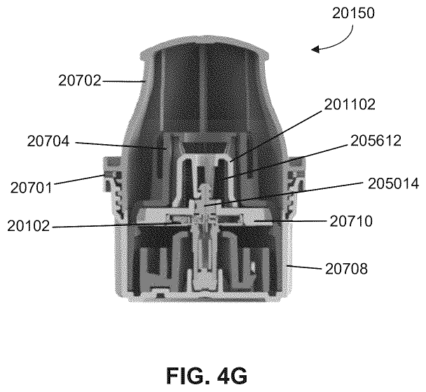

FIG. 4A is a side view depicting an example embodiment of an applicator device 150 coupled with screw cap 708. This is an example of how applicator device 150 is shipped to and received by a user, prior to assembly by the user with a sensor. FIG. 4B is a side perspective view depicting applicator device 150 and cap 708 after being decoupled. FIG. 4C is a perspective view depicting an example embodiment of a distal end of applicator device 150 with electronics housing 706 and adhesive patch 105 removed from the position they would have retained within sensor carrier 710 of sheath 704, when cap 708 is in place.

Referring to FIG. 4D-G for purpose of illustration and not limitation, applicator device 20150 can be provided to a user as a single integrated assembly. FIGS. 4D and 4E provide perspective top and bottom views, respectively, of the applicator device 20150, FIG. 4F provides an exploded view of applicator device 20150 and FIG. 4G provides a side cut-away view. The perspective views illustrate how applicator device 20150 is shipped to and received by a user. The exploded and cut-away views illustrate the components of applicator device 20150. The applicator device 20150 can include a housing 20702, gasket 20701, sheath 20704, sharp carrier 201102, spring 205612, sensor carrier 20710 (also referred to as a “puck carrier”), sharp hub 205014, sensor control device (also referred to as a “puck”) 20102, adhesive patch 20105, desiccant 20502, cap 20708, serial label 20709, and tamper evidence feature 20712. As received by a user, only housing 20702, cap 20708, tamper evidence feature 20712, and label 20709 are visible. The tamper evidence feature 20712 can be, for example, a sticker coupled to each of housing 20702 and cap 20708, and tamper evidence feature 20712 can be damaged, for example, irreparably, by uncoupling housing 20702 and cap 20708, thereby indicating to a user that housing 20702 and cap 20708 have been previously uncoupled. These features are described in greater detail below.

### Exemplary Sensor States and Activation

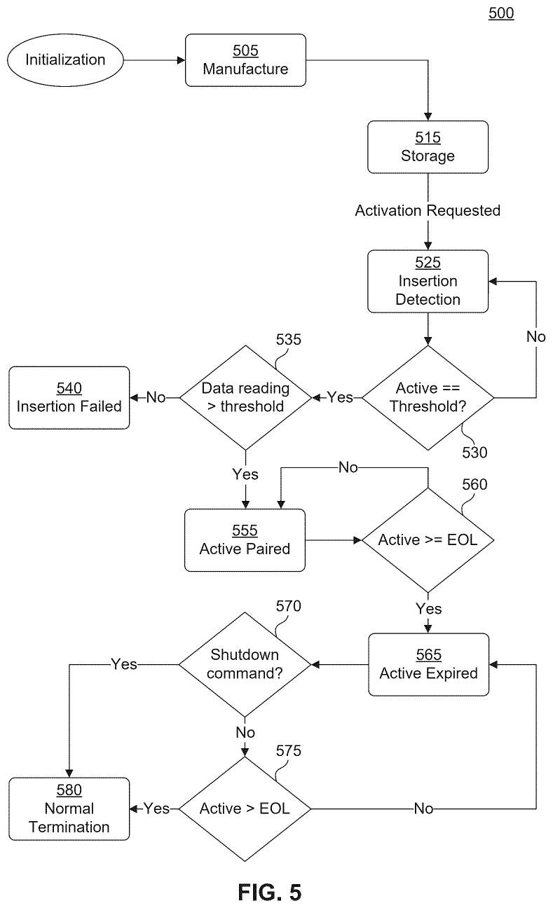

For purpose of illustration and not limitation, reference is made to the exemplary embodiment of a high-level depiction of a state machine representation 500 of the actions that can be taken by analyte sensor 104 as shown in FIG. 5. After initialization, the sensor enters state 505, which relates to the manufacture of analyte sensor 104. In the manufacture state 505, analyte sensor 104 can be configured for operation, for example, the storage memory 5030 can be written. At various times while in state 505, analyte sensor 104 checks for a received command to go to storage state 515. Upon entry to storage state 515, the sensor performs a software integrity check. While in storage state 515, the sensor can also receive an activation request command before advancing to insertion detection state 525.

Upon entry to state 525, analyte sensor 104 can store information relating to devices authenticated to communicate with the sensor as set during activation or initialize algorithms related to conducting and interpreting measurements from sensing hardware 5060. Analyte sensor 104 can also initialize a lifecycle timer, responsible for maintaining an active count of the time of operation of analyte sensor 104 and begin communication with authenticated devices to transmit recorded data. While in insertion detection state 525, the sensor can enter state 530, where analyte sensor 104 checks whether the time of operation is equal to a predetermined threshold. This time of operation threshold can correspond to a timeout function for determining whether an insertion has been successful. If the time of operation has reached the threshold, analyte sensor 104 advances to state 535, in which analyte sensor 104 checks whether the average data reading is greater than a threshold amount corresponding to an expected data reading volume for triggering detection of a successful insertion. If the data reading volume is lower than the threshold while in state 535, the sensor advances to state 540, corresponding to a failed insertion. If the data reading volume satisfies the threshold, the sensor advances to active paired state 555.

Active paired state 555 of analyte sensor 104 reflects the state while analyte sensor 104 is operating as normal by recording measurements, processing the measurements, and reporting them as appropriate. While in active paired state 555, analyte sensor 104 sends measurement results or attempts to establish a connection with data receiving device 120. Analyte sensor 104 also increments the time of operation. Once analyte sensor 104 reaches a predetermined threshold time of operation (e.g., once the time of operation reaches a predetermined threshold), analyte sensor 104 transitions to active expired state 565. Active expired state 565 of analyte sensor 104 reflects the state while analyte sensor 104 has operated for its maximum predetermined amount of time.

While in active expired state 565, analyte sensor 104 can generally perform operations relating to winding down operation and ensuring that the collected measurements have been securely transmitted to receiving devices as needed. For example, while in active expired state 565, analyte sensor 104 can transmit collected data and, if no connection is available, can increase efforts to discover authenticated devices nearby and establish and connection therewith. While in active expired state 565, analyte sensor 104 can receive a shutdown command at state 570. If no shutdown command is received, analyte sensor 104 can also, at state 575, check if the time of operation has exceeded a final operation threshold. The final operation threshold can be based on the battery life of analyte sensor 104. The normal termination state 580 corresponds to the final operations of analyte sensor 104 and ultimately shutting down analyte sensor 104.

Before a sensor is activated, ASIC 5000 resides in a low power storage mode state. The activation process can begin, for example, when an incoming RF field (e.g., NFC field) drives the voltage of the power supply to the ASIC 5000 above a reset threshold, which causes analyte sensor 104 to enter a wake-up state. While in the wake-up state, ASIC 5000 enters an activation sequence state. ASIC 5000 then wakes communication subsystem 5040. Communication subsystem 5040 is initialized, triggering a power on self-test. The power on self-test can include ASIC 5000 communicating with communication subsystem 5040 using a prescribed sequence of reading and writing data to verify the memory and one-time programmable memory are not corrupted.

When ASIC 5000 enters the measurement mode for the first time, an insertion detection sequence is performed to verify that analyte sensor 104 has been properly installed onto the patient's body before a proper measurement can take place. First, analyte sensor 104 interprets a command to activate the measurement configuration process, causing the ASIC 5000 to enter measurement command mode. Analyte sensor 104 then temporarily enters the measurement lifecycle state to run a number of consecutive measurements to test whether the insertion has been successful. Communication subsystem 5040 or ASIC 5000 evaluates the measurement results to determine insertion success. When insertion is deemed successful, analyte sensor 104 enters a measurement state, in which analyte sensor 104 begins taking regular measurements using sensing hardware 5060. If analyte sensor 104 determines that the insertion was not successful, analyte sensor 104 is triggered into an insertion failure mode, in which ASIC 5000 is commanded back to storage mode while communication subsystem 5040 disables itself.

### Exemplary Over-the-Air Updates

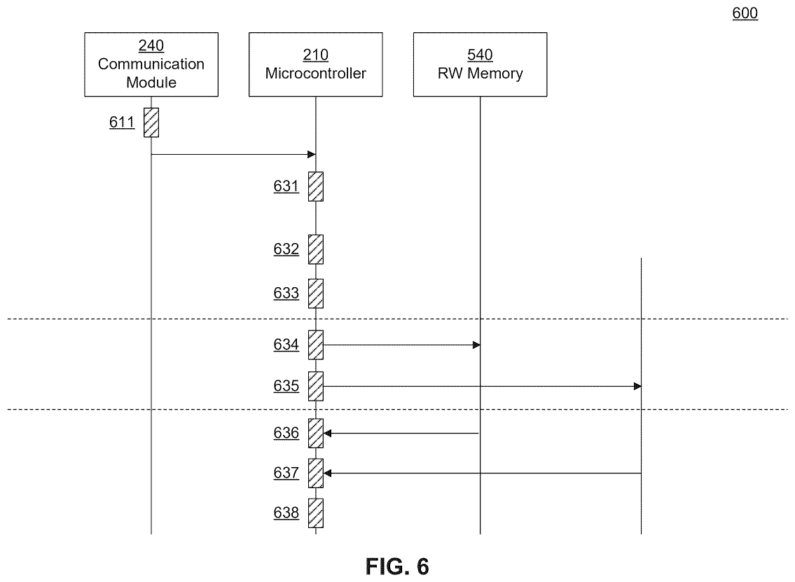

FIG. 6 is a diagram illustrating an example operational and data flow for over-the-air (“OTA”) programming of storage memory 5030 in analyte monitoring system 100 as well as use of the memory after the OTA programming in execution of processes by analyte sensor 104 according to the disclosed subject matter. In the example OTA programming 600 illustrated in FIG. 6, a request is sent from an external device (e.g., multi-purpose data receiving device 130) to initiate OTA programming (or re-programming). At 611, communication subsystem 240 of analyte sensor 104 receives an OTA programming command. Communication subsystem 240 sends the OTA programming command to the microcontroller 210 of analyte sensor 104.

At 631, after receiving the OTA programming command, microcontroller 210 validates the OTA programming command. Microcontroller 210 can determine, for example, whether the OTA programming command is signed with an appropriate digital signature token. Upon determining that the OTA programming command is valid, microcontroller 210 can set the sensor device into an OTA programming mode. At 632, microcontroller 210 can validate the OTA programming data. At 633, microcontroller 210 can reset analyte sensor 104 to re-initialize analyte sensor 104 in a programming state. Once analyte sensor 104 has transitioned into the OTA programming state, microcontroller 210 can begin to write data to the rewriteable memory (e.g., memory 5020) of the sensor device at 634 and write data to OTP memory 550 of the sensor device at 635 (e.g., storage memory 5030). The data written by the microcontroller 210 can be based on the validated OTA programming data. Microcontroller 210 can write data to cause one or more programming blocks or regions of OTP memory 550 to be marked invalid or inaccessible. The data written to the free or unused portion of OTP memory 550 can be used to replace invalidated or inaccessible programming blocks of OTP memory 550. After microcontroller 210 writes the data to the respective memories at 634 and 635, microcontroller 210 can perform one or more software integrity checks to ensure that errors were not introduced into the programming blocks during the writing process. Once microcontroller 210 is able to determine that the data has been written without errors, microcontroller 210 can resume standard operations of the sensor device.

In execution mode, at 636, microcontroller 210 can retrieve a programming manifest or profile from the rewriteable memory. The programming manifest or profile can include a listing of the valid software programming blocks and can include a guide to program execution for analyte sensor 104. By following the programming manifest or profile, microcontroller 210 can determine which memory blocks of OTP memory 550 are appropriate to execute and avoid execution of out-of-date or invalidated programming blocks or reference to out-of-date data. At 637, microcontroller 210 can selectively retrieve memory blocks from OTP memory 550. At 638, microcontroller 210 can use the retrieved memory blocks, by executing programming code stored or using variable stored in the memory.

### Exemplary Security and Other Architecture Features

As embodied herein a first layer of security for communications between analyte sensor 104 and other devices can be established based on security protocols specified by and integrated in the communication protocols used for the communication. Another layer of security can be based on communication protocols that necessitate close proximity of communicating devices. Furthermore certain packets and/or certain data included within packets can be encrypted while other packets and/or data within packets is otherwise encrypted or not encrypted. Additionally or alternatively, application layer encryption can be used with one or more block ciphers or stream ciphers to establish mutual authentication and communication encryption with other devices in the analyte monitoring system 100.

ASIC 5000 of analyte sensor 104 can be configured to dynamically generate authentication and encryption keys using data retained within storage memory 5030. Storage memory 5030 can also be pre-programmed with a set of valid authentication and encryption keys to use with particular classes of devices. ASIC 5000 can be further configured to perform authentication procedures with other devices using received data and apply the generated key to sensitive data prior to transmitting the sensitive data. The generated key can be unique to analyte sensor 104, unique to a pair of devices, unique to a communication session between analyte sensor 104 and other device, unique to a message sent during a communication session, or unique to a block of data contained within a message.

Both analyte sensor 104 and data receiving device 120 can ensure the authorization of the other party in a communication session to, for example, issue a command or receive data. In particular embodiments, identity authentication can be performed through two features. First, the party asserting its identity provides a validated certificate signed by the manufacturer of the device or the operator of analyte monitoring system 100. Second, authentication can be enforced through the use of public keys and private keys, and shared secrets derived therefrom, established by the devices of analyte monitoring system 100 or established by the operator of the analyte monitoring system 100. To confirm the identity of the other party, the party can provide proof that the party has control of its private key.

The manufacturer of analyte sensor 104, data receiving device 120, or provider of the application for multi-purpose data receiving device 130 can provide information and programming necessary for the devices to securely communicate through secured programming and updates. For example, the manufacturer can provide information that can be used to generate encryption keys for each device, including secured root keys for analyte sensor 104 and optionally for data receiving device 120 that can be used in combination with device-specific information and operational data (e.g., entropy-based random values) to generate encryption values unique to the device, session, or data transmission as need.

Analyte data associated with a user is sensitive data at least in part because this information can be used for a variety of purposes, including for health monitoring and medication dosing decisions. In addition to user data, analyte monitoring system 100 can enforce security hardening against efforts by outside parties to reverse-engineering. Communication connections can be encrypted using a device-unique or session-unique encryption key. Encrypted communications or unencrypted communications between any two devices can be verified with transmission integrity checks built into the communications. Analyte sensor 104 operations can be protected from tampering by restricting access to read and write functions to the memory 5020 via a communication interface. The sensor can be configured to grant access only to known or “trusted” devices, provided in a “whitelist” or only to devices that can provide a predetermined code associated with the manufacturer or an otherwise authenticated user. A whitelist can represent an exclusive range, meaning that no connection identifiers besides those included in the whitelist will be used, or a preferred range, in which the whitelist is searched first, but other devices can still be used. Analyte sensor 104 can further deny and shut down connection requests if the requestor cannot complete a login procedure over a communication interface within a predetermined period of time (e.g., within four seconds). These characteristics safeguard against specific denial of service attacks, and in particular against denial of service attacks on a BLE interface.

As embodied herein, analyte monitoring system 100 can employ periodic key rotation to further reduce the likelihood of key compromise and exploitation. A key rotation strategy employed by analyte monitoring system 100 can be designed to support backward compatibility of field-deployed or distributed devices. As an example, analyte monitoring system 100 can employ keys for downstream devices (e.g., devices that are in the field or cannot be feasibly provided updates) that are designed to be compatible with multiple generations of keys used by upstream devices.

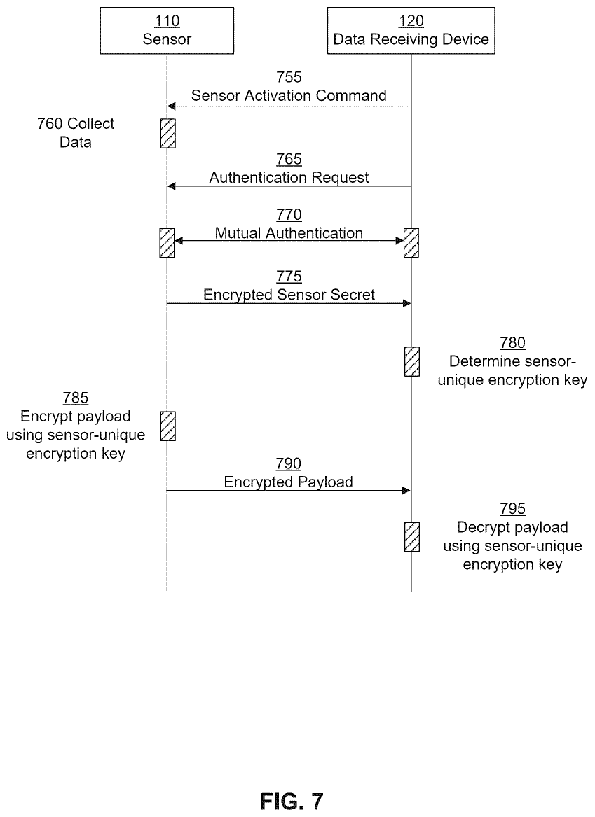

For purpose of illustration and not limitation, reference is made to the exemplary embodiment of a message sequence diagram for use with the disclosed subject matter as shown in FIG. 7 and demonstrating an example exchange of data between a pair of devices, particularly analyte sensor 104 and data receiving device 120. Data receiving device 120 can, as embodied herein, be data receiving device 120 or multi-purpose data receiving device 130. At step 755, data receiving device 120 can transmit a sensor activation command to analyte sensor 104, for example via a short-range communication protocol. Analyte sensor 104 can, prior to step 755 be in a primarily dormant state, preserving its battery until full activation is needed. After activation during step 760, analyte sensor 104 can collect data or perform other operations as appropriate to the sensing hardware 5060 of analyte sensor 104. At step 765, data receiving device 120 can an initiate authentication request command. In response to the authentication request command, both analyte sensor 104 and data receiving device 120 can engage in a mutual authentication process in step 770. Step 770 can involve the transfer of data, including challenge parameters that allow analyte sensor 104 and data receiving device 120 to ensure that the other device is sufficiently capable of adhering to an agreed-upon security system described herein. Mutual authentication can be based on mechanisms for authentication of two or more entities to each other with or without on-line trusted third parties to verify establishment of a secret key via challenge-response. Mutual authentication can be performed using two-, three-, four-, or five-pass authentication, or similar versions thereof.

Following a successful step 770, at step 775 analyte sensor 104 can provide data receiving device 120 with a sensor secret. The sensor secret can contain sensor-unique values and be derived from random values generated during manufacture. The sensor secret can be encrypted prior to or during transmission to prevent third-parties from accessing the secret. The sensor secret can be encrypted via one or more of the keys generated by or in response to the mutual authentication process. At step 780, data receiving device 120 can derive a sensor-unique encryption key from the sensor secret. The sensor-unique encryption key can further be session-unique. As such, the sensor-unique encryption key can be determined by each device without being transmitted between analyte sensor 104 or data receiving device 120. At step 785, analyte sensor 104 can encrypt data to be included in payload. At step 790, analyte sensor 104 can transmit the encrypted payload 740 to data receiving device 120 using the communication link established between the appropriate communication models of analyte sensor 104 and data receiving device 120. At step 795, data receiving device 120 can decrypt the payload using the sensor-unique encryption key derived during step 780. Following step 795, analyte sensor 104 can deliver additional (including newly collected) data and data receiving device 120 can process the received data appropriately.

As discussed herein, analyte sensor 104 can be a device with restricted processing power, battery supply, and storage. The encryption techniques used by analyte sensor 104 (e.g., the cipher algorithm or the choice of implementation of the algorithm) can be selected based at least in part on these restrictions. Data receiving device 120 can be a more powerful device with fewer restrictions of this nature. Therefore, data receiving device 120 can employ more sophisticated, computationally intense encryption techniques, such as cipher algorithms and implementations.

### Multimodal Meal Visualization

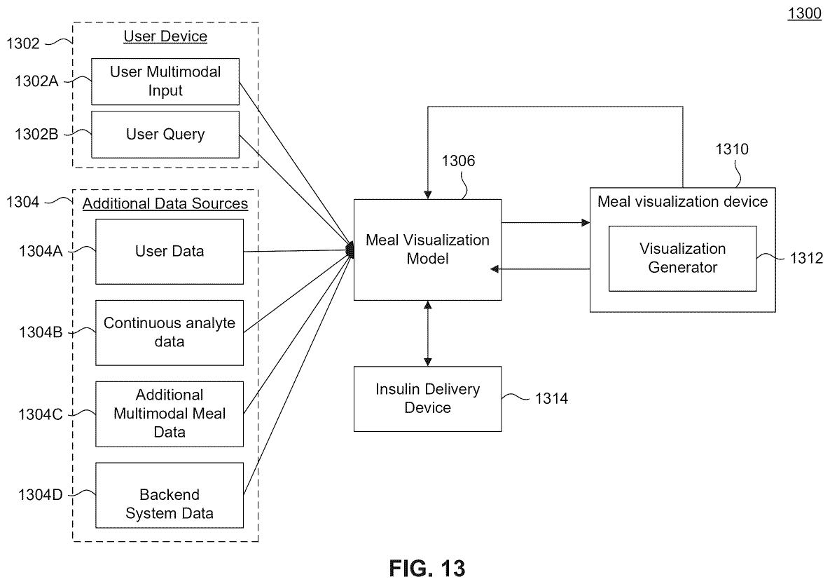

Various aspects of the multimodal meal visualization systems, devices, and methods will now be described in further detail. A particular embodiment of the multimodal meal visualization system, meal visualization system 1300, is depicted in FIG. 13. Reference to the various components of FIG. 13 will be made throughout this section.

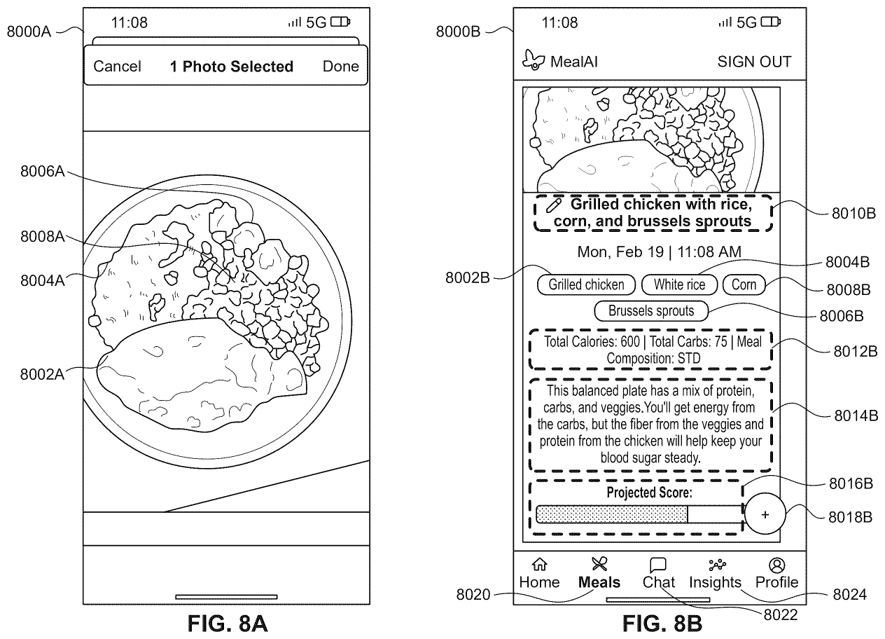

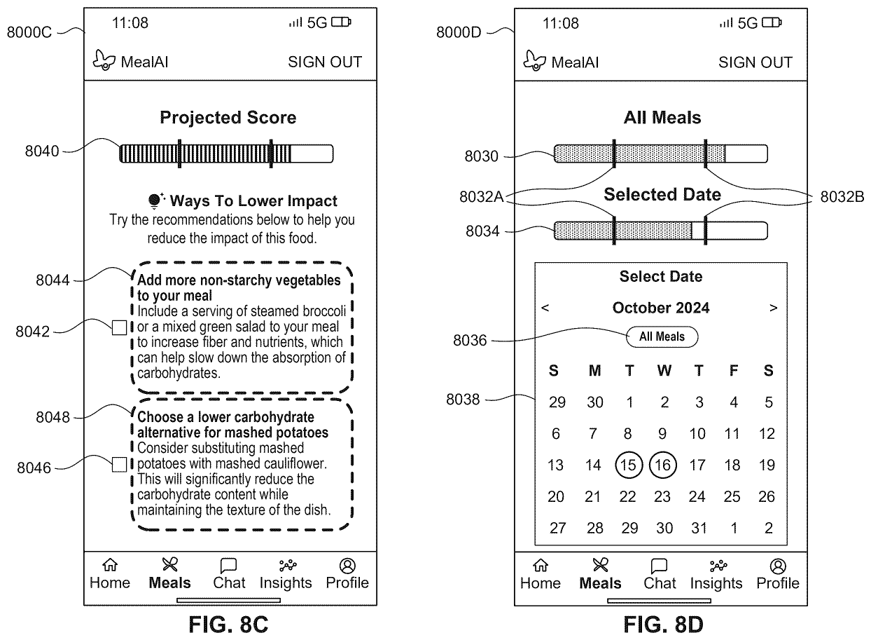

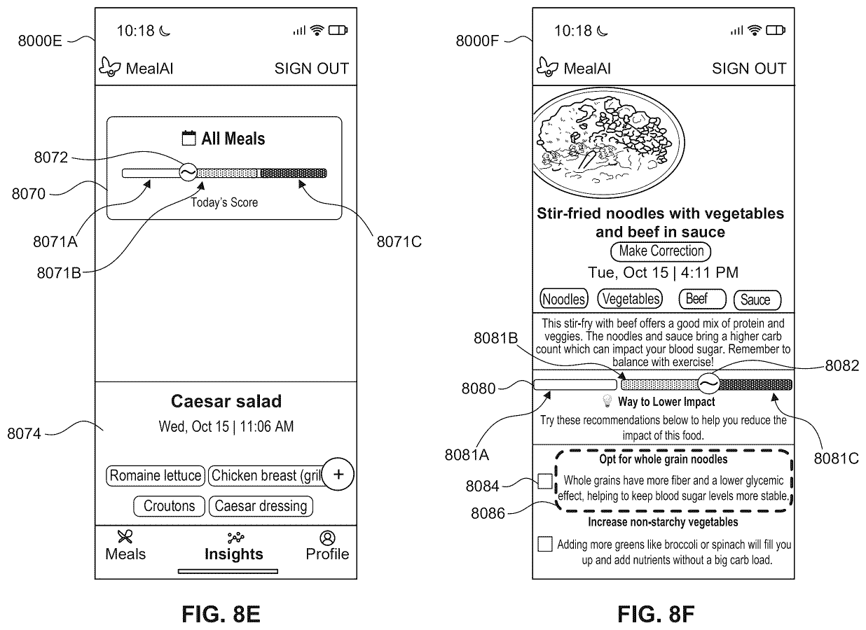

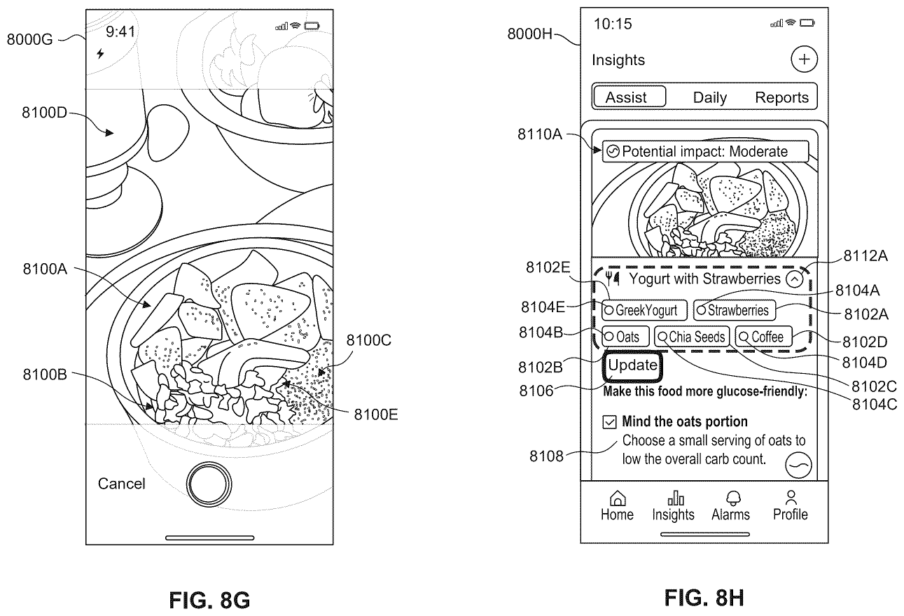

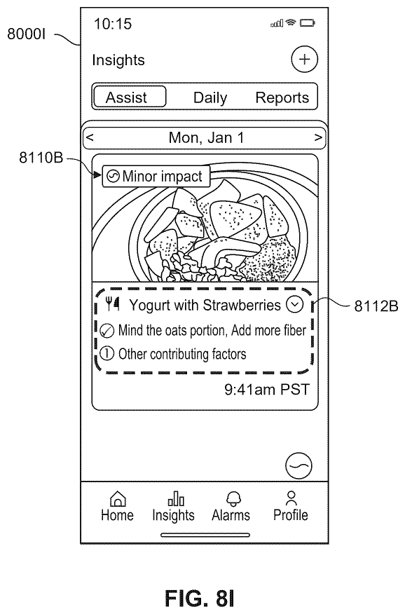

FIGS. 8A-8B are diagrams of a graphical user interface associated with the multimodal meal visualization system for extracting meal information (e.g., metadata) according to an embodiment. In some embodiments, the graphical user interfaces of FIGS. 8A-8B are generated by visualization generator 1312 in response to receiving output from meal visualization model 1306 (e.g., FIG. 13). In some embodiments, the graphical user interfaces of FIG. 8A-8B are displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces.

FIG. 8A depicts a graphical user interface 8000A for receiving multimodal input associated with a meal. In some embodiments, the graphical user interface 8000A may be implemented on a meal visualization device 1310, such as data receiving device 120 or another device as discussed further herein. Graphical user interface 8000A may be implemented as part of a software application installed on the meal visualization device and may be in communication with a meal visualization model 1306 (discussed further below with respect to FIG. 13) that is trained to provide meal information (e.g., metadata) extracted from the multimodal input. As shown in FIG. 8A, graphical user interface 8000A receives image data relating to a meal. The image data may be received from user device 1302, which may also be data receiving device 120 or another device as discussed further herein. Graphical user interface 8000A may also be configured to receive different types of multimodal inputs, such as text data, image data (e.g., still images or video), audio data, or any combination thereof provided by, for example, user device 1302. These additional multimodal inputs are also referred to herein as additional user inputs. These user inputs may be provided to a prompt generator (e.g., prompt generator 1610, prompt library 1645, prompt engine 1651) and provide the inputs to meal visualization model 1306 to extract the meal information (e.g., metadata) from the provided multimodal input. Examples of meal information (e.g., metadata) include meal classification information (e.g., identification of meal components, meal type, meal component type) and meal textual information (e.g., meal information, potential glycemic impact, nutrition information).

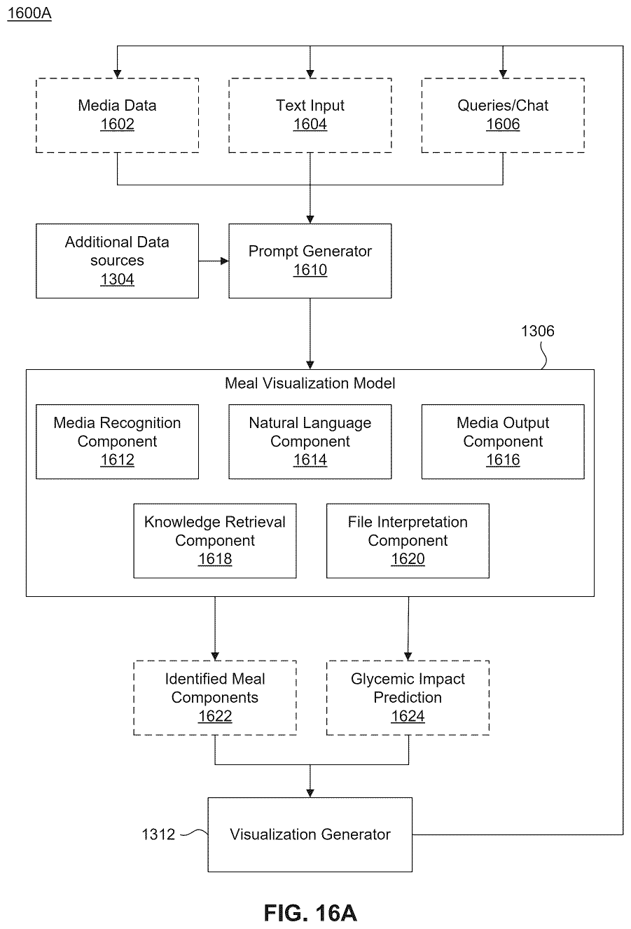

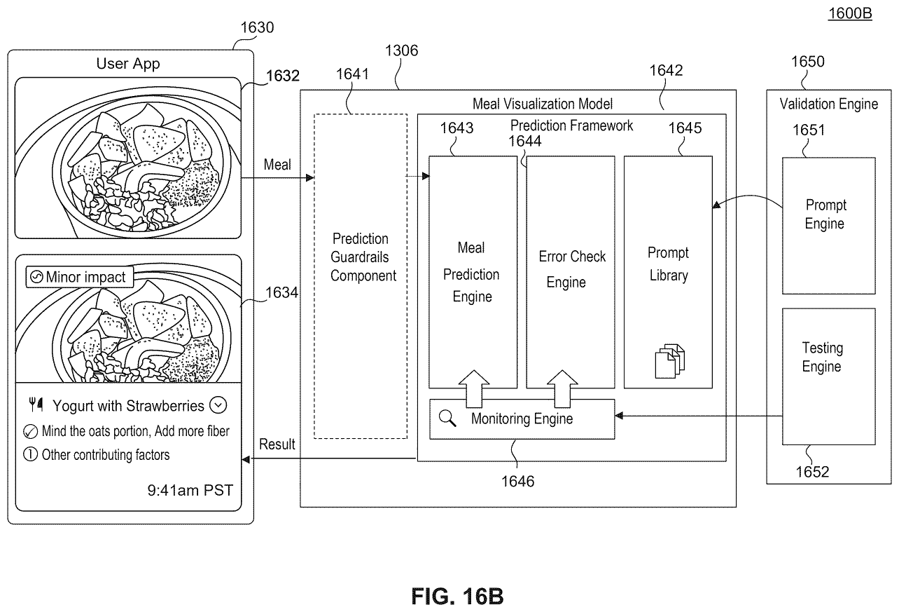

Meal visualization model 1306 may be implemented using one or more machine learning models to address two primary tasks: meal component detection and visualization, and glycemic prediction and recommendation. In some embodiments, pre-trained/pre-existing machine learning model, such as a large language model, especially a multimodal large language model, may be used for the meal visualization model 1306. In some embodiments, a pre-trained/pre-existing machine learning model, such as an ChatGPT-4, Claude, Gemini, or Llama, may be used as the meal visualization model 1306. Further details of a suitable pre-trained/pre-existing machine learning model for meal visualization model 1306 is discussed with respect to FIG. 16A. Meal visualization model 1306 may also include one or more other algorithms for meal component detection and visualization, or glycemic prediction and recommendation. For example, a glycemic impact score (also referred to herein as a glucose impact value and the like) may be determined using the algorithms discussed further herein.

When not using a pre-trained/pre-existing machine learning model, training of meal visualization model 1306 involves several steps. First, datasets are identified and collected for training meal visualization model 1306 to generate outputs that can be converted into visual interface components representative of the provided meal inputs and the predicted impact on the user's health, including their glucose levels. The datasets may originate from various sources, such as databases, files, application programming interfaces (APIs), or real-time sensor feeds. Examples of data in the datasets include but are not limited to user medical information (e.g., glucose levels, medication information, diagnosis information, comorbidities, HbA1C values, historical medical data), population medical information (e.g., population glucose levels, population food logs and diaries), food composition databases that provided nutritional profiles of foods, including data about carbohydrates, sugars, and fiber, user-provided food-diaries and logs, recipe databases, wearable device data, anonymized healthcare databases (i.e. from a healthcare provider (HCP) and/or electronic health records (EHR)), cultural and regional dietary preferences, user feedback data, and food image databases.

The dataset can comprise multiple records, where each record includes one or more features. For example, for user medical information, the records can include physiological measurements such as glucose levels and historical food intake data. Records can be numeric and text-based records obtained from users' medical devices (analyte sensor 104) and health records. Features extracted from these records may include numerical values like average glucose level, other health data such as blood pressure range, and cholesterol readings, and user information (e.g., metadata) such as age, weight, and health conditions. The labels may involve specific health conditions or dietary needs (e.g., diabetic, prediabetic, low cholesterol) to guide recommendation process.

As another example, for food composition databases, each record can represents a specific food item or component within the context of a meal, including its nutritional profile (e.g., carbohydrates, calories, protein, fiber, sugar). Features can include nutrient quantities such as protein, carbohydrates, fats, and vitamins. Labels can indicate dietary categories, such as “high-protein,” “low-carb,” or “vegetarian.”

As another example, for user-provided food diaries, records can consist of textual entries and multimedia (images, video, audio) provided by users, detailing meals intake including portion sizes and meal types. Features can include meal type (e.g., breakfast, lunch, dinner, snack), portion size, and nutritional content extracted from the entries. Natural language processing (NLP) may be used to extract structured information. Labels could the meal components within the user-provided food diaries and the recorded glycemic impact of the meal components on the user.

As another example, for recipe databases, record can consist of meals and a list of ingredients, preparation steps, and associated nutritional values. Features can include ingredients, ingredient quantities, and nutritional breakdown. Labels may specify meal components, meal types (e.g., breakfast, lunch, dinner), meal ethnicities (e.g., Indian, Thai), meal categories (e.g., “low-carb,” “gluten-free”) or suitability for certain health conditions.

Relevant features are selected from the dataset to improve performance of meal visualization model 1306. Feature selection techniques can be used to identify which attributes (features) have the most influence for providing accurate predictions of health impact (e.g., glycemic impact) and for providing accurate detection of meal components within multi-modal data. In some aspects, a combination of filter methods, wrapper methods, and embedded methods may be used. Filter methods are useful for identifying a base dataset based on, for example, correlation analysis to identify relationships between features (e.g., carbohydrate levels) to the target variable (e.g., glycemic impact). A wrapper method is useful for refining the base dataset by ranking features based on their importance in meal component detection and glycemic impact prediction, based on, for example, recursive feature elimination for recursively eliminating redundant features such as overlapping nutrient data. Embedded methods, such as least absolute shrinkage and selection operator (LASSO) or random forest, can be useful for assessing feature importance for wide ranges of data types, such as nutrient information, physiological data. Any combination of these techniques may be utilized to ensure that meal visualization model 1306 is provided with informative and non-redundant data so that training is efficient.

Meal visualization model 1306, when not implemented using a pre-existing multimodal large language model, may be implemented as a hybrid of machine learning models (or sub-models) to address the two primary tasks: meal component detection and visualization, and glycemic prediction and recommendation. For example, the task of meal component detection and visualization requires processing multiple types of inputs (e.g., video, text, image, audio). Meal visualization model 1306 may include multiple sub-models for each type of input and is capable of combining these sub-models into a single system for generating outputs that can be converted into interactive visualizations. In some aspects, existing machine learning models may be adapted for these sub-models.

One example of a sub-model can be a convolutional neural network (CNN) for receiving video inputs to extract visual features and performing object detection to identify meal components within the video. Another example of a sub-model can be natural language processing (NLPs) models for receiving text (structured and unstructured), extracting intent and features from the text input, and identifying keywords related to meal components and glycemic impact. Another example of a sub-model can be a speech-to-text model for converting audio into text, which then be fed into an NLP model, as described above. In aspects where multiple types of input are provided (e.g., video and text, video and audio, or video, text, and audio), meal visualization model 1306 may be perform feature fusion after the sub-models have extracted features from the provided inputs.

For the glycemic prediction and recommendation task, meal visualization model 1306 may utilize another sub-model, such as a regression model for predicting glycemic impacts of a meal. Examples of regression models include linear regression and decision trees. For example, predicting postprandial (after eating) blood glucose levels requires understanding the influence of different meal components, which can comprise a variety of carbohydrates, fats, and proteins, on the user's glucose response. A regression model in meal visualization model 1306 can analyze the relationship between these features and the target glucose levels to provide an accurate glycemic prediction.

The training phase of meal visualization model 1306 can involve feeding a training dataset and allowing meal visualization model 1306, or sub-models thereof, to learn patterns and relationships between the input features and the output target, which are meal component objects, predicted glycemic impact for the particular user, and recommendations associated with the meal for that particular user based on the predicted glycemic impact. During this process, meal visualization model 1306, or sub-models thereof, can iteratively adjust its internal parameters (weights, coefficients, and the like) to minimize the error between its predictions and the actual outcomes.

After training, performance of meal visualization model 1306, or sub-models thereof, may be evaluated using a separate testing dataset that was not seen during training. Evaluating performance of meal visualization model 1306 can require metrics for assessing both classification tasks (e.g., identifying meal components, generating visual outputs) and regression tasks (e.g., predicting glycemic impacts). Regression metrics can include mean squared error (MSE) (e.g., average squared difference between the predicted glucose values and the actual observed values), mean absolute error (MAE) (e.g., a measure of how close the predicted values are to the observed values). Classification metrics can include accuracy (e.g., how well meal visualization model 1306 detects meal components), precision and recall (e.g., how well meal visualization model classifies meal components).

Regardless of whether meal visualization model 1306 includes a pre-trained/pre-existing machine learning model or not, as will be discussed below, new input data can be made available, such as via additional user inputs (e.g., that can be provided via unstructured text in a chat window, that can be provided via additional images or video) as well as recorded actual glycemic impacts. Meal visualization model 1306 may be retrained periodically to maintain or improve its performance. Retraining can be initiated based on temporal intervals, performance thresholds, or changes in the data distribution. Alternatively or additionally, prompts generated by prompt generator 1610 may be adapted to account for the new input data. As a further alternative, prompt templates in prompt library 1645 may be updated to account for new input data.

Output of meal visualization model 1306 may be provided to a front-end interface for generating graphical user interfaces. Exemplary output includes javascript object notation (JSON) files and extensible markup language (XML) files. Output can include any combination of predicted glucose impacts, detected meal components, information about interface layout, components like images and text, user action components like selectable icon 8026, and dynamically generated attributes for each item being displayed in the interface. This data can be used by a visualization front-end, such as visualization generator 1312, for generating graphical user interfaces.

Meal classification information includes meal component identifiers of objects that are detected within the multimodal input. Examples of objects are image objects that are detected via image recognition of the image data, textual objects that are detected via text recognition of image data or within a text query or other textual data, and audio objects that are detected via audio recognition of audio data. For example, graphical user interface 8000A depicts image data comprising meal objects as represented by graphical elements 8002A, 8004A, 8006A, and 8008A. Meal component identifiers provide information about the detected meal objects. For example, a meal component identifier may comprise textual data (e.g., “chicken breast,” “grilled chicken,” “corn,” “white rice,” “brussels sprouts”).

FIG. 8B depicts graphical user interface 8000B that is generated in response to providing multimodal inputs to meal visualization model 1306. Graphical user interface 8000B includes various visual interface components such as a summary text component 8010B, graphical elements 8002B, 8004B, 8006B, and 8008B, nutrition text component 8012B, narrative text component 8014B, glucose impact component 8016B, and input component 8018B. Graphical user interface 8000B may be configured to display, via the various visual interface components, various outputs generated by meal visualization model 1306 in response to the multimodal inputs. Examples of outputs from meal visualization model 1306 may include meal component identifiers associated with the detected objects in the multimodal input and textual data that provides additional information about the detected objects.

The graphical user interface 8000B may be configured to display the outputs of the meal visualization model via the various visual interface components.

Summary text component 8010B may be configured to display summary text composed of the meal component identifiers and arranged into a grammatical format that textually describes the multimodal input.

Graphical elements 8002B, 8004B, 8006B, and 8008B may be configured to separately display meal classification information, such as meal component identifiers, such that the meal component identifiers are distinctly visualized within the graphical user interface 8000B. In some embodiments, the meal component identifiers can be associated with various glucose impact values (or scores), which represents the potential impact that the meal component (e.g., chicken) associated with the meal component identifier has on a user's glucose levels (e.g., will cause the glucose level to increase slightly, to increase gradually, to increase quickly, to increase to a predicted threshold level, etc.). The terms glucose impact value and glycemic impact score are used interchangeably and represent a numerical value that is used to adjust an overall glucose impact prediction for a user. The association of the meal component identifier may be predetermined, determined dynamically (e.g., for each particular user associated with meal visualization device 1310), or a combination of both. For example, certain meal components may be predetermined to have a glucose impact value (e.g., white rice associated with a rapid increase value or a high glucose value) within the software application installed in the meal visualization device 1310. As another example, the association between the meal component and the glucose impact value may be determined and/or updated dynamically on a per-user (e.g., user of the meal visualization device) or a per-cohort (e.g., males, females, males 17 or under, males over 17, females 17 or under, males with a diabetes, persons with a recent HbA1C value above a threshold, etc.). In some embodiments, meal visualization device 1310 may generate meal-specific predictions for each glucose impact value score based on glucose and meal records from a cohort of other users that are associated with the current user of meal visualization device.

In some embodiments, to update the glucose impact value associations, the software application providing graphical user interface 8000B may be configured to store user meal and glucose history. In such embodiments, the software application may be configured to be connected with a continuous analyte monitor, such as continuous glucose sensor (e.g., analyte sensor 104 or sensor control device 102) and receive analyte data that is based on measured analyte levels and time-coded with meal information. The analyte data and the meal information (e.g., meal component identifiers) may be provided to meal visualization model 1306, which is configured to identify the associations between the meal component identifiers and the glucose impact values. For example, meal visualization model 1306 may be configured to identify associations for each meal component identifier (e.g., grilled chicken identifier, broccoli identifier, white rice identifier) based on the analyte data. In this manner, the identified associations are personalized based on the analyte data of a user. Graphical user interface 8000B may include another visual component that displays the stored analyte data with the meal information.

In some embodiments, the meal classification information may include color-coded tags associated with each glucose impact value (e.g., red or orange tags associated with a high glucose impact value, yellow tags associated with a moderate glucose impact value, and green tags associated with a low glucose impact value). The graphical user interface 8000B may be further configured to display the meal component identifiers based on the color-coded tags that are associated with the respective glucose impact values for each meal component identifier.

It is understood that color-coded tags are merely an exemplary visualization indicator that may be used to visually distinguish between different glucose impact values. For example, different shapes, different icons, different shading, different fonts, etc., may be associated with each glucose impact value, and the meal component identifiers may be displayed according to any of these visualization indicators.

Glucose impact value can be contextual based on current user health information, such as their current glucose value, whether the user is diabetic (including type), user meal preferences, insulin dosing information, medical history, medical conditions (e.g., comorbidities), dietary preferences, and user meal history. Contextual glucose impact values are also referred to herein as personalized glycemic impact scores. Glucose impact values that are not personalized are referred to herein as generalized glycemic impact scores. Meal visualization model 1306 can receive, as inputs, the meal classification information, and a current glucose value associated with the user, and generate the glucose impact value in accordance with the calculated impact of the meal based on the current glucose value. For example, meal visualization model 1306 may calculate a meal to have a high glucose impact when the current glucose value of the user is above a certain threshold value or metric (e.g., “high”) but the same meal may be calculated to have a medium glucose impact when the current glucose value is below a certain threshold value or metric (e.g., “low” or “normal”).

An overall glycemic impact score, also referred to herein as a total glycemic impact score, or composite glycemic impact score, may be generated based on one or more of the glucose impact values generated for the meal components. For example, the respective glucose impact values may be provided as input to a meal visualization model (e.g., meal visualization model 1306) that is prompted or otherwise trained to provide as output a composite glycemic impact score, which reflects a predicted glycemic impact on the user. In some aspects, user medical information, such as current glucose value, historical glucose values, historical glycemic impact, are also provided as an input within a predefined prompt template for generating a composite impact score that is also personalized based on the detected meal components and the user medical information. Alternatively, composite impact score may be generalized. Glycemic impact scores which are not composite impact scores may be referred to herein as individual impact scores since these correspond to a single meal component.

FIG. 8B includes four meal component identifiers, i.e. graphical elements 8002B, 8004B, 8006B, and 8008B. It is understood that more or fewer meal component identifiers may be displayed than shown in FIG. 8B. In some embodiments, the number of meal component identifiers is based on the number of detected meal objects (e.g., components or ingredients) within the provided multimodal inputs. In some embodiments, the meal component identifiers may be manually changed by the user (e.g., removed, added, changed), and the system would determine a glycemic impact score for the updated meal components and/or for the overall meal.

Nutrition text component 8012B may be configured to display nutrition information associated with each meal component identifier. The nutrition information may be predefined or determined dynamically by meal visualization model 1306. For example, there may be preset nutrition information for “grilled chicken” that can be utilized and displayed as part of nutrition text component 8012 (e.g., when the meal component identifier associated with “grilled chicken” is determined by meal visualization model 1306). In some embodiments, meal visualization model 1306 may be configured to determine the nutrition information based on the multimodal inputs. For example, meal object 8002A may be detected by meal visualization model 1306 using an image recognition process. The image recognition process may be configured to further identify an estimated portion size of meal object 8002A and meal visualization model 1306 may be configured to convert the estimated portion size to a corresponding nutrition text component 8012B. Meal visualization model 1306 may be further configured to estimate the nutrition information based on the estimated portion size and the meal component identifiers detected within the multimodal input.

For example, meal visualization model 1306 may detect meal components/objects as provided by graphical elements 8002A, 8004A, 8006A, and 8008A and also estimate the respective portion size for each detected meal object. Meal visualization model 1306 may be configured to determine the nutrition information (e.g., calories, carbohydrates, protein, fat, potential glycemic impact value) based on both the meal object identifiers (e.g., graphical elements 8002A, 8004A, 8006A, and 8008A) and the estimated portion sizes for each. For example, glycemic impact value may be represented by a range of values and potential impacts based on other user information. That is, a meal can have a different glycemic impact for one user compared to another, which can be reflected in the range of values for potential glycemic impact. In some examples, meal object identifiers (e.g., graphical elements 8002A, 8004A, 8006A, and 8008A) may include a glycemic visual indicator for the meal object's predicted meal impact (e.g., red or orange color for high glycemic impact, yellow or green color for medium glycemic impact, yellow or green color for low glycemic impact). In some examples, the glycemic visual indicators may display a gradient representing the meal object's predicted meal impact where the indicator for higher impact food are more opaque than lower impact foods. Meal visualization model 1306 may the provide the determined nutrition information to graphical user interface 8000B for display.

Narrative text component 8014B may be configured to display meal textual information generated by meal visualization model 1306 based on the meal classification information (e.g., meal component identifiers). The meal textual information differs from summary text in that the meal textual information is generated text provided by meal visualization model 1306 and formatted into a narrative format (e.g., sentences).

In some embodiments, the meal textual information may be generated based solely on meal component identifiers. In this embodiment, different meal visualization devices (e.g., meal visualization device 1310) for different users could receive the same meal textual information based on the same meal component identifiers. For example, meal component identifiers for chicken, rice, and beans could result in the same meal textual information providing narrative description for the meal component identifiers.

In some embodiments, the meal textual information may be generated based on meal component identifiers and additional user input, which may include one or more of, user provided input (e.g., via a user input box provided by graphical user interface 8000B), user medical history (e.g., analyte history), and user analyte data (e.g., current analyte level). In this embodiment, different meal visualization devices for different users could result in different meal textual information based on the same meal component identifiers.

Glucose impact component 8016B may be configured to display a glucose impact visualization associated with the combination of glucose impact values associated with the meal component identifiers (e.g., graphical elements 8002B, 8004B, 8006B, and 8008B) for the provided multimodal input. Glucose impact value is also referred to herein as a glucose impact score, glycemic impact score, glycemic impact value, glycemic impact prediction, and the like. Glucose impact component 8016B may be configured to provide a projected or predicted score (i.e., a glycemic impact prediction, for the meal based on the meal component identifiers and user health information. The glucose impact visualization is configured to quantify the impact of the identified meal and its constituent meal component identifiers based on both meal information (e.g., nutrition information, portion sizes), user health information (e.g., current glucose level, exercise information, insulin dose information, medication information), and user history (e.g., whether the user is diabetic, meal preferences, food restrictions). Glucose impact component 8016B may display the predicted score for the meal as a bar graph as shown in FIG. 8B. In other examples, glucose impact component 8016B may be a numerical value, color coded indicator, etc. Other visualizations for glucose impact component 8016B may not display a number value or score.

In some embodiments, a pre-trained/pre-existing machine learning model, such as an ChatGPT-4, Claude, Gemini, or Llama, may be used as the meal visualization model 1306 to determine the glycemic impact value. In other embodiments, the meal visualization model 1306 may have a sub-model to generate the glucose impact values.

In either embodiment, the glycemic impact value (e.g., glycemic impact prediction 1624) may be generated using an algorithm provided within the prompt (e.g., generated by prompt generator 1610, prompt library 1645, or prompt engine 1651). The algorithm may be embedded in the prompt and further include placeholders into which inputs may be inserted for generating the glycemic impact value. For example, the algorithm may define inputs as meal components, nutritional values (e.g., carbohydrates, fiber), and user information (e.g., current glucose value, glucose history). Meal visualization model 1306 may then generate glycemic impact prediction 1624 based on the provided algorithm and associated inputs.

In some embodiments, the prompt template may include additional instructions for causing the meal visualization model 1306 to validate glycemic impact prediction 1624, such as by comparing the glycemic impact prediction 1624 to a history of glycemic impact predictions (e.g., by accessing a database).

When using a sub-model (i.e. not a pre-trained/pre-existing machine learning model) to generate the glucose impact values, to generate this information, meal visualization model 1306 may be trained using a pre-compiled dataset containing meal information and meal components, and their baseline potential impact on glucose values. This pre-compiled dataset may be provided based on user cohort characteristics (e.g., any combination of gender, sex, age, medical history), meal type (e.g., breakfast, lunch, dinner, and snack) and include the impacts (i.e., real world or simulated) on glucose values. During training, meal visualization model 1306 can learns to recognize patterns of the meal information and components on glucose values and identifying relationships between the glucose impact and the user cohort characteristics.

When the meal and its components are identified, meal visualization model 1306 can extract feature vectors of the meal and its components and compare them to those of meals and components from the training set. This comparison can be performed using similarity metrics, such as cosine similarity, which measure the degree of alignment between the identified meal and the meals from the training data. In some embodiments, the more closely the detected meal aligns with meals from the training sets, the detected meal is assigned a corresponding total glycemic impact score and each individual meal component can be assigned an individual glycemic impact score.

Meal visualization model 1306 may continuously refine the output based on additional user interactions. As meal visualization model 1306 continues to receive multimodal inputs—menu images, food images, meal images, audio data, user input, user medical history—this interaction data is fed back into the meal visualization model 1306. This feedback allows meal visualization model 1306 to iteratively improve its graphical user interfaces and associated visual components over time, adapting the generated components based on evolving activity and preferences.

As noted above, meal visualization model 1306 provides the predicted glucose impact values (scores) as data within output (e.g., JSON file) to visualization generator 1312 which parses the output and generates graphical user interfaces based on the data, metadata, and other information provided within the output.

Meal visualization model 1306 may be configured to determine the glucose impact visualization based on directly on the glucose impact value. In the embodiment shown in FIG. 8B, glucose impact component 8016B is implemented as a bar graph that reflects the glucose impact value for the meal component identifiers. Another visualization for glucose impact component 8016B may include a trend graph depicting the predicted changes to the user's glucose level based on the glucose impact value. And another visualization may be a numerical text that states the glucose impact value.

Another example of a glucose impact visualization is color-coding based on the glucose impact values. For example, glucose impact values below a low threshold amount (i.e., representing a low impact) may be color-coded green, above the low threshold amount but below a high threshold amount (i.e., representing a medium or moderate impact) may be color-coded yellow, and above the high threshold amount (i.e., representing a high impact) may be color-coded red, orange, or another color. Each of graphical elements 8002B, 8004B, 8006B, and 8008B may then be color-coded based on the predicted glucose response for each corresponding meal component (e.g., 8002B can be green, 8004B can be orange, 8006B can be orange, and 8008B can be yellow). It should be understood that each of the low impact, moderate impact, and high impact visualizations may be any other colors and/or two or more of the visualizations may be the same color (e.g., low impact and high impact visualizations may be the same color). It is understood that the number of thresholds within the glucose impact visualization may vary to accommodate different numbers or types of thresholds, such as low, medium and high or low, low-moderate, moderate, moderate-high, and high. In some embodiments, the threshold amounts and color-coding may be personalized to each individual user such that the same meal may be color-coded differently for each user. The threshold amounts may be adjusted over time based on user history, user activity, and other health inputs associated with the user so that the threshold amounts do not remain static over time. In some examples, the threshold amounts and color coding may be adjusted by the user. In some aspects color-coding may be applied as tags or other visual indicators (such as an icon, a dot, or dash, a line) within graphical elements 8002B, 8004B, 8006B, and 8008B.

Another embodiment of glucose impact component 8016B is a gauge representation of the projected score of the entire meal (including the individual components). The gauge representation can be configured to represent low, medium, and high impact through a gauge meter. The gauge meter may also be color-coded as described above. In some examples, the gauge meter may display a numerical number for the projected impact score of the team (e.g., between 0 or 1 (low) and 100 (high), between 0 or 1 (low) and 10 (high)).

FIG. 8C depicts an exemplary graphical user interface 8000C configured to display another embodiment of a glucose impact component 8040 that is dynamically configurable based on additional user input. For example, graphical user interface 8000C may include a first selectable option 8042 associated with a first recommendation component 8044 and a second selectable option 8046 associated with a second recommendation component 8048. FIG. 8C shows a check box for first selectable option 8042 and second selectable option 8046, but other types of selectable option may be used. In general, the phrases “selectable option” and “selectable object” are used interchangeably herein. However, it should be appreciated that other numbers of selectable options or objects may be shown. As with other graphical user interfaces discussed herein, the graphical user interface of FIG. 8C may be displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces.

In some aspects, selection of either first selectable option 8042 and second selectable option 8046 does not require providing additional inputs to meal visualization model 1306. For example, first selectable option 8042 is linked to data within the output (e.g., JSON file) provided by meal visualization model 1306 and selection of first selectable option 8042 can cause visualization generator 1312 to identify and retrieve the linked data and update graphical user interface 8000C based on the retrieved linked data. As one example, first selectable option 8042 may be linked to data that updates the visual depiction of glucose impact component 8040 where a higher glycemic impact score would result in increasing the shading of glucose impact component 8040 and a lower glycemic impact score would result in decreasing the shading of glucose impact component 8040. Other methods for updating glucose impact component 8040 are possible for reflecting a change in the predicted glycemic impact score. For example, as discussed in FIG. 8E, an indicator icon such as indicator icon 8072 may be moved along the glucose impact component 8040. Instructions for performing this update may be provided as part of the output of meal visualization model 1306.

As another example, second selectable option 8046 is linked to other data within the output provided by meal visualization model 1306. Selection of second selectable option 8046 can cause visualization generator 1312 to identify and retrieve the linked data and update graphical user interface 8000C based on the retrieved linked data. As with first selectable option 8042, selection of second selectable option 8046 can also result in updates to graphical user interface 8000C based on the data from the output of meal visualization model 1306. As previously noted, output from meal visualization model 1306 may include additional instructions for updating glucose impact component 8040 based on the selection of second selectable option 8046, without having to provide those additional inputs to meal visualization model 1306 or from another external source.

In the embodiment shown in FIG. 8C, first recommendation component 8044 and second recommendation component 8048 may be associated with recommendations for lowering glycemic impact. Any combination of first selectable option 8042 or second selectable option 8046 may be selected which can result in updating glucose impact component 8040. Graphical user interface 8000C is not limited to number of selectable options displayed. Any number of selectable options may be displayed (e.g., as instructed by a prompt from prompt generator 1610, prompt library 1645 or prompt engine 1651) and any combination of selectable options may be selected which can result in updating glucose impact component 8040 based on instructions in output generated by meal visualization model 1306. Text for first recommendation component 8044 and second recommendation component 8048 may also be retrieved from the output and populated into first recommendation component 8044 as part of the visualization process by visualization generator 1312. Other configurations of graphical user interface 8000C are possible (e.g., additional selectable options, additional recommendation components) and are not limited to the components and layout depicted in FIG. 8C.

In embodiments, recommendation components, such as first recommendation component 8044 and second recommendation component 8048 can be selected from a predefined output library. Meal visualization model 1306 can be configured to select predefined visualizations, such as from an visualization library, rather than generate its own visualizations and text via an large-language model dynamically for each prompt, based on the glucose impact value and user information. In embodiments, prompts from prompt generator 1610, prompt library 1645 or prompt engine 1651 can be configured to force meal visualization model 1306 to select predefined visualizations from a visualization library, such as predefined text for different conditions defined by the glucose impact value and user information. For example, a predicted output library may include predefined text (e.g., “Mind the oats portion, add more fiber,” “minor impact,”) and visualization components (e.g., various visual interface components such as a summary component 8112A, meal component elements 8102A-E, score indicators 8104A-E, input component 8106, recommendation component 8108, glucose impact component 8110A). Other examples of predefined output can include recommendations with placeholder variables, which can be populated based on meal and user information, recommendations organized based on category of the meal or meal component (e.g., high fiber, high protein, high carbohydrate), and recommendations based on user information, such as how often a user followed or did not follow a particular recommendation.

FIG. 8D depicts an exemplary graphical user interface 8000D configured to display other exemplary embodiments of glucose impact components for different time windows, such as glucose impact component 8030 and glucose impact component 8034. Glucose impact component 8030 is configured to display a calculated glycemic impact score for the user for all recorded meals for a particular time period (e.g., a day, a week, a month, a year). Display of glucose impact component 8030 may be triggered via selection of a selectable icon 8036 within graphical user interface 8000D. In some aspects, selection of selectable icon 8036 triggers access to an output file (e.g., a JSON file) from meal visualization model 1306, without having to provide additional input to meal visualization model 1306. Upon selection of selectable icon 8036, visualization generator 1312 can be configured to retrieve the relevant information, such as the glycemic impact score for the time period associated with selectable icon 8036.

Glucose impact component 8034 is configured to display a predicted glucose score for the user for all recorded meals for another particular time period, that is selectable using a time period selector 8038 (e.g., a calendar with dates). Time period selector 8038 is configured to display selectable time periods (e.g., dates) where predicted glucose scores are available for display (e.g., available within the output file provided by meal visualization model 1306). In some embodiments, glucose impact component 8034 may be configured to display an actual glycemic score in addition to or in place of the predicted glucose score for a particular time period. Visualization generator 1312 can be configured to retrieve actual glycemic scores as needed, based on data from output provided by meal visualization model 1306 or based on user preferences. In some embodiments, time period selector 8038 may be configured to highlight one or more specific time periods (e.g., days) to indicate when a user implemented a recommendation (e.g., when selecting selectable icon 8036) or when a predicted glucose score was accurate (e.g., by comparing against the actual glycemic score).

In some embodiments, selection of selectable icon 8036 will cause data from all time periods to be retrieved from output (e.g., a structured output file) provided by meal visualization model 1306. In this manner, selection of selectable icon 8036 can cause glucose impact component 8034 to be visually updated on the graphical user interface 8000D for all time periods.

Glucose impact component 8030 and glucose impact component 8034 can be figured with threshold indictors, such as first threshold indicator 8032A and second threshold indicator 8032B for indicating various threshold values associated with the glycemic impact scores associated with each impact component. For example, first threshold indicator 8032A and second threshold indicator 8032B may be configured to indicate safe, potentially unsafe, and unsafe (or low, medium, and high) glycemic impact scores. Other configurations of graphical user interface 8000C are possible and are not limited to the components and layout depicted in FIG. 8D.

Returning to FIG. 8B, input component 8018B may be configured to receive user input (e.g., a touch-based input on a display of meal visualization device) and configured to display additional visualizations based on the received user input. Examples of additional visualizations include graphical user interface 8000A for receiving additional image data as multimodal input or a text box for receiving textual user input for updating or changing the meal component identifiers. Meal visualization model 1306 may provide updated outputs based on the additional user inputs from any graphical user interface (e.g., graphical user interface 8000A, 8000B).

One example of image data includes unstructured meal information, such as an image of a menu at a restaurant or an image of a food available in a store (e.g., grocery store). Meal visualization model 1306 is configured with image recognition and natural language processing capabilities (e.g., as sub-models or otherwise) for processing the image data and textual data within the image data to identify meals and meal objects (e.g., individual meal components or ingredients), and generating a graphical user interface with interactive visual elements that are based on the detected meals and meal objects. In the example of an image of a menu, meal visualization model 1306 may use image recognition capabilities to identify an object in the image as menu with textual data, and based on this identification can trigger natural language processing for identifying the textual data within the image. In this example, the textual data includes description of different menu items which meal visualization model 1306 can use to generate a user interface with each of these menu items associated with an interactive visual element. The interactive visual elements may display information about each of the identified menu items and their components, as well as generated text about the potential impact of the menu item on the user's glucose values. In this manner, meal visualization model 1306 may have access to user health information in order to generate the textual data that populates each of the interactive visual elements.

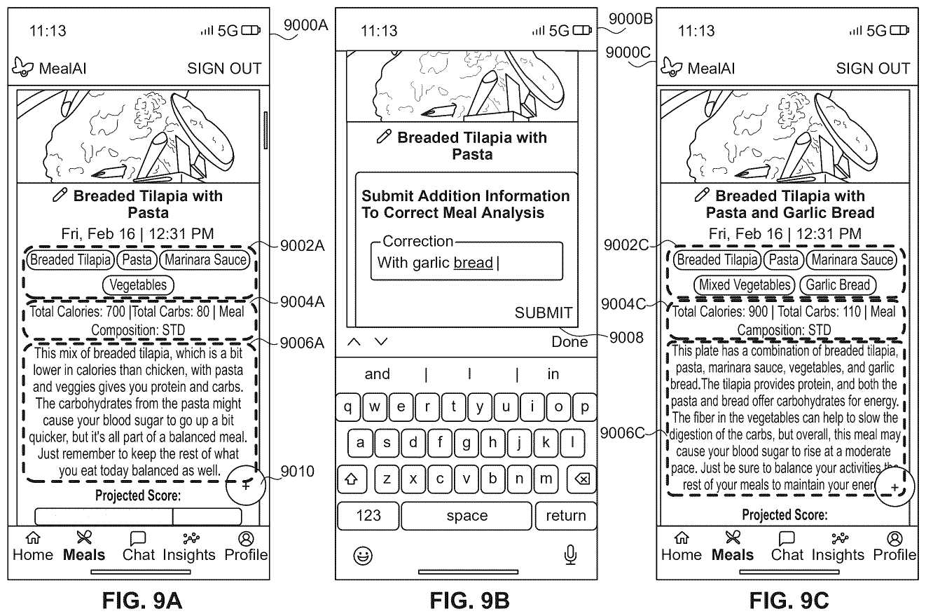

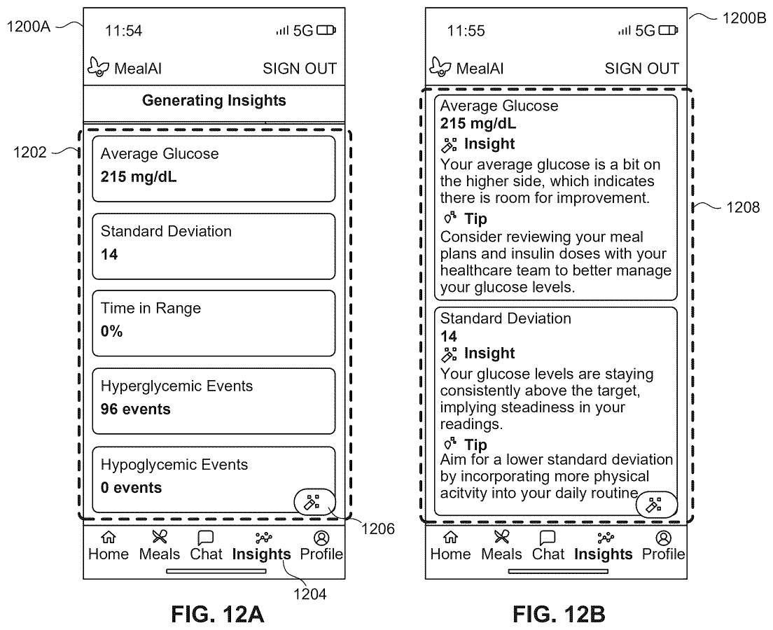

Graphical user interface 8000B may further include visual interface components, such as icons 8020, 8022, and 8024, for accessing additional multimodal features of meal visualization model 1306. Each icon may be configured to provide visual access to different outputs meal visualization model 1306. For example, icon 8020 may be configured to access the meal detection function (e.g. any of the graphical user interfaces of FIGS. 8A-8I or FIGS. 9A-9C), icon 8022 may be configured to access an interactive chat function, and icon 8024 may be configured to access an insights function (e.g. any of the graphical user interfaces of FIGS. 12A-12B).

As with other graphical user interfaces discussed herein, the graphical user interface of FIG. 8C may be displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces.

FIG. 8E depicts exemplary graphical user interface 8000E which is an alternative embodiment of graphical user interface 8000D. Graphical user interface 8000E includes glucose impact component 8070 that can be dynamically configurable and updateable to illustrate an predicted glycemic impact of meals over a predefined period of time (e.g., one day, two days, one week). By “dynamically configurable” it is meant that the glucose impact component 8070 may be personalized to the user. By “updatable” it is meant that the glucose impact component 8070 may be updated in response to the user interacting with the graphical user interface 8000E, for example adding further meal components using component 8074. Graphical user interface 8000E includes an indicator icon 8072 that visually indicates the predicted glycemic impact value for the selected period of time. Graphical user interface 8000D also can include a version of graphical user interface 8000B for depicting a meal, meal components, or other meal related information for a particular period of time. Graphical impact component 8070 may be divided into different visual regions 8071A, 8071B, and 8071C for depicting different glycemic impact thresholds that can be specific to the user. The visual regions 8071A, 8071B, and 8071C may be color-coded to depict different levels of glycemic impact.

FIG. 8F depicts exemplary graphical user interface 8000F which is an alternative embodiment of graphical user interface 8000C and/or 8000D. Graphical user interface 8000F can include glucose impact component 8080 and indicator icon 8082 for displaying a dynamically configurable and updateable visualization of a predicted glycemic impact, in combination with a selectable option 8084 and recommendation component 8086. Glucose impact component 8080 may include visual regions 8081A, 8081B, and 8081C, to reflect different glycemic impact threshold that can be specific to the user. For example, the different thresholds may reflect different levels of glycemic impact as defined by particular thresholds (e.g., low, moderate, and high). These threshold may be specific to each user (i.e., different values) and can be provided as output by meal visualization model 1306 that is generated based on instructions and other inserted input in the prompt that is provided by prompt generator 1610. Prompts may alternatively be provided by a prompt library 1645 and/or a prompt engine 1651.

Selectable option 8084 can operate similarly to selectable option 8044 and selectable option 8046, which can cause glucose impact component 8080 to be dynamically updated based on the glycemic impact associated with the recommendation component 8086. The output provided by meal visualization model 1306 can include this information (e.g., in the structured output file) and selection of selectable option 8084 can cause visualization generator 1312 to retrieve the linked data (i.e., from the structured output file) without having to submit new inputs to meal visualization model 1306.

Recommendation component 8086 may be associated with one or more proposed modifications to the detected meal components, such as replacing a meal component (e.g., “noodles”) with another meal component (e.g., “whole grain brown rice”) that will reduce the glucose impact represented by glucose impact component 8080. In embodiments, recommendation component 8086 is configured to include a user selectable visual element (e.g., a checkbox, a radio button) for selecting and/or deselecting a recommendation that will result in updating the meal components of the meal and/or the predicted glycemic impact of the meal based on the updated meal component. In embodiments, selection of recommendation component 8086 can cause a follow-up request to be transmitted to the meal visualization model 1306 for generating an updated graphical user interface with updated interactive visual elements based on the updated meal components (e.g., whole grain brown rice instead of noodles). In other embodiments, selection of recommendation component 8086 can result in an automatic change to the updated graphical user interface (e.g., without transmitting a follow-up request) based on predetermined information regarding how recommended changes could modify a prediction, for example as stored in the structured output file. For example, selecting certain meal choices can automatically result in lowering a glucose impact for a meal.

FIGS. 8G-I depict alternative embodiments of graphical user interfaces for displaying customized visual interface components based on multimodal inputs, including image data and user inputs.

FIG. 8G depicts an exemplary graphical user interface 8000G for receiving multimodal input associated with a meal. In embodiments, graphical user interface 8000G is an alternative embodiment of graphical user interface 8000A, which depicted receiving multimodal input via user input of a captured images (e.g., from an image library stored on a meal visualization device 1310 or other imaging device). Graphical user interface 8000G is configured to receive multimodal input via an image and/or video capture feature of an application installed on a meal visualization device 1310, such as data receiving device 120, or other imaging device. As with other graphical user interfaces discussed herein, the graphical user interface of FIG. 8G may be displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces.

In some embodiments, graphical user interface 8000G may be implemented as part of a software application installed on the meal visualization device 1310 and may be in communication with a meal visualization model 1306 that is trained to provide meal information (e.g., metadata) extracted from the multimodal input. Examples of multimodal input include any combination of image data, audio data, and video data. In some embodiments, input may be provided via one or more sensors of the meal visualization device, which can include ambient sensors (e.g., temperature, light), an accelerometer, a gyroscope, a barometer, and a lidar scanner. These additional sensors can be used to provide additional information about the meal and/or user. As one example, the lidar scanner can be utilized to capture meal size information, which can be combined with image data to provide additional input for a prompt template. Graphical user interface 8000G provides an image capture interface for capturing meal data which may include image data, audio data, and/or video data. In embodiments, the meal data may include any number of meal components 8100A-E.

Meal visualization model 1306 is configured to receive the multimodal input provided from graphical user interface 8000G and perform meal component detection on the multimodal input. In embodiments where multimodal input is implemented as including image data, meal visualization model 1306 is configured to utilize image recognition techniques on the image data to detect one or more meal components. Prompt templates can be utilized for inputting the multimodal input into meal visualization model 1306. In embodiments, prompt templates can be customized with instructions to cause meal visualization model 1306 to provide output (e.g., detected meal components) in a standardized format. Prompt templates can be so configured to ensure that consistent output in an expected format from the meal visualization model 1306 even if the multimodal input is different. Output that can be configured by prompt templates include detected meal components, a confidence score associated with the detected meal components, a glycemic impact score associated with each detected meal component, an overall glycemic impact score associated with the combination of meal components, and/or recommendations generated based on the detected meal components. Any other interface components discussed herein may also be configured by prompt templates.

In embodiments, prompt templates can be configured with instructions for formatting output from meal visualization model 1306 to include identifiers or tags for categorizing output (e.g., detected meal components, confidence score, glycemic impact score, and recommendation text) such that each part of output can be linked to corresponding visual components for display (e.g., identifier linking a detected meal component with a meal component element).

FIG. 8H depicts an exemplary graphical user interface 8000H that is generated in response to providing multimodal inputs to meal visualization model 1306. In embodiments, graphical user interface 8000H is implemented as an alternative embodiment to graphical user interface 8000B. Graphical user interface 8000H can be generated to display output from meal visualization model 1306. In embodiments, graphical user interface 8000H is configured with various visual interface components such as a summary component 8112A, meal component elements 8102A-E, score indicators 8104A-E, input component 8106, recommendation component 8108, glucose impact component 8110A. Graphical user interface 8000H may be configured to display, via the various visual interface components, various outputs generated by meal visualization model 1306 in response to multimodal inputs. In embodiments, meal component elements 8102A-E are visual representations of meal component identifiers generated by meal visualization model 1306 in response to detected objects in the multimodal input.

Summary component 8112A can be configured to display visualizations associated with the output from meal visualization model 1306. In embodiments, the output includes detected meal components within the multimodal input and the visualizations can be configured as meal component elements representative of the detected meal components. For example, meal component elements 8102A-E exemplary and correspond to meal components 8100A-E, as detected by meal visualization model 1306. For example, meal component element 8102A is a visual component representing meal component 8100A, meal component element 8102B is a visual component representing meal component 8100B, meal component element 8102C is a visual component representing meal component 8100C, meal component element 8102D is a visual component representing meal component 8100D, and meal component element 8102E is a visual component representing meal component 8100E. Other numbers of meal components and meal components elements may be used depending on the number of meal components present (e.g. in the image data and/or subsequently added by the user).

Meal component elements 8102A-E may be configured with score indicators 8104A-E. In embodiments, output from meal visualization model 1306 includes values associated with each detected meal component; in embodiments, a prompt template may include instructions for causing meal visualization model 1306 to generate an impact value representing the potential general impact (i.e., not necessarily personalized to a user) of the detected meal component on a glucose level. In some embodiments, the predictions may involve a combination of general impact and personalize impact information including recommendations. In some embodiments, the impact value is also based on a person's current glucose level and/or historical glucose data. For example, a high impact value (compared to a threshold number) can indicate that the detected meal component may have a high impact on a general glucose level, a medium impact value can indicate a potential medium impact, and a low impact value can indicate a potential low impact. Score indicators 8104A-E can be configured to display visual indicators representing the values associated with each detected meal component. Examples of such visual indicators include any combination of coloring, shapes, shading, and text. For example, score indicators 8104A-E may be configured to display different colors representing different threshold ranges for high impact values (e.g., red or orange), medium impact values (e.g., yellow), and low impact values (e.g., green).

Input component 8106 may be configured to operate similarly to input component 8018B to receive additional user input (e.g., a touch-based input on a display of meal visualization device) and configured to update summary component 8112A based on the received user input.

Examples of additional visualizations include graphical user interface 8000A for receiving additional image data as multimodal input or a text box for receiving textual user input for updating or changing the meal component identifiers. Meal visualization model 1306 may provide updated outputs based on the additional user inputs from any graphical user interface (e.g., graphical user interface 8000A, 8000B).

In embodiments, input component 8106 can be configured as a selectable button or text for receiving additional input regarding the meal component elements 8102A-E. For example, input component 8106 can be configured to provide an input screen for revising (e.g., adding, removing) meal component elements 8102A-E. This option allows corrections to the meal component elements 8102A-E such as if meal visualization model 1306 incorrectly identified any meal components in the multimodal input. (e.g., allows the user to add, remove, or change meal components manually). In embodiments, changes to suggested meal components can be provided as inputs to meal visualization model 1306 to generate an updated meal impact score based on the changes to any suggested meal components.

As another example, input component 8106 can be configured to provide an input screen for providing additional multimodal input to concatenate separate meals that occur within a predetermined time period in order to determine a single glycemic impact of the meals. For example, a user may eat separate meals or snacks within a predetermined time frame (e.g., within 30 minutes, within 1 hour, within 2 hours). Meal visualization model 1306 can be configured to receive separate multimodal inputs (e.g., image data) associated with different meals and further configured to concatenate the meal components in order to determine the glycemic impact from the meals (rather than determining separate glycemic impacts for each meal). In such embodiments, input component 8106 can be configured to allow additional multimodal input, such as an image or a video, of the additional meal and meal visualization model 1306 is configured to detect meal components of the additional multimodal input, in a similar manner as discussed above. Additional meal component elements can be generated to visually represent the detected meal components of the additional multimodal input, and displayed proximate to meal component elements 8102A-E by graphical user interface 8000H.

Recommendation component 8108 is configured to display recommendation text provided in output from meal visualization model 1306. Output from meal visualization model 1306 can include detected meal objects (e.g., corresponding to meal component elements 8102A-E (i.e., ingredients)), glycemic impact values (e.g., corresponding to score indicators 8104A-E), and recommendation text which is text personalized based on the detected meal objects. Recommendation component 8108 is configured to include a user selectable visual element (e.g., a checkbox, a radio button), also referred to herein as a selectable object or selectable option, for selecting a recommendation for updating information in summary component 8112A and/or glucose impact component 8110A. Recommendation component 8108 may be implemented with similar functionality as discussed with respect to recommendation component 8086.

Glucose impact component 8110A is configured to display an overall glycemic impact score associated with the combination of meal components. In some embodiments, this glycemic impact score may be generated based on an algorithm separate from meal visualization model 1306 as discussed further herein, or can be provided as input via a prompt template for execution by meal visualization model 1306. In some embodiments, the glycemic impact score can include low, moderate, and high impacts and visually represented with corresponding colors, such as green for low impact, yellow for moderate impact, and orange/red for high impact.

FIG. 8I depicts an exemplary graphical user interface 8000I configured to display updated glucose impact component 8110B and updated summary component 8112B, which can be generated based on additional user input received, such as via user inputs received in graphical user interface 8000H. In some embodiments, updated glucose impact component 8110B can depict the actual meal score (i.e. actual glycemic impact score) of the meal based on the user's glucose data collected before and after the meal. After being generated, updated glucose impact component 8110B can replace prior visualization elements associated with prior predicted glucose impact scores, such as glucose impact component 8110A. In some embodiments, updated glucose impact component 8110B can also be updated to reflect the impact of concatenated meals, such as meals consumed within a predetermined period from each other, as discussed above.

As with other graphical user interfaces discussed herein, the graphical user interfaces of any of FIG. 8H-8I may be displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces.

FIGS. 9A-9C are diagrams of a graphical user interface associated with the multimodal meal visualization system for correction mechanisms associated with AI-assisted meal logging according to an embodiment. In some embodiments, the input into meal visualization model 1306 for generating output to be used to generate graphical user interfaces of FIGS. 9A-9C can be provided via a prompt generator (e.g. prompt generator 1610, prompt library 1645, or prompt engine 1651) using predefined prompt templates. Output of meal visualization model 1306 may include a structured output file that is fed into a visualization generator (e.g., visualization generator 1312) for generating respective graphical user interfaces based on information provided in the structured output file.

FIGS. 9A-9C are diagrams of a graphical user interface associated with the multimodal meal visualization system for extracting meal information (e.g., metadata) according to an embodiment. In some embodiments, the graphical user interfaces of FIGS. 9A-9C are generated by visualization generator 1312 in response to receiving output from meal visualization model 1306.

FIG. 9A depicts a graphical user interface 9000A comprising meal component or ingredient identifiers 9002A, nutrition text component 9004A (e.g., macro nutrient information), narrative text component 9006A (e.g. narrative insights derived from the meal and based on user health information), and input component 9010 (e.g., indicator to add another entry). Meal component identifiers 9002A may include one or more meal component identifiers with functionality similar to meal component identifiers (e.g., graphical elements 8002B, 8004B, 8006B, and 8008B as depicted in FIG. 8B). Upon selection of input component 9010, graphical user interface 9000A may transition to graphical user interface 9000B as shown in FIG. 9B. Graphical user interface 9000B may include user input component 9008, which is configured to receive additional user textual input. In some embodiments, user input component 9008 may be configured to receive additional multimodal input such as an audio file (e.g., a user provided description of a meal received via a microphone of meal visualization device (e.g., “Breaded tilapia with penne pasta with marinara sauce and veggies”)) and/or additional image data (e.g., an image or picture depicting one or more additional meal components as objects in the image).

FIG. 9C depicts graphical user interface 9000C that is configured to display the updated information in meal component identifiers 9002C, nutrition text component 9004C, and narrative text component 9006C. Meal component identifiers 9002C may be updated to include the meal component identifier(s) detected based on the additional input. Nutrition text component 9004A may be updated to include nutrition information associated with the meal component identifier. More or fewer interface components may be updated based on the nature of the change made.

Importantly, meal visualization model 1306 may be configured to detect a meal component identifier(s) associated with additional input received via user input component 9008 and further configured to update the information in meal component identifiers 9002A, nutrition text component 9004A, and narrative text component 9006A. The additional input received via user input component 9008 may be the same or different type of input that was used to generate information for meal component identifiers 9002A, nutrition text component 9004A, narrative text component 9006A. For example, information for meal component identifiers 9002A, nutrition text component 9004A, narrative text component 9006A may be generated by meal visualization model 1306 based on image data and the additional input provided via input component 9008 based on audio data and/or text data.

Narrative text component 9006A may be configured to display meal textual information generated by meal visualization model 1306 based on the meal classification information (e.g., meal component identifiers). The meal textual information differs from summary text in that the meal textual information is generated text provided by meal visualization model 1306 and formatted into a narrative format (e.g., sentences).

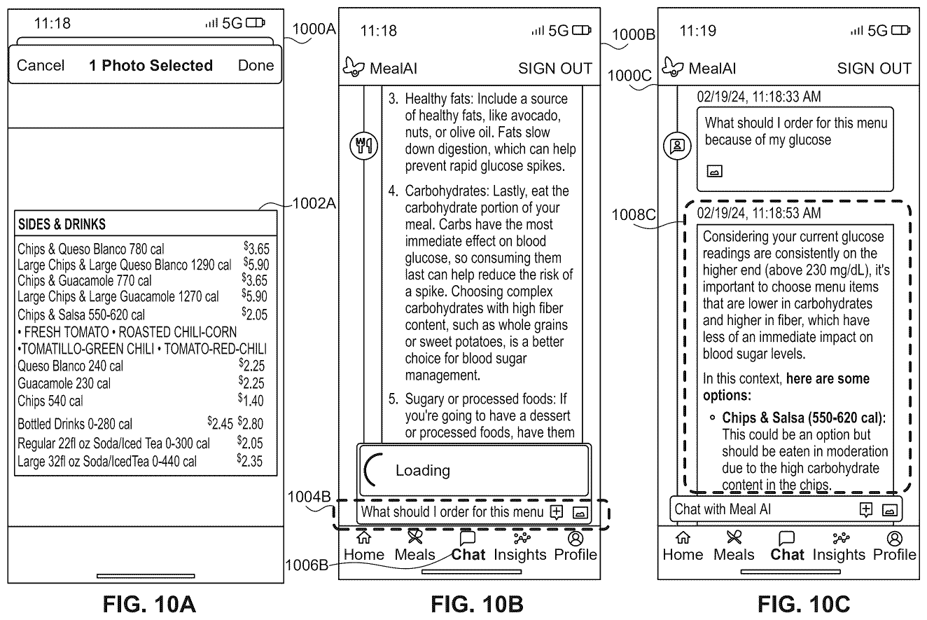

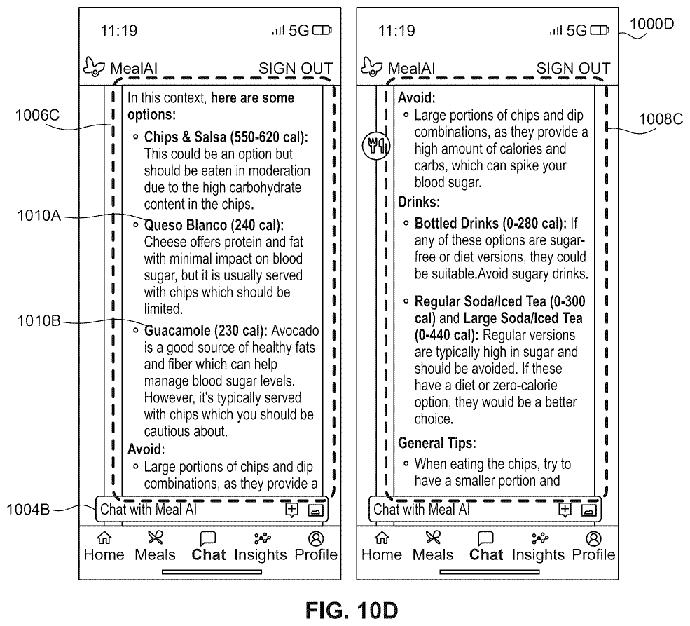

FIGS. 10A-10D are diagrams of a graphical user interface associated with the multimodal meal visualization system for providing AI-assisted chat intent and meal selection according to an embodiment. As with the other graphical user interfaces discussed herein, the graphical user interfaces of FIG. 10A-10D are displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces. In some embodiments, the input into meal visualization model 1306 for generating output to be used to generate graphical user interfaces of FIGS. 10A-10D can be provided via a prompt generator (e.g. prompt generator 1610, prompt library 1645, or prompt engine 1651) using predefined prompt templates. Output of meal visualization model 1306 may include a structured output file that is fed into a visualization generator (e.g., visualization generator 1312) for generating respective graphical user interfaces based on information provided in the structured output file.

FIG. 10A depicts graphical user interface 1000A, which is configured to receive multimodal input, such as image data 1002A (e.g., an image of a menu or a meal). In some embodiments, graphical user interface 1000A may be implemented like graphical user interface 8000A and is configured to receive one or more of text data, audio data, and image data. In some embodiments, graphical user interface 1000A is displayed in response to a user input provided in graphical user interface 1000B, shown in FIG. 10B.

In some embodiments, image data 1002A may comprise an image of a meal (e.g., as discussed with respect to FIG. 8). In other embodiments, image data 1002A may comprise an image of textual data describing meal components, such as, nutrition information or a menu. FIG. 10A depicts an image of a menu with text describing meal components. Meal visualization model 1306 is configured to extract meal information (e.g., metadata) from the image, such as the meal component identifiers/ingredients associated with any detected meal objects in the image. Meal visualization model 1306 may also be configured to access a food database containing nutrition information for a particular restaurant or venue. Meal visualization model 1306 can query this food database with information extracted from the image, input from the user, and/or location information for the user. When location information is used, the user may be asked via the graphical user interface for permission to access location information determined by the meal visualization device 1360 or may be requested to input it by, for example, an additional input component (like input component 8018B and other similar input components). The extracted information (e.g., metadata of the image data) may then be utilized in combination with additional input, as described with respect to graphical user interface 1000B.

Graphical user interface 1000B depicts an icon 1006B and additional user input interface 1004B. Icon 1006B may be implemented with similar functionality described with respect to icons 8020, 8022, and 8024 in FIG. 8B. In some embodiments, icon 1006B may be configured to initiate display of the additional user input interface 1004B and access to an interactive chat functionality of meal visualization model 1306. Additional user input interface 1004B may include one or more input components, such as a text input component and a multimodal input component, where each input component may be separately displayed and accessible via user input (e.g., such as touch input on a touchscreen of meal visualization device).

In some embodiments, additional user input interface 1004B may receive a user query within a text input component and one or more additional inputs via a multimodal input component. For example, selection of a camera input may transfer graphical user interface 1000B to graphical user interface 1000A for receiving image data.

In some embodiments, meal visualization model 1306 may be configured to identify an intent of the user query, and further configured to generate based on a combination of the identified intent and the one or more inputs (e.g., image data), a dynamic meal recommendation by meal visualization model 1306. The dynamic meal recommendation may be displayed within chat interface component 1008C of graphical user interface 1000C, shown in FIG. 10C.

In some embodiments, the one or more inputs used by meal visualization model 1306 to generate the dynamic meal recommendation includes user information such as user medical history, user cohort information, user analyte history, user analyte levels, and user meal history, just to name a few examples. Meal visualization model 1306 may be configured to select one or more of the inputs configured by a remote server and/or within the software application installed on meal visualization device 1306. Remote server may be embodied as one or more of remote application server 155, application storefront 160, trusted computer system 180, or other remote servers accessed via cloud 190. Selection of the one or more inputs may be based on desired personalization settings. For example, meal visualization model 1306 may generate the dynamic meal recommendation tuned to the user's current analyte levels to generate a dynamic meal recommendation that is specific to adjusting the user's current analyte levels. As another example, meal visualization model 1306 may utilize a user's analyte history and/or meal history to generate a dynamic meal recommendation that is tuned to adjust a user's glucose levels over a greater time period (which captures, for example, glucose responses to different meals).

Another example of user information includes user meal preferences such as diabetes diagnosis, diabetes management, dietary preferences, dietary restrictions, and other conditions. Selectable options of diabetes diagnosis can include type 2 diabetes, type 1 diabetes, and pre-diabetes. Selectable options of diabetes management includes diet, exercise, insulin pump, glucagon-like peptide (GLP)-1, long-acting or basal insulin, non-insulin injectables, oral medications (tablets or pills), short or rapid acting insulin, and bolus insulin. One or more of these selectable options may be selected as being applicable to the user. Selectable options of dietary preferences include all food, vegetarian, vegan, pescatarian, keto, paleo, gluten-free, and dairy-free. Selectable options of dietary restrictions include any allergies including peanuts, tree nuts, milk, eggs, fish, shellfish, soy, wheat, and mushrooms. One or more of these selectable options may be selected as being applicable to the user. Selectable options of other conditions can include mobility assistance required, cognitive considerations, sensory sensitivities, pregnancy or nursing, traveling or on vacation, shift work or irregular schedule, athletic training, stress management needs, limited kitchen access limited time for meal preparation, cultural or religious dietary practices, and recovering from illness or surgery. One or more of these selectable options may be selected as being applicable to the user. Various mechanisms may be used for selecting the various selectable options as being applicable to the user. For example, the user may update their information on a graphical user interface to configure their user profile. The user profile may be accessed by a suitable icon, for example the “Profile” icon as displayed in FIGS. 8B-8E, FIG. 8H, FIG. 8I, FIG. 9A, FIG. 9C, FIGS. 10B-10D, FIGS. 11B-11C, and FIGS. 12A-12B. Alternatively, user information may be set via a remote server based on information already known about the user (e.g. diabetes diagnosis, diabetes management). Remote server may be embodied as one or more of remote application server 155, application storefront 160, trusted computer system 180, or other remote servers accessed via cloud 190.

In some aspects, these selectable options along with other inputs (e.g., image, text, user data) may be combined by a prompt generator (e.g., prompt generator 1610, prompt library 1645, or prompt engine 1651) to generate a prompt for input to meal visualization model 1306. A predefined prompt template may include specific placeholders associated with text within the prompt template for assigning specific weights and instructions to each of the inputs within the prompt. For example, the predefined prompt template may include placeholders for user meal preferences, as discussed above, and include instructions associated with these placeholders how these preferences should be utilized by meal visualization model 1306 to generate the output to visualization generator 1312. For example, user meal preference may impact how the meal visualization model 1306 determines meal classification information, including determining a meal identifier of a meal object. In another example, user meal preference may impact the recommendations determined by visualization model 1306. For example, a vegetarian user will not be provided with recommendations that involve meat. As another example, user meal preference may be utilized to determine candidate meal times (e.g., when meal-start times have not been provided with meal inputs, when meal information for multiple meals are uploaded at one time, when meal information for a meal is not synchronized with the actual meal start time). For example, a user meal preference may indicate that pizza as a preferred breakfast so meal information associated with pizza may be associated with breakfast meals.

For example, the predefined prompt template may include instructions to assign higher weights to vegetarian options more if a user meal preference indicates that the user is vegan or vegetarian. Accordingly, meal visualization model 1306 may assign higher probabilities to vegetarian options when performing meal detection of received input (e.g., favoring tofu versus chicken for detected meal components that are white).

FIG. 10D depicts graphical user interface 1000D comprising another embodiment of chat interface component 1008C which displays the dynamic meal recommendation provided by meal visualization model 1306. The recommendation in FIG. 10D includes a nutrition text component (e.g. calories), meal component identifiers, and a narrative text component for each recommendation. Additional recommendations on meal components to avoid and/or general tips are provided. In some embodiments, graphical user interface 1000D may receive user input for selecting one or more of the meal components, such as meal component 1010A and meal component 1010B, identified within the displayed dynamic meal recommendation. User input may be via selecting the one or more meal components (e.g., a touch-based input) or via user input interface 1004B. In any event, as mentioned elsewhere herein, meal visualization device 1310 and/or user device 1302 may incorporate a display to display the graphical user interfaces, and the display may be a touch screen so that the user can interact with interface components of the graphical user interfaces, in particular by touch-based input. Graphical user interface 1000D may provide the selected meal components to meal visualization model 1306 for additional processing, such as updating the dynamic meal recommendation or generating graphical user interfaces 9000A-C and their various components (e.g., meal component identifiers 9002A, nutrition text component 9004A, and narrative text component 9006A).

In some aspects, chat interface component 1008C is configured to receive real-time text queries (e.g., queries/chat data 1606 discussed in FIG. 16A). Chat interface component 1008C may provide an interface for receiving real-time text queries and providing the queries as input to meal visualization model 1306 for processing the text query, identifying intent of the query, identifying needed sources of data for responding to the query and the intent, and generating responses for display on chat interface component 1008C.

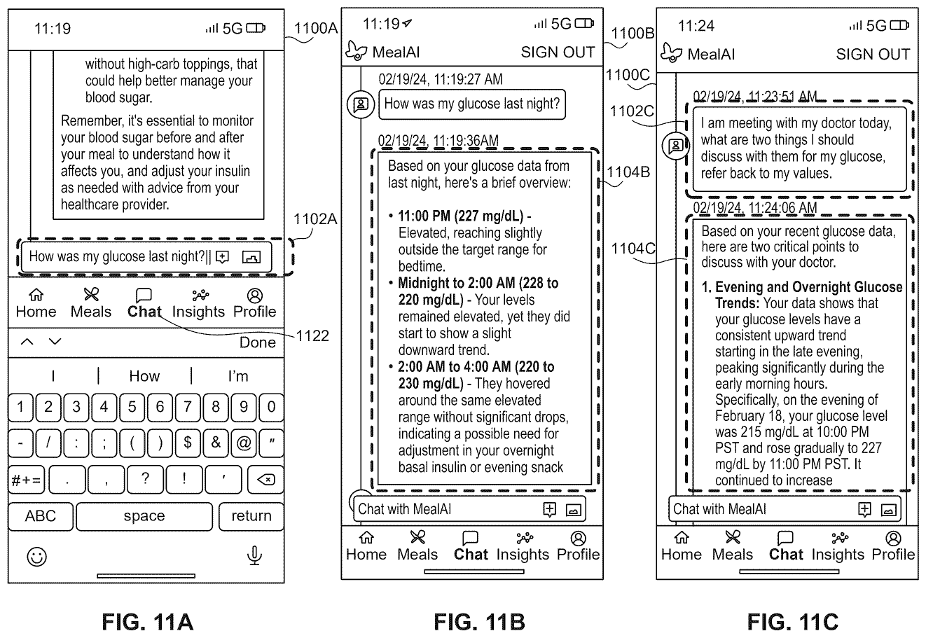

FIGS. 11A-11C are diagrams of a graphical user interface associated with the multimodal meal visualization system for providing AI-assisted health analysis according to an embodiment. In various embodiments, the graphical user interfaces of FIG. 11A-11C are displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces. In some embodiments, the input into meal visualization model 1306 for generating output to be used to generate graphical user interfaces of FIGS. 11A-11C can be provided via a prompt generator (e.g. prompt generator 1610, prompt library 1645, or prompt engine 1651) using predefined prompt templates. Output of meal visualization model 1306 may include a structured output file that is fed into a visualization generator (e.g., visualization generator 1312) for generating respective graphical user interfaces based on information provided in the structured output file.

FIG. 11A depicts graphical user interface 1100A, which may be initiated via selection of icon 1122 (e.g., similar to icon 8022), which may be configured to access an interactive chat function of meal visualization model 1306. Graphical user interface 1100A comprises user input interface 1102A for receiving user queries. Meal visualization model 1306 is configured to identify an intent of received user queries, and in some embodiments, identifying additional relevant inputs to be included as inputs with the identified intent for generating a response to user queries.

For example, meal visualization model 1306 may determine an intent for a user query “How was my glucose last night” as requiring generating text related to the user's glucose levels for a specific time frame (e.g., 8 pm to 6 am) or during an activity (e.g., while the user was sleeping as entered by the user or automatically collected using a sleep detector, such as a commercial smart watch or heart rate monitor configured to determine sleeping patters based on cardia signals). Based on this determined intent, meal visualization model 1306 may further determine the necessary additional inputs that are required to generate a relevant response. For example, based on identifying glucose level and a time period, meal visualization model 1306 may retrieve relevant inputs from user analyte levels and user analyte history, and using the determined intent, meal visualization model 1306 may be configured to generate text responsive to the inputs and intent.

As another example, meal visualization model 1306 may determine an intent for a user query “What should I order from this menu based on my glucose” as requiring generating text related to provided additional input (e.g., text data, image data) in relation to the user's glucose levels. Based on this determined intent of the query data, meal visualization model 1306 may further determine the necessary additional inputs that are required to generate a relevant response. For example, based on identifying the needed additional input and user's current glucose levels, meal visualization model 1306 may retrieve relevant inputs from user historical analyte levels, user meal history, user current analyte level (e.g., including glucose rate of change), medication information (e.g., insulin on board, the time and amount of the last insulin injection, the time and amount of a planned insulin injection), and perform relevant object recognition on the additional input to identify the meal objects within the provided input. Based on the determined inputs and the determined intent, meal visualization model 1306 may be configured to generate text responsive to the inputs and intent (e.g., recommendations).

FIG. 11B depicts graphical user interface 1100B comprising chat interface 1104B, which may be configured to display generated text from meal visualization model 1306.

FIG. 11C depicts graphical user interface 1100C comprising chat interface 1104C, which may be configured to display user inputs, provided via user interface 1102A, and generated text from meal visualization model 1306. In this embodiment, follow-up user queries may be received via user interface 1102C that require meal visualization model 1306 to remember previous queries, determine the intent of the follow-up user query, and repeat the process of determining the needed additional inputs (e.g., user analyte history, user analyte levels, user meal history) to generate text to respond to the follow-up user query. The additional generated text may then be displayed in chat interface 1104C.

FIGS. 12A-12B are diagrams of a graphical user interface associated with the multimodal meal visualization system for presenting AI-assisted health insights according to an embodiment. In various embodiments, the graphical user interfaces of FIG. 12A-12B are displayed on the meal visualization device 1310, which may be the same device as user device 1302 in some implementations. Meal visualization device 1310 and/or user device 1302 may incorporate a display to display the graphical user interfaces. In some instances, the display may be a touch screen so that the user can interact with interface components of the graphical user interfaces. In some embodiments, the input into meal visualization model 1306 for generating output to be used to generate graphical user interfaces of FIGS. 12A-12B can be provided via a prompt generator (e.g. prompt generator 1610, prompt library 1645, or prompt engine 1651) using predefined prompt templates. Output of meal visualization model 1306 may include a structured output file that is fed into a visualization generator (e.g., visualization generator 1312) for generating respective graphical user interfaces based on information provided in the structured output file.

FIG. 12A depicts graphical user interface 1202A comprising insights component 1202 and icon 1204. Selection of icon 1204 is configured to trigger interaction with insights functionality of meal visualization model 1306. Features of insights functionality may be pre-configured in meal visualization model 1306, such as providing predefined categories of insight categories responsive to the selection of icon 1204. The insights may be glycemic insights, i.e. insights related to the user's analyte levels. Examples of predefined categories include providing statistical analysis of a user's analyte information for a specified time period (e.g., overnight, past 2 days, past week), analyte events (e.g., time in range, hyperglycemic (high) events, hypoglycemic (low) events), and summary information. In particular, insights component 1202 of FIG. 12A shows average glucose, standard deviation of glucose, time in range (i.e., not hypoglycemic or hyperglycemic), the number of hyperglycemic events and the number of hypoglycemic events. Any combination of these may be included in insights component 1202. Text generated by meal visualization model 1306 related to the insights functionality may be displayed within an interface component 1202 of graphical user interface 1202A.

In some embodiments, graphical user interface 1202A may be configured to include a user input icon 1206 configured to trigger interaction with chat functionality with meal visualization model 1306. Queries or multimodal inputs received upon selection of user input icon 1206 may be used by meal visualization model 1306 to further update information displayed within interface component 1202.

FIG. 12B depicts graphical user interface 1200B comprising additional insights component 1208 that may be generated independently by meal visualization model 1306 or in response to additional input (e.g., additional user query, additional multimodal input such as image data, text data, and/or audio data).

Example of additional insights component 1208 include narrative information that is generated by meal visualization model 1306 that can include user analysis such as analysis of a user's average glucose, a summary of the user's glucose history, recommended actions for user meals, a summary of user glucose metrics such as standard deviation from user's glucose history, and other narrative information provided in insights component 1202.

FIG. 13 is a block diagram of meal visualization system 1300 according to an embodiment. Meal visualization system 1300 comprises various input sources such as user input from user device 1302, such as user multimodal input 1302A and user query 1302B, and additional input sources 1304, such as user data 1304A, continuous analyte data 1304B, additional multimodal meal data 1304C, and backend system data 1304D. These particular input sources are merely an example, and other input sources may be present instead or in addition, as discussed further herein. Meal visualization system 1300 may further include meal visualization model 1306, meal visualization device 1310, and insulin delivery device 1314. It should be appreciated that meal visualization system 1300 is merely an example embodiment.

In some embodiments, analyte monitoring system 100 of FIG. 1A and/or analyte monitoring system 100a of FIG. 1B may form part of meal visualization system 1300. In other embodiments, meal visualization system 1300 may form part of analyte monitoring system 100 of FIG. 1A and/or analyte monitoring system 100a of FIG. 1B. In further embodiments, the meal visualization system 1300 may be used in conjunction with the analyte monitoring system 100 of FIG. 1A and/or analyte monitoring system 100a of FIG. 1B.

As noted above, the meal visualization model 1306 is a trained model. In some embodiments, a pre-existing/pre-trained machine learning model may be used for the meal visualization model. In other embodiments, when not using a pre-existing/pre-trained machine learning model, meal visualization model 1306 may include one or more sub-models each configured to perform a different function of the functions of meal visualization model 1306 described herein. One or more of the sub-models involve artificial intelligence (AI), and as such may be referred to as a trained sub-model of meal visualization model 1306. More generally, because the meal visualization model 1306 includes at least one sub-model that is trained, the meal visualization model may also be referred to herein as a trained meal visualization model, even if not all sub-models of the meal visualization model 1306 are trained or otherwise AI-assisted.

Meal visualization model 1306 may be implemented in one or more devices of meal visualization system 1300. For example, meal visualization model 1306 may be implemented as part of a software application installed in meal visualization device 1310. The software application may be installed from application storefront server 160. Examples of a meal visualization device 1310 includes a user device (e.g., user device 1302, data receiving device 120, multi-purpose data receiving device 130, user device 140, or local computer 170) such as a smart phone, tablet, or a sensor reader device that is configured to communicate with an analyst sensor (e.g., analyte sensor 104 or sensor control device 102). As another example, meal visualization model 1306 may be implemented remote from meal visualization device 1310, such as a backend system (i.e. the system from which backend system data 1304D is received). For example, meal visualization device 1310 may be trusted computer system 180 or a different server connected to cloud 190 such as remote application server 155. As another example, meal visualization model 1306 may be implemented in a distributed manner between multiple devices (e.g., meal visualization device 1310 and backend system). In this example, functions of meal visualization model 1306 may be split between multiple “lightweight” visualization models, which are configured to communicate with each other when performing the meal visualization analysis.

Meal visualization model 1306 may receive inputs as part of the meal visualization analysis. In general, the meal visualization model 1306 receives image data and additional user input. The additional user input may include user input provided via the user device 1302, including user multimodal input 1302A and user query 1302B. Additionally or alternatively, the additional user input may contain any information related to the user, for example user data 1304A, continuous analyte data 1304B, and the like. In some embodiments, the meal visualization model 1306 may receive the image data and/or additional user data from user device 1302. In such embodiments, the meal visualization device 1310 may be the same device as the user device 1302. In other words, user device 1302 may be any one of data receiving device 120, multi-purpose data receiving device 130, user device 140, or local computer 170 as discussed elsewhere herein. Other sources of image data and additional user data are discussed herein

In some embodiments, in addition to receiving image data, meal visualization model 1306 may receive additional user input from user device 1302 (e.g., provided as user multimodal input 1302A and/or user query 1302B via meal visualization device 1306) and, in some embodiments, may be trained to identify an intent associated with the user input. User input may take a variety of forms including any combination of a text query (including an initial query and follow-up queries), additional image data, video data, audio data, and textual data. Textual data may be unstructured or structured and received from a variety of different sources include analyte sensors, analyte databases, user profiles, and electronic health record (her) systems. In some embodiments, user input may include a user query and may be received in real-time via a chat interface provided by visualization generator 1312. The chat interface can be configured to receive unstructured text queries and provides the unstructured text queries as additional input to meal visualization model 1306. Other additional user input is discussed with respect to FIGS. 8A-12B. For example, additional user input may take the form of one or more of: user medical history (including electronic medical record data), user meal history, textual data, audio data, video data, or additional image data; meal information associated with image data; query data; analyte data; wearable device data; user feedback or correction; user information; and combinations thereof. Each of these is discussed in further detail above.

In some instances, the additional user input may be received in real time. In other words, the meal visualization model 1306 uses the additional user input immediately when it is input by the user. In other instances, the additional user input may be received in advance of it being used by the meal visualization model 1306. For example, user information (e.g. meal preferences such as diabetes diagnosis, diabetes management, dietary preferences, dietary restrictions, and other conditions) may have been input by the user into user device 1302 when the software application is first downloaded to user device 1302. In such embodiments, receiving additional user input may comprise storing the additional user input in memory (e.g. memory of user device 1302) and later retrieving the additional user input from the memory so it can be used by the meal visualization model 1306.

In some embodiments, receiving the additional user input may involve obtaining data from another device, such as a continuous analyte monitor (e.g. analyte sensor 104, sensor control device 102). The user may then provide input on the obtained data using the user device 1302. In this way, the additional user input may relate to or comprise data from another device, such as a user device (other than user device 1302), a remote server, a continuous analyte monitor, an insulin delivery device, one or more sensors, one or more external databases, and the like. For example, analyte data may be provided to the user device 1302 from the continuous analyte monitor. The user may be requested by the user device 1302 to provide the analyte data to the meal visualization model 1306, and the user response becomes additional user input containing the analyte data

User multimodal input 1302A includes image data such as images taken by a camera of a user device 1302 (e.g., meal visualization device 1310) and which may be provided either from an image library or via an image and/or video capture feature of the user device 1302. FIG. 8A and FIG. 8G provide further details about the process of providing image data. In embodiments where the user multimodal input 1302A includes image data such as images taken by the camera of user device 1302 (e.g. meal visualization device 1310), such devices may be referred to as an imaging device. In general, the imaging device has a camera integrated into the imaging device. Such a camera may also be considered integrated into the meal visualization system 1300. User multimodal input 1302A may also include audio data such as recordings taken by a microphone of a user device 1302. Other types of multimodal input 1302A may also be used by the meal visualization model 1306, as discussed elsewhere herein.

In embodiments, user multimodal input 1302A further can include feedback input provided in response to providing a predicted glucose impact. Meal visualization model 1306 is configured to determine whether a difference between the predicted glucose impact (e.g., a predicted glucose trend) and the actual glucose impact meets a threshold amount. If the difference exceeds the threshold amount, meal visualization mode 1306 is configured to perform a retrospective analysis of the predicted glucose impact, which can include, generating visualizations for receiving user feedback regarding parameters used for generating the predicted glucose impact including the detected meal components, the meal time, and other user behavior. In embodiments, meal visualization model 1306 is configured to generate an audit trail of the end-to-end process for generating the predicted glucose impact, including the detected meal components, glucose values, any data provided from user device 1302, any data provided from additional data sources 1304, prompts, output, and calculations involved for generating the meal impact score (e.g., the adjusted delta to peak, a decision-region plot, and recommendations selected and not selected by the user—discussed further herein). Meal visualization model 1306 can be configured to perform a retrospective analysis of the audit trail to identify reasons for the threshold difference between a predicted glucose impact and actual glucose impact. In embodiments, meal visualization model 1306 can use the actual glucose impact and the audit trail as inputs for the retrospective analysis to identify which steps or inputs most likely resulted in the difference between the predicted glucose impact and the actual impact.

User query 1302B, also referred to herein as query data, includes textual data (e.g., text input) provided via an input interface of a user device 1302 and includes, in some embodiments, an intent to be detected by meal visualization model 1306. This is discussed in further detail with respect to FIGS. 10A-10C. In some instances, user query 1302B may include an initial query and follow up queries.

Meal visualization model 1306 is trained, in some embodiments, to identify additional data sources 1304 needed for performing the meal visualization analysis based on the user input from user device 1302. In other embodiments, the additional data sources 1304 may be referred to in the prompt template (i.e. the prompt template is customized to reference certain additional data sources and/or additional data therefrom). In other embodiments, the data from the additional data sources 1304 (for example, additional user data or additional data (discussed below)) may be included in the prompt template. Additional input sources 1304 includes, for example, user data 1304A, continuous analyte data 1304B (which is also a type of user data), additional multimodal meal data 1304C, and backend system data 1304D (which may also contain user data). Other input sources may be used. Additional multimodal meal data 1304C may include, for example, any combination of image data, audio data, and text data identifying one or more meals or meals components. These input sources may be from various physical devices or systems, as further discussed below.

In some embodiments, in addition to the image data and the additional user input, the meal visualization model 1306 may require input of additional data (i.e., not user data). For example, one or more of the following additional data sources other than those shown in FIG. 13 may be used: a food database; a nutrition database; a food composition database (providing nutritional profiles of foods); a recipe database; a food images database; anonymized healthcare databases; and/or electronic health records. The nature of the additional data from each of these sources is discussed with respect to FIGS. 8A-12B. In some embodiments, these additional data sources may be in the prompt template (i.e. the prompt template is customized to reference certain additional data sources). In other embodiments, the data from these additional data sources (for example, additional user data or additional data) may be included in the prompt template.

As one example, based on a detected intent within a user query, meal visualization model 1306 is trained to identify any additional data needed to perform the meal visualization analysis responsive to the detected intent. Meal visualization model 1306 may then be further configured to retrieve information from one or more of the additional input sources 1304.

As discussed, image data is generally obtained from the user device 1302. Additional user data may be received from the user device 1302 and/or additional data sources, including one or more of the additional data sources 1304 shown in FIG. 13 or other additional data sources. Additional (non-user) data is generally received from the additional data sources and not the user. In some embodiments, user input 1302A from user device 1302 may be implemented as input based on user action, such as user queries via a chat interface or uploading image, video, or audio data via a user device (e.g., user device 1302) while additional input sources 1304 may be implemented as repositories of data that is retrieved by meal visualization model 1306 to be used as input for the meal visualization analysis. In other embodiments, the user device 1302 may retrieve the data and provide it in a prompt template to the meal visualization model 1306.

User data 1304A, i.e., user data not from the user device 1302, may include data related to the user such as profile information (e.g., settings), user EHR data, user medical history information, and user meal history information. Additionally or alternatively, user data 1304A may include any of the other user information discussed herein. Such user information may include meal preferences (e.g. diabetes diagnosis, diabetes management, dietary preferences, dietary restrictions, and other conditions). Other user data 1304A may include age, weight, culture and religion, and the like. In embodiments where the user device 1302 is one of data receiving device 120, multi-purpose data receiving device 130, user device 140, or local computer 170 as discussed elsewhere herein, user data 1304A may come from any other of these devices. Remote servers such as remote application server 155, application storefront 160, trusted computer system 180, or other remote servers accessed via cloud 190, may also store user data 1304A.

Continuous analyte data 1304B may include user analyte data, which may be provided via an analyte sensor (e.g., analyte sensor 104 or sensor control device 102), also referred to herein as a continuous analyte monitor, in communication with meal visualization model 1306, and analyte history data, which be retrieved from local memory (e.g., on the user device 1302) or remotely (e.g., part of backend system data 1304D). In particular, when the continuous analyte monitor is embodied as analyte sensor 104, the analyte data may be retrieved from memory 163. When user device 1302 is embodied as data receiving device 120, the analyte history data may be retrieved from memory 223, memory 225 and/or memory 230, or from a remote server such as remote application server 155, application storefront 160, trusted computer system 180, or other remote servers accessed via cloud 190. The process of collecting analyte data and devices required for this process are discussed in the analyte monitoring section above. Any feature described in the analyze monitoring section may be incorporated in the systems, devices and methods of the disclosure for multimodal meal visualization and glycemic impact analysis discuss at FIG. 8A onwards.

Additional multimodal meal data 1304C may include any multimodal data received separate from user input from user device 1302, such as meal data that can be retrieved from user social accounts, user journal entries, websites (e.g., restaurant websites), food databases, and other user devices that have provided information related to the current meal and/or restaurant.

For example, in some embodiments, meal visualization model 1306 may determine additional inputs are needed to respond to a user query involving a particular meal, a particular meal component, and/or a particular location (e.g., a restaurant). Meal visualization model 1306 may be configured to retrieve related information associated with the particular meal, particular meal component and/or the particular location that was provided by other user devices. One example includes another user device providing image data of a meal at a particular restaurant, which meal visualization model 1306 may determine may be needed respond to a query that involves the same meal and/or the same restaurant. Backend system data 1304D may be from one or more backend system that provides additional data related to the user such as an EHR system, a meal database, a population database, a health care provider (HCP) database, just to name a few examples. Other examples of backend systems are given throughout the specification. Remote servers such as remote application server 155, application storefront 160, trusted computer system 180, or other remote servers accessed via cloud 190, are examples of backend systems.

Meal visualization model 1306 can receive additional inputs (e.g., query text) via a chat interface that is provided by visualization generator 1312 for receiving user queries from user devices such as meal visualization device 1310. Other inputs can be provided to meal visualization model 1306 via the chat interface such as user feedback and/or user corrections.

Meal visualization device 1310 comprises visualization generator 1312 that is in communication with meal visualization model 1306 and is configured to generate user interfaces, particularly graphical user interfaces, based on outputs provided by meal visualization model 1306. In some embodiments, meal visualization device 1310 may be implemented as an exemplary user device 1302, where user inputs are received via interaction with one or more visual interface components provided by visualization generator 1312. Examples of visual interface components are discussed with respect to FIGS. 8A-8I, 9A-9C, 10A-10D, 11A-11C, 12A, and 12B.

Meal visualization model 1306 may receive a combination of any of inputs provided by user device 1302 and additional data sources 1304 from a prompt generator (e.g., prompt generator 1610, prompt library 1645, or prompt engine 1651) which formats the inputs into a predefined prompt template that can include placeholders into which inputs may be inserted. Further detail about prompt generators is discussed with respect to FIG. 16A and FIG. 16B.

In some embodiments, meal visualization model 1306 may be configured to connect with insulin delivery device 1314, which is capable of injecting or infusing insulin into the body of an individual wearing an analyte sensor (e.g., analyte sensor 104 or sensor control device 102). Insulin delivery device 1314 can include an insulin pen, a smart insulin pen, smart insulin pen caps, a drug reservoir, a pump, an infusion tube, and/or an infusion cannula configured for at least partial implantation into the user's body. The pump can deliver insulin from the reservoir, through the tube, and then through the cannula into the user's body. Insulin delivery device 1314 can include instructions, executable by the processor, to control the pump and the amount of insulin delivered. These instructions can also cause calculation of insulin delivery amounts and durations (e.g., a bolus infusion and/or a basal infusion profile) based on analyte level measurements obtained directly or indirectly from an analyte sensor (e.g., analyte sensor 104 or sensor control device 102). Alternatively, calculations of insulin delivery amounts and durations, and the control of the pump, can be performed by meal visualization model 1306 and instructions based on these calculations may be transmitted to insulin delivery device 1314 for delivering the specified insulin amount. Insulin delivery device 1314 can be configured to communicate directly with meal visualization model 1306 in the form of a closed loop or semi-closed loop system.

In some embodiments, meal visualization model 1306 may be configured to generate structured output files that, in some embodiments, include meal-specific insulin dose recommendations (e.g., included as part of glycemic impact prediction 1624) to address the start-up problem found in conventional dose recommendation systems. In some embodiments, the meal-specific insulin dose recommendation may be determined by meal visualization model 1306. The type of meal-specific insulin dose recommendations can be defined by the prompt generated by a prompt generator (e.g. prompt generator 1610, prompt library 1645, or prompt engine 1651) as input to meal visualization model 1306. To avoid this start-up problem, the meal visualization model 1306 can, in some embodiments, rely on population (or cohort) meal data to generate initial recommendations for a user. As the meal visualization model 1306 gathers user specific meal data, the meal visualization model 1306 can use a combination population and user specific meal info, or switch to user-specific meal data only. In some embodiments, the glycemic impact prediction 1624 may be processed or further processed within an insulin dosing algorithm alone or in combination with any user medical data such as current glucose level, insulin on board, last dose information, and glucose rate of range. The insulin dosing algorithm may be specified in the prompt by prompt generator 1610 (or prompt library 1645, prompt engine 1651) or the prompt may include instructions causing meal visualization model 1306 to access an external source for the algorithm. In some embodiments, the algorithm may be stored on a user device (e.g. meal visualization device 1310, user device 1302, data receiving device 120, multi-purpose data receiving device 130, user device 140, or local computer 170), on a remote server or backend system (e.g. remote application server 155, application storefront 160, trusted computer system 180, or other remote servers accessed via cloud 190), or on an insulin delivery device (e.g. insulin delivery device 1314).

In some embodiments, the meal-specific insulin dose recommendations can be based on user data, such as current glucose levels, current glucose rate of change, medication information such as insulin on board, meal information (e.g., meal components, nutrition information associated with the meal components), and predicted glycemic impact scores of user. Other user data, including any of the additional user input discussed herein, may be used in addition or alternatively.

The meal visualization model 1306 may rely on meal data from a population of users to provide dose recommendations to the user. The meal visualization system 1300 may include a database that includes information regarding the amount of insulin taken by users who consume a particular or similar meal and the glucose response following the meal and insulin dose. However, users differ in many respects, for example, two users may have different glucose responses to the same meals, and their glucose levels may respond differently to the same dose of insulin. Accordingly, it is important to determine which users in the population are comparable. Further, it can be difficult to determine which meals taken by others in the population are sufficiently similar. Accordingly, there is a need for a system that can provide recommendations (i.e., insulin dose recommendations) based on population data and that can accurately match the user to similar users in a population and that can accurately match the user's meal to similar meals taken by others in the population. The meal visualization system 1300 disclosed herein relies on artificial intelligence and machine learning models to match the user to similar users and to match the user's meal to similar meals taken by other users.

The meal visualization model 1306 can be trained based on glucose and meal data organized based on a population of users. Glucose data may include historical user glucose data indexed based on meals and meal data may take the form of any multimodal input described above, such as descriptive text, photos or an itemization table of the meal components (or nutritional components) and their amounts in weight or volume.

Meal visualization model 1306 may have access to a database storing information relating to meals consumed by users, the glucose response to the meal, and the insulin dose taken by the user. The meal visualization system 1300 may determine if the insulin dose taken for the meal resulted in glucose levels within a target glucose range in a period of time following the meal and insulin dose (i.e., a post-prandial period). The meal visualization system 1300 may determine and store an indication of whether the insulin dose taken for the meal resulted in glucose levels inside or outside of the target glucose range in the period of time following the meal and insulin dose.

As part of providing meal-specific recommendations, meal visualization model 1306 is configured to identify users from the population data similar to the current user (i.e., the user receiving the recommendation) based on one or more matching characteristics (e.g., gender, age, ethnicity, body weight, medical conditions, meal history, type or degree of diabetes, glucose variations or patterns). Meal visualization model 1306 may further be configured to identify meal data associated with a group of users from the population data with the meal of the current user based on one or more matching characteristics (e.g., meal type, meal components, nutrition information (carbohydrates, protein, fats), time of day of meal, score) and match a particular meal to be consumed by a particular patient to a meal consumed by the population.

Meal visualization model 1306 may match the user to a cohort of users in the population. The matching may be based on one or more user characteristics, (e.g., gender, age, ethnicity, body weight, medical conditions, meal history, type or degree of diabetes, glucose variations or patterns). Meal visualization model 1306 may be used to match the user to a particular cohort of users in the population. Once the user is sorted into a cohort, the meal visualization system 1300 may rely on the meal records for that cohort of users in determining medication recommendations for the user. The sorting or matching of the user with a cohort may be performed ahead of time, in advance of the dose recommendation, such as during initial set-up of the meal visualization system 1300. This may help to expedite processing and matching of meals at the time the dose recommendation is requested. Further, the user matching may occur locally, such as at the receiver device, and the meal matching may occur remotely, such as on a remote server or cloud, or vice versa. Remote server may be embodied as one or more of remote application server 155, application storefront 160, trusted computer system 180, or other remote servers accessed via cloud 190. In alternate embodiments, meal visualization model 1306 may match the user to similar users and may match the meal to similar meals at the same time, i.e., at the time the user requests the dose recommendation.

In operation, meal visualization model 1306 may receive meal information about a meal being consumed, or about to be consumed (e.g., based on one or more multimodal inputs received from a user device), and meal visualization model 1306 may determine the meal components as described herein. Meal visualization model 1306 may identify similar meals consumed by other users in the cohort. Meal visualization model 1306 may then determine a dose recommendation for the user based on the meal records for the cohort. The meal visualization system 1300 may assess if an insulin dose taken by another user in the cohort for the same or similar meal resulted in glucose control. Meal visualization model 1306 may use a machine learning model to determine a dose recommendation based on the insulin doses and glucose responses to the meal by other users in the cohort. Meal visualization model 1306 may only consider insulin doses that resulted in glucose control following the meal, and may not use insulin doses that did not result in glucose control following the meal when providing a dose recommendation. Alternatively, meal visualization model 1306 may account for insulin doses that did not result in glucose control when determining the dose recommendation. For example, if a user in the population administered 5 units of insulin in response to a meal, and the user's glucose levels remained high following the insulin dose and meal, the meal visualization system 1300 may determine that a larger insulin dose was needed for that meal, such as 6 units or more.

Meal visualization model 1306 may output a dose recommendation that is a single dose amount. The dose amount may be based on a plurality of dose amounts taken by users of the cohort in response to the meal, such as an average, weighted average, median dose, among others. In some embodiments, the dose recommendation may include a range of dose amounts. For example, the dose recommendation may inform the user that other users in the cohort having a similar meal administered a dose of 5 units to 8 units. The user can then decide the appropriate dose from the range. In some embodiments, meal visualization model 1306 may provide recommendations for an appropriate dose based on previous dose history, meal history, and other user data accessible to the model. The dose recommendation may include information about insulin doses taken by other users that resulted in glucose levels in range or out of range. For example, the user may be informed that other users who administered 4 units had high glucose following the meal, users who administered 5 to 8 units had glucose in range following the meal, and users who had 9 or more units had glucose below range following the meal.

The meal visualization system 1300 implementing meal visualization model 1306 may titrate the insulin dose for a particular meal based on the user's glucose response following the meal. For example, the first time that the user eats a particular meal, the dose recommendation may be based on population data. The user takes the recommended dose based on the population data and consumes the meal. The user's glucose level may be outside of a target range in the time period following the meal, e.g., 2-3 hours following the meal. The meal visualization system 1300 may store a new meal record including the dose amount, glucose response (e.g., an indication that the glucose level is out of range), and meal information. The next time the user consumes the same meal, the meal visualization system 1300 may adjust the dose amount to drive the glucose levels toward the target glucose range. The amount of adjustment may be proportional to the difference between the glucose level following the meal and the target glucose range. Thus, the population data may be used to determine an initial dose for a particular meal, and the dose amount can be titrated based on user-specific data thereafter.

In some embodiments, the dose recommendation generated by meal visualization model may be based on inputs specified by a prompt template from prompt generator 1610 (or prompt library 1645, or prompt engine 1651). These inputs may include any combination of meal, insulin and glucose data that are specific to the user and/or that are collected from users of insulin delivery devices (AID) devices, such as infusion pumps. Organizing meal, insulin and glucose data based on cohort or populations of users can enhance the amount of data and meal records in the database and to improve accuracy of dose recommendations of users within the same cohort. Dose recommendations may also be stored in databases that may also include information on the total amount of insulin administered to the user by the AID device in a period of time following a particular meal. For example, the period of time may be 1 hour to 6 hours, or 2 hours to 4 hours, or about 3 hours. The total amount of insulin delivered may include a meal bolus in addition to any subsequently delivered insulin in a period of time following the meal, including for example correction doses or basal insulin. For example, if the AID device administers a 6 unit bolus of insulin for the meal, and then delivers a 2 unit correction dose within 3 hours of the meal, the meal visualization system 1300 may record that the user administered 8 units for the meal. The AID device meal records may be used instead of or in combination with the meal records for users who administered insulin using manual injection devices, such as injection pens, or the like.

In some embodiments, meal visualization model 1306 may rely on population data to provide dose recommendations for an initial period of time, such as when the meal visualization system 1300 is first used by the user. For example, a prompt generator, such as prompt generator 1610 may be configured to connect to population or cohort databases and retrieve population data based on the predefined template and other user information. For example, a user may set a preferred cohort or set a privacy setting to not be associated with a particular cohort. This user information may trigger the prompt generator to retrieve or not retrieve relevant cohort data to be included in the predefined prompt template as input to meal visualization model 1306. Other prompt generators such as prompt library 1645 and/or prompt engine 1651 may be used.

The initial period of time may be, for example in a range of 3 days to 14 days, and may be 3 or more days, 5 or more days, 7 or more days, 10 or more days, 14 or more days, among other periods of time. Meal visualization model 1306 can collect user-specific meal data during the initial period. Once user-specific meal data has been collected, the meal visualization system 1300 may begin providing recommendations based at least in part on the user-specific meal data. Dose recommendations may be based on a combination of population data and available user-specific meal data. The user-specific meal data may be weighted more heavily that the population data. Meal visualization model 1306 may continue to provide dose recommendations based on a combination of population data and user-specific data. In some embodiments, meal visualization model 1306 may provide dose recommendations based on user-specific meal data only and may stop using population meal data once sufficient user data is collected.

To rely on user meal data, meal visualization model 1306 may require a minimum number of records of a particular meal. For example, meal visualization model 1306 may require the user has the same meal 3 times, at least 5 times, or at least 7 times. Having additional meal records for each meal helps to improve the accuracy of the dose recommendation due to the larger amount of data, but waiting for additional meals to be consumed may delay providing dose recommendations tailored to the user. In an example, the meal visualization system 1300 may rely solely on population data before the user has eaten the meal, the meal visualization system 1300 may rely on a combination of population data and user-specific meal data when the user has had the meal 1 or 2 times, and once the user has had the meal 3 times, meal visualization model 1306 may rely on the user-specific meal data only in determining the dose recommendation.

Dose recommendation information may be included in output files provided by meal visualization model 1306, or may be stored in a separate database (e.g., additional data sources 1304. Prompt templates provided by prompt generator 1610 (or prompt library 1645, or prompt engine 1651) can be configured with instructions for indicating whether the dose recommendation information should be generated by meal visualization model 1306, or retrieved from a separate database. If to be generated by meal visualization model 1306, then the predefined prompt template may be configured with instructions or algorithms for generating dose recommendations based on the provided meal information and the user's health data.

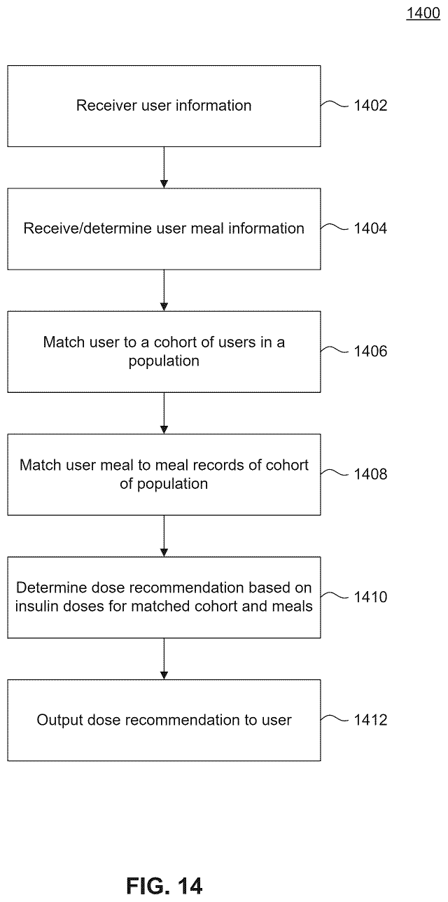

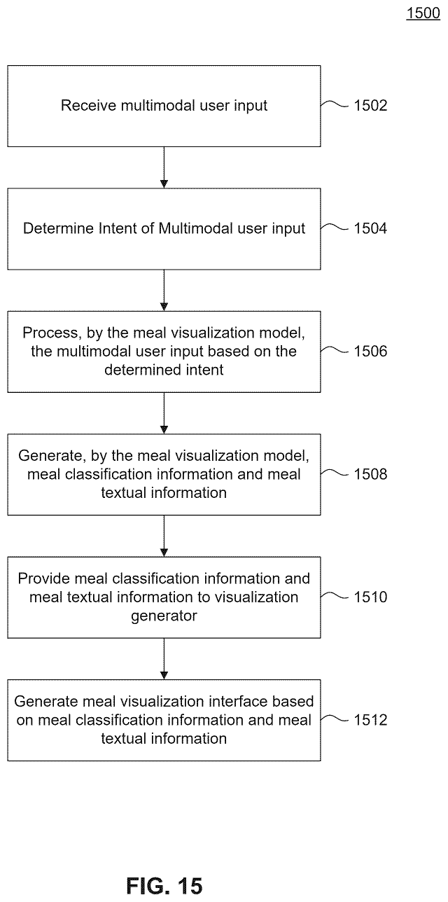

FIG. 14 shows a flowchart of an exemplary method 1400 for providing dose recommendations based on meal data from a population. In some embodiments, method 1400 may be performed upon an initial startup of the meal visualization system 1300 or, in any embodiment when meal visualization model 1306 does not have access to a meal history for the user. As a non-limiting example with regards to FIGS. 1-3, 13, and 16A-16B one or more processes described with respect to FIG. 14 may be performed by a meal visualization model 1306 implemented on one or more devices, including user devices (e.g., data receiving device 120 of FIG. 1, meal visualization device 1310, user device 1302, multi-purpose data receiving device 130, user device 140, and local computer 170), which may include an imaging device or a display device (e.g., meal visualization device 1310 of FIG. 13), and/or remote servers (e.g. remote application server 155, application storefront 160, trusted computer system 180, or other remote servers accessed via cloud 190). In such an embodiment, any of these devices may include suitable components (i.e., a processor) and may execute code in memory to perform certain steps of method 1400 of FIG. 15. While method 1400 of FIG. 14 will be discussed below as being performed by a meal visualization model (e.g., meal visualization model 1306) and/or a meal visualization device (e.g., meal visualization device 1310), other components may store the code and therefore may execute method 1400 by directly executing the code. Accordingly, the following discussion of method 1400 will refer to various components of FIG. 13 as an exemplary non-limiting execution of method 1400. Moreover, it is to be appreciated that not all steps may be needed to perform the disclosure provided herein. Further, some of the steps may be performed simultaneously or in a different order than shown in FIG. 14, as will be understood by a person of ordinary skill in the art.

In 1402, meal visualization model 1306 receives user information (e.g., within a prompt generated from a predefined prompt template), also referred to herein as user characteristics, and in 1404, meal visualization model 1306 receives user meal information (e.g., within a prompt generated from a predefined prompt template). In some embodiments, user information comprises any information accessible from additional data sources 1304 and can include user insulin on board, dosing history (e.g., time and amount of previous doses, current glucose level, historical glucose levels, rate of change). User meal information may refer to historical meal information associated with the user and in some embodiments, when the meal visualization system 1300 is initialized, user meal information may not include sufficient (or any) meal information for meal visualization model 1306 to generate dose recommendations. In some embodiments, step 1404 may include determining user meal information, such as via any received multimodal inputs. Meal records for a population of users may be stored in a database, wherein the meal records include meal information, glucose data associated with the meal information, and insulin data associated with the meal information.

In some embodiments, 1404 may include a step for meal visualization model 1306 to determine whether there is sufficient meal information in user meal information to generate a dose recommendation for the user. For example, instructions within a prompt provided by a prompt generator (e.g. prompt generator 1610, prompt library 1645, or prompt engine 1651) may include thresholds and/or conditions to be satisfied in order for meal visualization model 1306 to proceed with the dose recommendation. In some embodiments, the prompt may include instructions for meal visualization model 1306 to retrieve threshold information from an external source for determining whether there is sufficient meal information. If there is insufficient information, instructions in the prompt may cause meal visualization model 1306 to next determine whether to retrieve cohort information for use as inputs for generating the dose recommendation for the user.

In 1406, meal visualization model 1306 is configured to match the user with a cohort of users of the population by a machine learning model based on the user information. For example, meal visualization model 1306 may perform the matching based on one or more characteristics of the user including but not limited to user age, user medical condition, user medical history, user location, and user preference.

In 1408, meal visualization model 1306 matches the user meal information to meal records of the cohort of users of the population. Meal visualization model 1306 may utilize the user meal information to further refine the matching process. For example, after identifying a cohort of users, meal visualization model 1306 may then utilize user meal information identify a subset of the cohort based on matching or similar meal information.

In 1410, meal visualization model 1306 may determine insulin dose recommendations based at least in part on the insulin data associated with the meal records of the cohort of users of the population that match the user meal information 1410.

In 1412, meal visualization model 1306 may output the insulin dose recommendation is output to a user device (e.g., data receiving device 120) associated with the user. As noted above, the user device can be or include an insulin pump, insulin injection pen, or a smart insulin pen cap.

FIG. 15 is a flowchart depicting method 1500 comprising steps for performing meal visualization analysis based on multimodal inputs according to an embodiment. As a non-limiting example with regards to FIGS. 1-3, 13, and 16A-16B one or more processes described with respect to FIG. 15 may be performed by a meal visualization model implemented on one or more devices, including user devices (e.g., data receiving device 120 of FIG. 1A, meal visualization device 1310, user device 1302, multi-purpose data receiving device 130, user device 140, and local computer 170), which may include an imaging device or a display device (e.g., meal visualization device 1310 of FIG. 13), and/or remote servers (e.g. remote application server 155, application storefront 160, trusted computer system 180, or other remote servers accessed via cloud 190). In such an embodiment, any of these devices may include suitable components (i.e., a processor) and may execute code in memory to perform certain steps of method 1500 of FIG. 15. While method 1500 of FIG. 15 will be discussed below as being performed by a meal visualization model (e.g., meal visualization model 1306) and/or a meal visualization device (e.g., meal visualization device 1310), other components (i.e., memories) may store at least a portion of the code and therefore may execute method 1500 by directly executing the portion of the code. Accordingly, the following discussion of method 1500 will refer to various components of FIG. 13 as an exemplary non-limiting execution of method 1500. Moreover, it is to be appreciated that not all steps may be needed to perform the disclosure provided herein. In particular, steps relating to determining the intent of the user are generally considered optional as the intent may already be known based on, for example, how the user interacted with a particular graphical user interface. For instance, selecting icon 8020 in FIG. 8B and inputting image data according to FIG. 8A, the intent of inputting meal information is readily apparent. Further, some of the steps may be performed simultaneously or in a different order than shown in FIG. 15, as will be understood by a person of ordinary skill in the art.

In 1502, meal visualization device provides multimodal user input to a meal visualization model, which in some embodiments, may include receiving image data and other user input (referred to elsewhere herein as additional user input). In some embodiments, the multimodal user input is configured as part of a predefined prompt template that includes placeholders into which different types of user input are to be inserted. The multimodal user input may be provided via components of a meal visualization device 1312, such as an imaging device (i.e., a camera) and touch-based user interface (i.e., a touch screen). As noted above, multimodal user input may comprise any combination of a textual data, such as a user query, image data, audio data, and visual data. Further details on user input are discussed with respect to FIG. 13, especially with respect to user multimodal input 1302A and user query 1302B. Examples of an imaging device include a camera of the meal visualization device.

The multimodal user input, including any textual data, image data, audio data, and video data, may be provided as inputs to meal visualization model 1306. An example of user input may include a user query, for example user query 1302B of FIG. 13, that includes a request for information associated with the data provided in the multimodal user input. A further example of a user input is a prompt. Many other examples are discussed with respect to FIG. 13, as well as FIGS. 8A-12B.

In 1504, meal visualization model 1306 receives the user input as part of a prompt and, in some embodiments, determines the intent of the multimodal user input. In some embodiments, determining the intent of the multimodal user input comprises detecting additional user input, such as a user query (e.g. user query 1302B of FIG. 13, also referred to herein as query data), within the multimodal user input and determining the intent of the user query in relation to any other data provided with the multimodal user input. For example, multimodal user input may include image data such as a picture of a meal and additional user input, such as a query regarding the picture. In some embodiments, the prompt may include the intent. In other embodiments, the intent may be readily apparent from the context in which the prompt is being made. In other words, in some embodiments, the intent may be predetermined. Here “intent” is a reference to the information the meal visualization model 1306 should output, usually related to one or more meal components and/or glycemic impact of the one or more meal components. For example, intent may be to provide visualization generator 1312 with the necessary information to provide any of the graphical user interfaces of FIGS. 8A-12B. In some embodiments, intent dictates the structured output file that is output by meal visualization model 1306.

In 1506, meal visualization model 1306 processes the multimodal user input based, in some embodiments, on the determined intent. Processing the multimodal user input can include determining whether additional inputs (in addition to the data provided by the multimodal user input) are needed for generating a response to the determined intent of the user query. For example, meal visualization model 1306 may determine that additional user data, such as analyte data, glucose levels, medical history, or meal history, is needed as inputs to generate a response that is tailored to the user query. Other additional user data and additional data that may be used by meal visualization model 1306 are discussed with respect to FIG. 13. Meal visualization model 1306 may be configured to access or otherwise retrieve the determined additional inputs to provide with the multimodal user input. Alternatively, determining whether additional inputs are needed may be performed by the prompt generator (e.g. prompt generator 1610, prompt library 1645, or prompt engine 1651). Alternatively, additional inputs may be received by the meal visualization device 1306 from a user interface and provided as part of the prompt. Various sources for the additional user data and additional data are discussed with respect to FIG. 13 (for example, additional data sources 1304). In embodiments where the intent is predetermined, step 1506 may involve receiving the additional user data, and optionally additional data, i.e. without the further steps of having to determine which data are needed for generating a response. In other words, the additional user data and/or additional data may be predetermined, for instance the additional user data and/or additional data may be defined in the prompt.

In 1508, meal visualization model 1306 generates a response to the multimodal user input. In some embodiments, the response includes meal classification information and meal textual information. The meal classification information may include identifiers associated with meal objects detected within the multimodal user input (i.e., identified meal components). For example, meal visualization model may be configured to perform image recognition of image (or video) data, audio recognition of audio (or video data), and word recognition of text data, and associate meal component identifiers within each detected meal object within the multimodal user input. Examples of meal component identifiers include names of identified meal objects, such as grilled chicken, mashed potatoes, or white rice. Meal textual information includes automatically generated text related to the detected objects within the multimodal user input. In some embodiments, the meal textual information is generated based on the meal component identifiers. In some embodiments, the meal visualization model may also output glycemic impact predictions (i.e., glycemic impact values or scores), as further discussed elsewhere herein. Other outputs provided by meal visualization model 1306 are shown and discussed with respect to FIGS. 8A-12B.

In some embodiments, visualization parameters may be assigned to each meal component identifier. The visualization parameters may be based on a prompt or may be included in a look-up table for each meal component identified. For example, meal component identifiers can be associated with a different glucose impact values and the meal classification information further includes color-coded tags that are associated with each of the different glucose impact values (e.g. a red or orange tag can be associated with a high glucose impact value, a yellow tag can be associated with a moderate glucose impact value, and a green tag can be associated with a low glucose impact value). The meal visualization parameters may be utilized by a meal visualization interface (i.e., a graphical user interface for display on meal visualization device 1310), in particular to generate appropriate graphical user interfaces based on the meal classification information and the meal textual information provided by meal visualization model 1306. The meal visualization interface may be generated and displayed within a medical application executing on the meal visualization device 1310. The medical application may be one of: a monitoring application of a continuous glucose monitor (CGM) (or any other analyte), a standalone application, a standalone application which is not a monitoring application of a continuous glucose monitor (CGM) (or any other analyte), and an insulin delivery application.

In some embodiments, a user interface (i.e., a graphical user interface) may include various interface components that are configured to display graphical elements provided by meal visualization model. Example of graphical elements include text boxes that are generated to display information associated with the meal classification information and the meal textual information. For example, graphical elements can be displayed based on their respective color-coded tags. Color-coded tags may be implemented as any visual indicator, which can include icons, images, or components of the interface components. Other interface components and suitable graphical elements are displayed in FIGS. 8A-12B. In general, graphical elements may include interactive elements (e.g. buttons, checkboxes, radio buttons, switches/toggles, slides, dropdowns/select menus, text fields, and the like), container/structure elements (e.g. cards, tabs, sidebars, dialogs, popups, tooltips, lists, tables, galleries), navigation elements (e.g. navigation bars, step indicators, and the like), informational elements, (e.g. icons, images, avatars/profile pictures, labels/captions, badges, process indicators, status indicators, alerts, banners, and the like), and more

In 1510, meal visualization model provides the generated outputs to a visualization generator 1312, which is configured to display the meal classification information and the meal textual information, based on any meal visualization parameters.

In 1512, meal visualization generator 1312 generates the meal visualization interface based on the meal classification information and the meal textual information. The meal visualization interface may include various interface components that are configured to display graphical elements associated with the meal classification information and meal textual information, provided the meal visualization model 1306. For example, the outputs provided by meal visualization model 1306 may include visualization parameters that cause the meal visualization interface to display the meal component identifiers and meal textual information within the meal visualization interface. Any of the components shown in the interfaces of FIG. 8A to FIG. 12B, as well as those listed above, may be displayed as a result.

Meal visualization interface, i.e., the graphical user interface provided by visualization generator 1312, may include additional interface components (i.e., visual interface components) for accessing and displaying other information provided by meal visualization model. For example, the meal visualization system may receive information from another system, such as a continuous analyte monitor (i.e., analyte sensor 104 or sensor control device 102) or an EHR database (e.g. provided by a remote server, as discussed elsewhere herein). The meal visualization model may be configured to receive analyte data from the continuous analyte monitor or other system and, in some embodiments, an additional real-time query as inputs and generate an analysis based on the provided inputs. The analysis may be configured with visualization parameters that cause the meal visualization interface to display the analysis in an interface component.

FIG. 16A is a block diagram of system 1600A including meal visualization model 1306, prompt generator 1610, and visualization generator 1312. Prompt generator 1610 is configured to generate a prompt based on a predefined prompt template that can be stored in prompt generator 1610. Prompt generator 1610 is configured to receive any combination of inputs, including information provided by visualization generator 1312 such as media data 1602, text input 1604, and queries/chat data 1606 as well as user data provided from additional data sources 1304. Other possible inputs are discussed with respect to FIGS. 8A-13. Because the output of meal visualization model 1306 is utilized by visualization generator 1312 to generate meal visualizations, the input to meal visualization model 1306 is configured to provide predictable and consistent outputs that can be processed by visualization generator 1312 for generating interfaces for display. In some embodiments prompt generator 1610 may be implemented on the same device as meal visualization model 1306 or on a separate device. In some embodiments, prompt generator 1610 may be implemented on a user device (e.g. meal visualization device 1310, user device 1302, data receiving device 120, multi-purpose data receiving device 130, user device 140, or local computer 170) or on a remote device such as a remote server (e.g. mote application server 155, application storefront 160, trusted computer system 180, other remote servers accessed via cloud 190, or other backend system).

That is, it is known that machine learning models, such as meal visualization model 1306, may generate different outputs even when given the same input prompt due to the randomized and sampling techniques that such models use for generating its output. This is especially the case for pre-existing/pre-trained machine learning models such as large language models. This approach typically allows for a natural variation in responses, which helps make the outputs seem more human-like and less repetitive. However, this randomness or variation in outputs is not needed in scenarios where the output of the model is to be processed by another system (e.g., as is the case for meal visualization model 1306 and visualization generator 1312) and consistent output is desired.

The output of the meal visualization model 1306 is identified meal components 1622 and glycemic impact prediction 1624. Prompt generator 1610 can utilize a predefined prompt template or generate a prompt template based on one or more output rules for ensuring that the output of the model is consistent. An example output rule include setting a temperature parameter of the model to below a predefined threshold. The temperature parameter controls the level of randomness when meal visualization model 1306 selects a next word or part of output when generating output electing a next word or part of the output. A temperature rule for prompt generator 1610 can set a predefined threshold for the temperature level (e.g., close to or equal to 0) to ensure that higher probability words are consistently selected which increases consistency of the output. Other rules include a structured output rule, an explicit output rule, and a few-shot prompt rule. A structured output rule for prompt generator 1610 can define a specific output format (e.g., JSON, XML), which can include specific keys and data types to be provided within the output format. This is particularly useful for the visualization generator 1312. An explicit output rule can define the specific output to be included in the output, such as the structure of the output (e.g., tabular data, numbered output, list data). A few-shot prompt rule can define an example output within the prompt to illustrate the desired format and structure of the output. In any event, the one or more output rules should include at least a rule for outputting the identified meal components 1622 and a rule for outputting the glycemic impact prediction 1624.

In some aspects, prompt generator 1610 may store one or more predefined prompt templates generated based on one or more of the output rules. For example, predefined prompt templates may be structured with a low temperature, define a specific output format (e.g., JSON, XML), provide explicit instructions on the output content and structure (e.g., specific number of items to be included in the output), and/or a few-shot example of the output that is desired to be output by meal visualization model 1306.

Prompt templates can be configured to include placeholders for inserting certain types of content. Prompt generator 1610 is configured to receive input data, such as media data 1602, text input 1604, queries/chat data 1606, and user data from additional data sources 1304. Other placeholders may be used for other possible inputs, for example any of those discussed with respect to FIGS. 8A-13. Prompt generator 1610 can then determine how to insert the received input data into appropriate placeholder locations within the predefined prompt template.

Examples of input that can be inserted into the predefined prompt templates include media data 1602, text input 1604, and queries/chat data 1606 as well as user data provided from additional data sources 1304. Media data 1602 is an embodiment of user multimodal input 1302A, provided by user device 1302. Text input 1604 and queries/chat data 1606 are embodiments of user query 1302B, provided by user device 1302. However, it should be appreciated that other data may be inserted, including the image data, additional user input and additional input discussed with respect to FIG. 13. Examples of media data 1602 include multimedia data provided by user device 1302 (e.g., as user multimodal input 1302A). Multimedia data includes images of meals or menus, video of meals or menus, and audio data describing a meal including its meal components or describing menu items on a menu. As shown in FIG. 16A, media data 1602 can be provided via an interface provided by visualization generator 1312. Examples of text input 1604 include text-based data provided by user device 1302 (e.g., user query 1302B). Text-based data includes descriptions of meals and descriptions of menus. Examples of queries/chat data 1606 can include text provided in an unstructured, conversational format including queries regarding meals, questions regarding meals and glycemic impact, and other user-related impacts, to name a few examples. Other examples are discussed throughout the specification, particularly with respect to FIGS. 8A-12B.

User data provided from additional data sources 1304 includes user data 1304A, continuous analyte data 1304B, additional multimodal meal data 1304C, and backend system data 1304D. This data is discussed above with respect to FIG. 13. Prompt generator 1610 can be configured to determine whether information from additional data sources 1304 needs to be inserted into the prompt. This determination may be based on inputs provided by any one of media data 1602, text input 1604, queries/chat data 1606. For example, queries/chat data 1606 may include a query that requests the predicted glycemic impact score for a user based on a meal depicted within image data. Prompt generator 1610 can be configured to determine that user data, such as the continuous glucose data 1304B associated with the user, is needed as part of the prompt in order to generate a responsive output. However, in other embodiments, the prompt generator 1610 automatically retrieves data from the additional data sources 1304.

Prompt generator 1610 is configured to receive any combination of these inputs, process the received inputs, and generate a prompt for meal visualization model 1306 based on the received inputs. In some aspects, prompt generator 1610 can identify appropriate placeholders within a predefined prompt template, and insert the received inputs into the identified placeholders. Data type may be used to identify a particular placeholder. As an example, one placeholder may identify image data-types and another placeholder may identify video data types. Accordingly, prompt generator 1610 will determine whether received input includes image data and video data, and if so, insert the image input into the image data-type placeholder and the video input into the video data-type placeholder. Different predefined prompt templates may be used based on intent, as discussed with respect to FIG. 14. Prompt generator 1610 provides the generated prompt to meal visualization model 1306.

In some embodiments, as previously mentioned, meal visualization model 1306 may be a large language model. In some instances, the large language model may be a customized multimodal large language trained with multiple sub-models to perform different functions of processing different types of input and generating output for use by visualization generator 1312 (as discussed above). In some embodiments, meal visualization model 1306 may be implemented as a pre-trained/pre-existing multimodal large language models (LLM), such as ChatGPT-4, Claude, Gemini, or Llama. Other versions of ChatGPT, such as ChatGPT-5 and variations of ChatGPT-4 (e.g. 4 omni and others) may also be used.

When the meal visualization model 1306 is implemented as a pre-trained/pre-existing multimodal large language model 1306, such pre-trained/pre-existing large language models may only handle multimodal input with the use of extensions or plug-in components, and thus the meal visualization model 1306 may be configured with one or more extensions or plug-in components, as shown in FIG. 16A, for performing different functions for processing the received prompt template. Alternatively, when not using a pre-trained/pre-existing meal visualization model, the components shown in FIG. 16A form the sub-models for the meal visualization model 1306.

Media recognition component 1612 is configured with capabilities for performing image recognition, audio recognition, and optical character recognition (OCR). Meal visualization model 1306 can route detected media input to media recognition component 1612 for appropriate processing based on the media type (i.e., image, video, or audio). Media recognition component 1612 can utilize image recognition for analyzing and detecting objects in images or video. Media recognition component 1612 can identify objects such as different meal component or meal types within an image. Media recognition component 1612 can utilize OCR for interpreting text within images or videos, such as images or videos of menus.

Natural language component 1614 is configured with capabilities for processing and generating human language provided in textual format. Natural language component 1614 can process text inputs in prompts, determine intent within the text input, and generate corresponding text in response to the determined intent and any other inputs provided in the prompt and in accordance with any output rules included within the prompt. An example utilization of natural language component 1614 includes text describing a meal and/or meal components, a question involving potential impact of a meal or a user-specific question, such as glucose values associated with the user.

Media output component 1616 is configured with capabilities for generating media such as images or videos. A pre-existing implementation of media output component 1616 is DALL-E but is not limited to this implementation. Media output component 1616 can implement an image generation tool that uses text descriptions (e.g., provided via a prompt from prompt generator 1610) to create new images as determined based on the prompt and any associated output rules.

Knowledge retrieval component 1618 is configured to access external curated knowledge bases and search engines if additional information is needed for more specific or up-to-date information that may not be part of the pre-trained model. Prompts provided by prompt generator 1610 may be configured to trigger functions of knowledge retrieval component 1618 to access additional information. As one example, a prompt that includes an image could include language within the prompt to request meal visualization model 1306 to retrieve similar images from other knowledge bases (e.g., a recipe database, a nutrition database, a food database). Meal visualization model 1306 can then detect meal components within the image input based on the identification of the similar images and the meal components that correspond to those retrieved images.

File interpretation component 1620 is configured to perform document Analysis such as by processing various file types such as PDFs, CSVs, a document, that may be included in the prompt. File interpretation component 1620 can extract and interpret data from uploaded documents, including performing analysis or summarizing content.

Meal visualization model 1306 can be configured to collect outputs from any triggered component and generating the output based on the received outputs and output rules associated with the provided prompt. As discussed above, the prompt templates are configured to cause meal visualization model 1306 to provide consistent and structured outputs that can be processed by visualization generator 1312. This structured output includes identified meal components 1622 and a glycemic impact prediction 1624. In some embodiments, the structured output may include a JSON or XML file, and can be structured specifically as defined by the prompt.

Identified meal components 1622, which may form part of the meal classification information discussed with respect to FIG. 15, can include any meal components detected by one or more components of meal visualization model 1306 and related metadata associated with each detected component. The meal component metadata may be provided as defined by output rules in the prompt template or may be obtained via a look-up table based on the meal component identifier (e.g. the meal component name). The metadata associated with each meal component may specify any one of the format, layout, position, visual component, that is associated with presenting the meal component by visualization generator 1312. As one non-limiting example, identified meal components 1622 may indicate breaded tilapia, pasta, marinara sauce, and vegetables as respective meal components. Metadata for the breaded tilapia can indicate the visual component that is associated with this meal component (e.g., meal component identifiers 9002A), the color of the component (e.g., red, green, yellow), and the position of the component within the visualization. Metadata may also be provided for the pasta, marinara sauce, and vegetables with corresponding details associated with the visual component, color, and position.

In some aspects, metadata associated with identified meal components 1622 is determined based on user data provided by additional data sources 1304 as part of the prompt. In other words, the metadata associated with identified meal components 1622 may be dynamic for the user. This dynamically determined metadata may be used to customize the user interface for the user, including based on analyte data. For example, the color of the component may indicate the level of glycemic impact on the user, which is determined by meal visualization model 1306 based on both the meal image of the breaded tilapia but also glucose data associated with the user and that can be provided by additional data sources 1304, such as a continuous analyte monitor (e.g., a glucose sensor such as analyte sensor 104 or sensor control device 102).

Glycemic impact prediction 1624 can include a predicted glycemic impact (e.g., as represented by a glycemic impact score, also referred to herein as a glucose impact value and similar) of the identified meal components 1622 on a glucose level of the user. In some embodiments, the glycemic impact prediction 1624 may be based on current glucose levels or any other user medical information. A suitable algorithm may be used to determine the glycemic impact prediction, as discussed elsewhere herein. Prompt generator 1610 may be configured to determine when to include current glucose levels or any other user medical information in placeholders of the prompt template. Glycemic impact prediction 1624 may also include metadata for determining the visualization (i.e., interface component) of the predicted glycemic impact by visualization generator 1312. The glycemic impact metadata may be provided as defined by output rules in the prompt template or may be obtained via a look-up table based on the glycemic impact prediction. Examples of the metadata for glycemic impact prediction 1624 can include any one of the format, layout, position, visual component, that is associated with presenting the glycemic impact prediction 1624. Examples of visual interface component can indicate the visualization type or widget (e.g., toolbar, graph, bar chart) in which glycemic impact prediction 1624 is presented by visualization generator 1312. Examples of format include the colors (e.g., visualization, line, graph), lines and line properties (e.g., line type, line width, line color).

The identified meal components 1622 and/or glycemic impact prediction 1624 may form part of the meal textual information discussed at FIG. 15. This is because the identified meal components 1622 and glycemic impact prediction 1624 can be used to determine a structured output file. The structured output file is a type of text file, i.e. JSON. As mentioned, metadata may be included as a structured output file based on the prompting provided to meal visualization model 1306. In other words, the prompt template is designed so that the metadata generated by meal visualization model 1306 results in generating the desired visualizations of the inputs by visualization generator 1312. Visualization generator 1312 can receive the structured output from meal visualization model 1306 and generates visualizations (e.g., graphical user interface 9000C, graphical user interface 1000A, graphical user interface 1100A) based on the identified meal components 1622 and a glycemic impact prediction 1624, and associated metadata.

FIG. 16B is a block diagram of system 1600B depicting an additional embodiment for implementing meal visualization model 1306. In some embodiments, system 1600B can be implemented in addition to system 1600A. In some embodiments, system 1600B represents an alternative implementation of system 1600A for providing model predictions using meal visualization model 1306. System 1600B includes an application 1630 in communication with a backend system that can include meal visualization model 1306 and validation engine 1650. Application 1630 is a software application implemented on a user device, such as meal visualization device 1310, user device 1302, data receiving device 120, multi-purpose data receiving device 130, user device 140, or local computer 170. Application 1630 may be downloaded from application storefront 160. Remote servers such as remote application server 155, application storefront 160, trusted computer system 180, or other remote servers accessed via cloud 190, are examples of backend systems. As discussed with respect to FIG. 13, meal visualization model 1306 may be implemented at least partly on a remote server.

In embodiments, application 1630 is configured to receive (or otherwise generate) and transmit multimodal input 1632 (the line labeled “Meal” in FIG. 16B) and to receive and display meal visualizations 1634 (the line labeled “Result” in FIG. 16B). The multimodal input 1632 is received at the meal visualization model 1306 and transmits meal visualizations. Multimodal input 1632 may include image data and additional user input, and in some embodiments additional input (as discussed with respect to FIG. 13 and FIG. 15). Meal visualizations 1634 may include any data for generating the meal visualization interface, i.e. a graphical user interface (as also discussed with respect to FIG. 13 and FIG. 15).

In embodiments, application 1630 can be configured to communicate directly with additional data sources 1304 such as glucose monitoring devices (e.g., analyte sensor 104, sensor control device 102) to receive glucose information. Glucose monitoring devices can be restricted to communicate only with application 1630.

In embodiments, application 1630 can be configured to communicate with a dedicated glucose monitoring application configured to communicate with glucose monitoring devices (e.g., analyte sensor 104, sensor control device 102) to receive glucose information. In such embodiments, application 1630 is configured with security protocols for communicating the dedicated glucose monitoring application to protect the privacy of the user data provided by glucose monitoring devices. Application 1630 may be implemented with a handshake procedure for communicating with the dedicated glucose monitoring application. Other examples include establishing a secure memory location on the user device on which application 1630 and the dedicated glucose monitoring application are installed and can be used for securely sharing data between the applications. In some embodiments, application 1630 may implement a request-response procedure for communicating with the dedicated glucose monitoring application, which may perform authentication of the request from application 1630.

In embodiments, meal visualization model 1306 can be implemented with prediction guardrails component 1641 and a prediction framework 1642. Prediction guardrails component 1641 is configured to receive multimodal input 1632 from application 1630 and prediction framework 1642 is configured to generate meal visualizations 1634 for display by application 1630.

Prediction guardrails component 1641 is configured to perform safety checks on multimodal inputs, such as multimodal input 1632, from application 1630. Prediction guardrails component 1641 is configured to sanitize inputs prior to provision to an AI model (i.e., meal prediction engine 1643 of meal visualization model 1306). Examples of sanitizing inputs include performing object recognition on multimodal inputs to detect and block requests that include restricted content (i.e., faces, personal text data), illegal or improper/inappropriate content for models, and ensuring that the multimodal inputs conform to cybersecurity or network security rules and protocols.

Prediction guardrails component 1641 is communicatively coupled with application 1630 which provides the multimodal inputs, and with meal prediction engine 1643 configured to perform processing on the sanitized inputs. In embodiments, prediction guardrails component 1641 is configured with specific regulatory and operational safety standards and further configured to enforce input compliance with the standards.

In embodiments, prediction guardrails component 1641 is configured to evaluate multimodal inputs against one or more content validation protocols, which may include rule-based pattern matching, format verification, content classification, or other heuristic or algorithmic techniques. Based on such evaluation, prediction guardrails component 1641 can filter, redact, or otherwise modify multimodal input to remove restricted or otherwise non-conforming elements prior to passing the sanitized input to the AI model (i.e., meal prediction engine 1643 of meal visualization model 1306). In embodiments, prediction guardrails component 1641 may be configured with image recognition functions in order to perform detect any restricted content such as faces, illicit or illegal content, and any content unrelated to meal visualizations that would include objects unrelated to meals (e.g., pictures of buildings, pets, or landscapes).

Prediction framework 1642 is configured with meal prediction engine 1643 (i.e., an AI model), error check engine 1644, prompt library 1645, and monitoring engine 1646. In embodiments, prediction framework 1642 receives sanitized multimodal input from prediction guardrails component 1641 and generates corresponding visualization components for display by application 1630.

Meal prediction engine 1643 is communicatively coupled to prediction guardrails component 1641 and configured to process the multimodal input received from prediction guardrails component 1641. Meal prediction engine 1643 is configured to generate output based on the multimodal input. Meal prediction engine 1643 is configured to detect meal objects within the multimodal input, which can include image recognition techniques for images and video and audio recognition techniques for audio input. In some embodiments, the output includes the detected meal objects organized into meal classification information and meal textual information, as discussed further with respect to FIG. 15. In embodiments, meal prediction engine 1643 is configured to provide output by associating the detected meal objects, including meal classification information with visual components such as visual meal identifiers. Meal prediction engine 1643 may be implemented using pre-existing/pre-trained machine learning model, such as a large language model, particularly a multimodal large language model. Examples of suitable existing models include ChatGPT. Any of the other pre-existing models discussed herein may be used. Alternative, a model containing various sub-models corresponding to the components shown in meal visualization model 1306 of FIG. 16A may be used.

Output from meal prediction engine 1643 represents a prediction of the meal objects and/or the meal within the multimodal input (i.e., akin to the identified meal components 1622 of FIG. 16A) as well as a predicted glucose impact value associated with each meal object and/or meal (i.e., akin to the glycemic impact prediction 1624 of FIG. 16A). In embodiments, meal prediction engine 1643 is configured with a glucose impact algorithm for generating the glucose impact value for the meal based on historic glucose values for a predefined period after the meal (e.g., 1 to 4 hours after the meal), calculating a peak glucose value for the historic glucose values for the predefined period after the meal, performing a calculation for adjusting delta to peak value, and then binning the adjusted delta to peak value to generate the predicted glucose impact value of the meal.

In some embodiments, meal prediction engine 1643 is configured to collect historic glucose values for a predetermined time period after a meal. Example predetermined time periods include 1 hour, 2 hours, and 3 hours. The length of the predetermined time period can be set to a default value for all users and then personalized for each user as more user information is provided to the glucose impact algorithm. Personalizing the predetermined time period can result in collecting glucose values for varying time periods to provide more accurate predictions for each user. In some embodiments, a historical analyte data range is defined as a post-meal period. The post-meal period can range from the time of the initial pre-meal analyte level value (e.g., fifteen minutes prior to or after a meal entry, thirty minutes prior to or after a meal entry, or one hour prior to or after a meal entry) to a time of the peak analyte value following the meal (e.g., within two-hours after the meal entry, within three-hours after the meal entry, within four-hours after the meal entry). In some embodiments, the end of the post-meal period may be one of the two following events which occurs first: (1) a first analyte (e.g., glucose) reading within three hours from the initial analyte level value, or (2) a last analyte (e.g., glucose) reading prior to a new meal entry being inputted.

Meal prediction engine 1643 can determine an analyte level excursion by, for example, by subtracting the initial analyte level value from a peak analyte level value for a post-meal time period. Meal prediction engine 1643 can determine the meal impact value of a particular meal based on the analyte level variance. In some embodiments, the meal impact value can be generated for particular meal after the end of the post-meal period. In some embodiments, the meal impact value for each of one or more meal events is calculated based on a glucose response based on data indicative of a glucose level associated with each meal event. In some embodiments, a meal impact value is not generated for the meal if certain thresholds conditions are met. A first threshold condition, for example, can be less than a minimum number of analyte readings (e.g., eight glucose readings) within the post-meal period and/or no peak analyte value was detected before a predetermined time window (e.g., a three-hour time window) elapsed from the time of the meal entry. Further, in some embodiments, a second threshold condition can be when the post-meal period is less than a predetermined time period (e.g., two hours). In some embodiments, if a new meal entry is inputted within a predetermined time period following a previously inputted meal entry (e.g., within three hours following an existing logged meal entry), then a peak analyte value cannot be detected, and the meal impact value cannot be generated.

According to some embodiments, assigning or calculating the meal impact score can further take into account certain physiological conditions present in the user before the meal is consumed. More specifically, certain individuals with diabetes can have a high initial analyte level (e.g., glucose level) value prior to consuming a meal. For example, some individuals with Type 2 diabetes, who still have the ability to manufacture insulin to metabolize glucose, may frequently exhibit a high pre-meal glucose level. Endogenous insulin present in those individuals prior to consuming a meal can thus impact the response to the meal and may attenuate the analyte level excursion value (also referred to as “PeakDelta”). In embodiments, the peak delta can be defined as the glucose level difference between a pre-meal glucose level and the peak glucose level in a post-meal time period, based on glucose readings such as from a continuous glucose sensor, within a predetermined duration (e.g., 15 minutes, one hour, ninety minutes, two hours, three hours, etc.) or for a predetermined range (e.g., up to 15 minutes, one to three hours, up to four hours, etc.) after meal entry.

Meal prediction engine 1643 is further configured to calculate the peak glucose value for the predetermined time period based on the collected historical glucose values. In embodiments, calculating the adjusted delta to peak value from the peak glucose value comprises measuring how much glucose levels rise from a reference level (such as baseline or pre-event level) to a peak amount. After calculating the peak amount, normalizing or adjusting that measure based on a contextual factor (i.e., factor personalized to the user) such as individual baseline or timing. The baseline or pre-event level can be the pre-meal glucose value (e.g., the glucose value prior to the predetermined period). Other factors that can be included in the calculation include an elapsed time to peak, individual insulin sensitivity, or total carbohydrate load of the meal. In some embodiments, the actual glucose impact value for a meal can replace the predicted glucose impact value as depicted within a visual component (e.g., glucose impact component 8110A, glucose impact component 8110B), i.e., after the meal has been consumed.

In some embodiments, meal prediction engine 1643 can determine a meal-time dose (e.g., a dose amount to be titrated) based on a predicted carbohydrate load of the meal. After detecting meal components of a meal, meal prediction engine 1643 can be trained to further calculate a predicted carbohydrate load of the detected meal components and detected portion size of each meal component. In some embodiments, meal prediction engine 1643 may calculate the predicted carbohydrate load based on a request to provide a meal-time dose. For example, meal prediction engine 1643 may map detected meal components to a component library that maps meal components to carbohydrate amounts, based on their portion size.

In embodiments, to account for a higher initial analyte level for certain users, an adjustment can be applied to the PeakDelta value. For example, the PeakDelta value can be adjusted according to the following example equation:

$$PeakDeltaAdj = PeakDelta + f ( {G}_{premeal} )$$

The PeakDelta value can represent the analyte level excursion value, or the difference between the peak analyte level value following a meal (e.g., peak glucose level) and the initial analyte level prior to the meal (e.g., pre-meal glucose level). In some embodiments, Gₚᵣₑₘₑₐₗ is the glucose level at the timestamp prior to the meal tag timestamp. In other embodiments, Gₚᵣₑₘₑₐₗ is the minimum glucose level prior to the meal tag timestamp within a predetermined duration from the meal entry (e.g., 15 minutes, one hour, ninety minutes, two hours, three hours, etc.) or for a predetermined range (e.g., up to 15 minutes, one to three hours, up to four hours, etc.). In some examples, this the pre-meal glucose level is the lowest glucose level within the predetermined duration/range.

In addition, according to some embodiments, f(Gₚᵣₑₘₑₐₗ) can be represented by the following linear function:

$$F ( {G}_{premeal} ) = a * {G}_{premeal} + b$$

Variables, a and b, can be constants determined based on simulations of a population of virtual Type 2 diabetic patients that consumed a variety of meals including varying carbohydrate amounts, fat content, and protein content. In other embodiments, variables, a and b, can be constants determined from in vivo data. Those of skill in the art will appreciate that other methods for determining variables, a and b, using one or more of simulated data, in vivo data, population data, or other test data can be utilized. The first variable, a, can be multiplied with Gₚᵣₑₘₑₐₗ, while the second variable, b, can be added to the product of Gₚᵣₑₘₑₐₗ and a. In some embodiments, for example, a=0.4 and b=−50 mg/dL.

According to some embodiments, if data is available for multiple instances of the same meal (or same type of meal) eaten at different times/days, a weighted average of the variables, a and b, can utilized in calculating the adjusted analyte level excursion value, PeakDeltaAdj. In particular, the Gₚᵣₑₘₑₐₗ and peak glucose values can be associated for multiple instances of the same meal. Additionally, in some embodiments, the values for multiple instances of the same meal can be further grouped by meal period (e.g., breakfast, lunch, or dinner). For each meal with more than one instance, variables, a and b, can be estimated as aₑₛₜ and bₑₛₜ. For meals with associated aₑₛₜ and bₑₛₜ values, a weighted average calculation for each parameter can be performed, wherein the weighting is more for meals with more instances. Subsequently, the PeakDeltaAdj value can be determined based on the latest weighted average parameters for a particular user.

In embodiments, a logistic function can be utilize to minimize the influence of Gₚᵣₑₘₑₐₗ when PeakDelta is low. This can serve to reduce improper scoring for small meals with minimal analyte level excursion values. One example of a logistic function is shown by the following equations:

$$k = \frac{1}{1 + {e}^{x * ( PeakDelta - y )}}$$

$$PeakDeltaAdj = k * ( a * {G}_{premeal} + PeakDelta \text{+/-} b ) + ( 1 - k ) * PeakDelta$$

In the above equations, a, b, x and y are constants. Variables a and b are discussed above. Variables x and y represent constants that can be adjusted for the particular logistic function in calculating the adjusted peak delta value. Example ranges for variable x include=1.000<x<−0.100. Example ranges for variable y include 1.0<y<2.0 or 1.1<y<1.9.

In embodiments, binning the adjusted delta to peak comprises binning aggregating or grouping values into discrete time intervals, or “bins.” Meal prediction engine 1643 is configured to receive a continuous stream of adjusted delta to peak values and via binning, configured to convert the continuous stream of values into a smaller set of intervals, with each bin containing a single aggregated adjusted delta to peak value, such as a mean, median, or sum.

In embodiments, the glucose impact algorithm may be implemented in software and installed as part of the meal visualization model 1306. In some embodiments, as discussed, the meal visualization model 1306 is installed on one device, for example a user device or a remote server. In other embodiments, the meal visualization model 1306 may be installed across more than one device, in which case the glucose impact algorithm may be installed in other devices within prediction framework 1642, such as in other user devices (e.g. meal visualization device 1310, user device 1302, data receiving device 120, multi-purpose data receiving device 130, user device 140, or local computer 170), in glucose monitoring devices (e.g., analyte sensor 104, sensor control device 102), and/or in backend systems such as a remote server (e.g. remote application server 155, application storefront 160, trusted computer system 180, or other remote servers accessed via cloud 190).

In embodiments, meal visualization model 1306 can map the adjusted delta to peak value to a decision-region plot to determine the predicted meal impact score, which meal visualization model 1306 can use to generate visualization elements such as score indicators 8104A-E and glucose impact component 8110B. The type of visual component comprises different colors (e.g., red/orange, yellow, green), different shapes, and different text (e.g., “minor impact,” “medium impact,” “major impact”). In embodiments, a decision-region plot can be implemented as a color-banded graph with decision regions defined over the x-axis and y-axis domains.

In embodiments, the decision-region plot can be implemented with meal peak glucose (e.g., mg/dL) reflected on the y-axis and pre-meal glucose (e.g., mg/dL) on the x-axis, and the adjusted delta to peak value can be plotted on the decision-region plot based on the provided pre-meal glucose value (i.e., the glucose value prior to the predetermined time period) and the calculated peak glucose value for the predetermined time period. In embodiments, meal visualization model 1306 may define different bands of the decision-region plot to identify various levels of predicted glucose impact. For example, adjusted delta to peak values that fall within a first band of the decision-region plot may represent a first glucose impact (e.g., major impact to a user's glucose levels), that fall within a second band a second glucose impact (e.g., a medium impact to a user's glucose levels), and that fall within a third band a third glucose impact (e.g., a minor impact to a user's glucose levels). Bands of the decision-region plot may be defined based on particular threshold values of the meal peak glucose and the pre-meal glucose.

Meal visualization model 1306 may calculate the decision-region plot based on boundary lines that define the upper and/or lower bounds for each band within the decision-region plot. In embodiments, each band can be defined as a set of coordinate pairs that satisfy linear constraints, namely a lower boundary (e.g., straight-line) and an upper boundary (e.g., straight line). The lower boundary for a band can be represented by a first linear function having a first slope, and the upper boundary can be represented by a second linear function having a second slope that may differ from the first slope. Meal visualization model 1306 can then classify values that fall within the region defined by the upper and lower bounds as associated with a particular predicted glucose impact. In some embodiments when the upper and lower bounds are linearly defined, the respective band can forms a trapezoidal or wedge-shaped region with straight edges throughout the decision-region plot.

In embodiments, meal visualization model 1306 can personalize the decision-region plot by adjusting the band regions for each user. Personalization can include adjustments to the upper and lower bounds for one or more regions within the decision-region plot such that the bands within a personalized decision-region plot may differ from user to user. For example, meal visualization model 1306 may provide different visualizations and recommendations to a first and second user having the same calculated adjusted delta to peak value if that value falls within a different band of the decision-region plot of the first user compared to the decision-region plot of the second user.

In embodiments, the glucose impact algorithm can be configured to calculate the predicted glucose impact using timing data including timestamps of user inputs (e.g., a timestamp of an image, timestamp of user input such as image upload or prompt), timestamps associated with historical glucose information, and timestamps associated with meal data. Meal visualization model 1306 can determine, using the glucose impact algorithm, a start-time of the meal based on the various timing data. Variance in the start-time time of the meal can impact the predicted glucose value (i.e., an incorrectly calculated start-time would result in an inaccurate predicted glucose value).

As noted above, meal visualization model 1306 can be further trained to perform meal concatenation when users may not provide meal information concurrently with the actual meal. For example, a user may upload all meal images for multiple meals at one time (e.g., breakfast, lunch, and dinner meal information all uploaded after dinner) or hours after the meal actually took place (e.g., uploading breakfast meal information at 2 P.M.). Another example of a discrepancy includes mismatch between a meal image timestamp and the actual time when the meal took place (e.g., a user taking a picture of a boxed meal at 3 P.M. but eating the boxed meal for dinner at 6 P.M.). Meal visualization model 1306 can be configured to confirm meal-start times agnostic to the times when the meal images and/or user inputs is received for generating the predicted glucose value.

When meal-time information is not available (e.g., not provided by the user), meal visualization model 1306 is configured to generate candidate meal-times based on a number of different available parameters including meal information (e.g., meal photos) based on user input (e.g., user providing explicit meal tag indicating breakfast, lunch, dinner, or snack), based on historical meal information (e.g., based on user history of eating pizza multiple times for breakfast), based on historical glucose information (e.g., identifying candidate meal times based on glucose information such as rises in glucose at specific times of day that are aligned with conventional breakfast, lunch, and dinner times), and/or based on input metadata, such as timestamp information associated with images and inputs. Meal visualization model 1306 can be configured to generate candidate meal-times and meal-time categorization (e.g., breakfast, lunch, or dinner), and further configured to align meal information with respective candidate meal-times.

In embodiments, meal visualization model 1306 can perform meal concatenation when one or more meals are inputted within a predetermined time window (e.g., within a one hour window, within a one to two hour range, etc.). Meal visualization model 1306 can configure the one or more inputted meal such that they are considered as part of a single meal entry. In some embodiments, when one or more inputted meals occur within a predetermined time window, the one or more inputted meal are considered as a single meal event and will be provided a singular glucose impact value. Meal visualization model 1306 can adjust the predetermined range for the time window based on historical information (e.g., prior meals and/or user preferences.

In embodiments, meal visualization model 1306 can log the multiple meals as a single meal when multiple meals are inputted within a predetermined time period of a consumed meal (e.g., within one hour of a meal). In some embodiments, the multiple meals that are logged as a single meal can comprise a timestamp of the earliest meal of the multiple inputted meals for the purposes of scoring the meal event. According to some embodiments, concatenation of meals does not extend to meals past a predetermined time within the initial meal (e.g., one hour from the initial meal).

Error check engine 1644 is configured to process outputs from meal prediction engine 1643 to ensure proper output being provided to application 1630. Examples of this process including ensuring that outputs conform to regulatory & industry guidelines by detecting wrong or unsafe output (e.g., output that provides inaccurate explanations regarding glycemic impact) and providing fallback responses for any wrong or unsafe outputs. Error check engine 1644 can be further configured to cause meal prediction engine 1643 to retry generating new output if wrong or unsafe outputs are detected.

Prompt library 1645 is configured to store and manage reusable prompt templates, i.e., the predefined prompt templates discussed elsewhere herein, for use with meal prediction engine 1643. Prompt library 1645 is communicatively coupled with meal prediction engine 1643 and can also interface with upstream components responsible for dynamically constructing model inputs, such as prompt engine 1651 or testing engine 1652, or prompt generator 1610 discussed with respect to FIG. 16A. In embodiments, each prompt template can define (i.e., have predefined) a structured prompt format that may include fixed instructions, variable placeholders for insertion of multimodal inputs, formatting constraints for outputs, and is designed to standardize input formulation and output generation for meal prediction engine 1643

Prompt library 1645 can support retrieval and instantiation of prompt templates based on multimodal input provided by prediction guardrails component 1641. During runtime, prediction framework 1642 can select and populate a prompt template with multimodal input and ensuring the generated prompt conforms to predefined structural and content requirements for meal prediction engine 1643. The resulting prompt may then be validated before being provided to meal prediction engine 1643. In embodiments, prompt library 1645 can communicate with monitoring engine 1646 by providing versioning metadata, compliance tags, or prompt lineage tracking to facilitate auditability and traceability of model behavior to specific prompt formulations. In some embodiments, the use of prompt templates can be customized and updated as additional information is provided to prediction framework 1642. For example, prompt library 1645 may provide an initial set of prompt templates tuned for a general population. Prompt template 1645 may then provide updated or personalized prompt templates for users as prediction framework 1642 collects meal information, glucose history, and other user information associated with each user.

In embodiments, prompt library 1645 can provide selection functionality for identifying and selecting prompt templates based on contextual information such as user information such as glucose data, glucose history, cohort information, and device information. For example, prompt templates can be tuned to address different contexts, including prompt templates that have been updated and personalized based on user history and interaction with prediction framework 1642. Other examples include prompt templates that are tuned for people with diabetes or people without diabetes and prompt templates for specific cohorts of people, such as a particular age group or users within a particular region, and for different glucose contexts, such as based on glucose trend information or historical data. The instructions in each prompt template can be varied to address each of the different contexts to ensure that meal prediction engine 1643 provides more accurate predictions for each user. In embodiments, prompt templates can be dynamically tuned based on user information (as discussed elsewhere herein) and the multimodal input. The prompt template may then be provided to the prompt engine 1651 or prompt generator 1610 to complete any placeholders.

In embodiments, prompt library 1645 can also store prompts for different versions of a large language model implemented for meal visualization model 1306. For example, different prompt formatting may perform better based on different types of large language models (e.g., ChatGPT, Claude, Gemini, or Llama) and/or different versions of the same large language model (e.g., ChatGPT-5, ChatGPT-4, ChatGPT-4o). Prompt library 1645 can therefore store multiple prompts optimized for different types and version of large language models. A request for a prompt from prompt library 1645 can be configured with a model identifier to indicate the model type and/or model version. Prompt library 1645 may then provide prompts based on the model identifier.

Monitoring engine 1646 is configured to be communicatively coupled to meal prediction engine 1643 and error check engine 1644 for providing audit and traceability functions to outputs from both components. Monitoring engine 1646 can be configured to monitor and log outputs generated by meal prediction engine 1643 and the corresponding results of regulatory compliance validation performed by error check engine 1644. Monitoring engine 1646 can further be configured to associate each logged output with metadata indicative of processing context, including model version, input data characteristics, regulatory criteria evaluated, and compliance outcome.

In embodiments, monitoring engine 1646 can operate in real time and in parallel with meal prediction engine 1643 and error check engine 1644, capturing output events and compliance determinations and storing as they are generated. Data captured from meal prediction engine 1643 and error check engine 1644 can be stored in a structured format that supports downstream processing for analytics, debugging, and iterative refinement of meal prediction engine 1643 and/or compliance rules associated with error check engine 1644. For instance, monitoring engine 1646 may maintain indexed logs that enable traceability of output errors to particular model states or regulatory checks.

In some embodiments, monitoring engine 1646 can includes an interface for querying historical output records and associated compliance decisions. In other embodiments, monitoring engine 1646 may trigger alerts or store anomaly indicators when output non-compliance patterns are detected over time, thereby supporting lifecycle management and validation monitoring of meal prediction engine 1643.

In embodiments, prediction framework 1642 is in communication with validation engine 1650, which is configured to test and provide validated prompts and validated human annotations for use by prediction framework 1642. Validation engine 1650 can be configured with prompt engine 1651 and testing engine 1652.

Prompt engine 1651 is configured to receive and validate prompts before they are deployed by prompt library 1645. Prompt engine 1651 can be configured with rules to validate prompts to ensure that inputs and outputs that result from the prompts conform to specified regulations and standards. In embodiments, prompt engine 1651 can be configured to modify prompt instructions so that output provided by meal prediction engine 1643 is in an expected format that conforms to the regulations and that can be further utilized by application 1630 for display of the detected meal components. Prompt engine 1651 may also have any of the functionality discussed with respect to prompt generator 1610

Testing engine 1652 is configured receive user input associated with prompts that are being validated by prompt engine 1651. User input can include annotations for confirming whether a prompt provides expected outputs (e.g., expected formatting, expected visual effects, conformance with industry standards) and feedback about whether datasets or prompts need correction or reformatting (e.g., whether datasets are mislabeled). Examples of annotations can include tagging prompts for further refinement or modification, marking datasets (e.g., low confidence, outlier), and suggesting modifications to prompt instructions. Testing engine 1652 is communicatively coupled to prompt engine 1651 to provide the user input as part of the validation process. Testing engine 1652 is also configured to store annotations and feedback for auditability and traceability functions, which can be critical for safety-critical and regulatory settings.

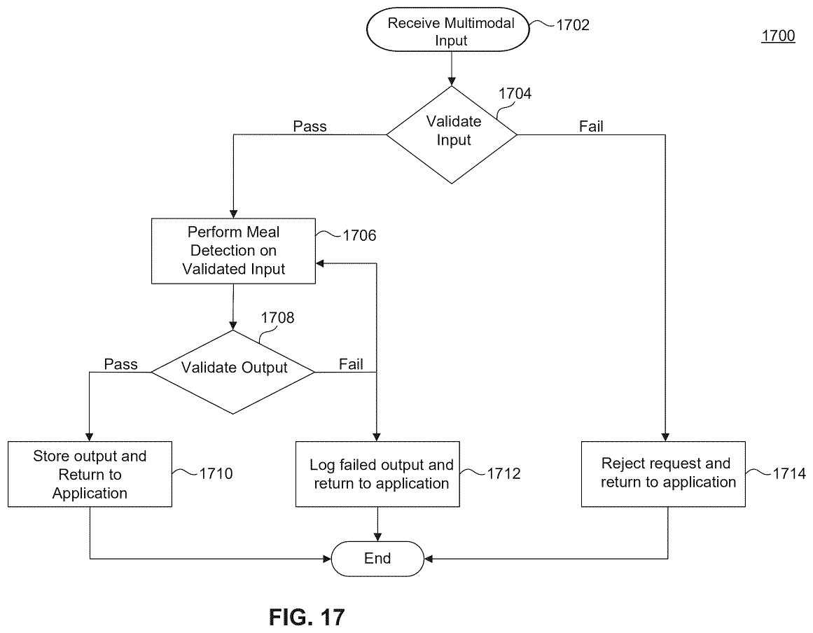

FIG. 17 is a flowchart depicting a method 1700 comprising steps for performing guardrail checks according to an embodiment. As a non-limiting example with regards to FIGS. 1-3, 13, and 16A-16B one or more processes described with respect to FIG. 17 may be performed by a meal visualization model 1306 implemented on one or more devices, including user devices (e.g., data receiving device 120 of FIG. 1, meal visualization device 1310, user device 1302, multi-purpose data receiving device 130, user device 140, and local computer 170), which may be an imaging device or a display device (e.g., meal visualization device 1310 of FIG. 13), and/or remote servers (e.g. remote application server 155, application storefront 160, trusted computer system 180, or other remote servers accessed via cloud 190). In such an embodiment, any of these devices may include suitable components (i.e., a processor) and may execute code in memory to perform certain steps of method 1700 of FIG. 17. While method 1700 of FIG. 17 will be discussed below as being performed by a meal visualization model (e.g., meal visualization model 1306) and/or a meal visualization device (e.g., meal visualization device 1310), other components may store the code and therefore may execute method 1700 by directly executing the code. Accordingly, the following discussion of method 1700 will refer to various components of FIGS. 16A and/or 16B as an exemplary non-limiting execution of method 1700. Moreover, it is to be appreciated that not all steps may be needed to perform the disclosure provided herein. Further, some of the steps may be performed simultaneously or in a different order than shown in FIG. 17, as will be understood by a person of ordinary skill in the art. Moreover, various steps of FIG. 17 may be performed with other steps of the disclosure, including but not limited to the steps of FIG. 14 and/or FIG. 15.

In 1702, meal visualization model 1306 receives multimodal input from application 1630. The multimodal input may take the form as described with respect to multimodal input 1632 of FIG. 16B, which in turn makes reference to image data, additional user input and, in some embodiments, additional data (i.e. as described with respect to the method of FIG. 15). In embodiments, meal visualization model 1306 is configured to identify the type of multimodal input, such as text, image, video, and/or audio inputs. In embodiments, meal visualization model 1306 is configured with one or more components for processing different types of multimodal input.

In 1704, meal visualization model 1306 validates the multimodal input. Validation of multimodal input can include different steps for determining that the multimodal input does not include improper or inappropriate input (e.g., non-meal related input, unsafe or dangerous input, faces, sensitive private information). Meal visualization model 1306 may be configured with one or more components for identifying inappropriate content in the multimodal input. In embodiments, the components can include predefined rules identifying inappropriate content or a trained model for dynamically identifying inappropriate content in multimedia and text.

In 1706, meal visualization model 1306 performs meal detection on the validated input. Meal detection includes steps for selecting a prompt template (e.g., from prompt library 1645), forming a prompt based on the selected prompt template and the validated multimodal input, and generating output comprising any detected meal components in the multimodal input. The output may include meal textual information discussed elsewhere herein, whilst the detected meal components are an aspect of meal classification information. Format and the type of output generated by meal visualization model 1306 can be based on both how meal visualization model 1306 other than meal textual information is trained and the output instructions included in the selected prompt template. Output format may be dictated by metadata in the meal textual information, as discussed elsewhere herein. Examples of output format include how detected meal objects are organized, including text description of the detected meal objects, a predicted glycemic impact of each of the detected meal objects, an overall predicted glycemic impact of the combined detected meal objects, recommendation text associated with one or more of the detected meal objects, color coding associated with each detected meal object (e.g., color coding corresponding to a predicted glycemic impact), and other instructions defining how the detected meal objects are visually represented by application 1630. Other possible outputs include those to generate any of the graphical user interfaces shown in FIGS. 8A-12B. In some embodiments, the impact of the recommendation may also be determined in this step.

In 1708, meal visualization model 1306 can validate its output, including the detected meal components (i.e., meal classification information) and the output format for each of the detected meal components. In embodiments, meal visualization model 1306 may provide the output to one or more guardrail components for validating the output. This step ensures that output from meal visualization model 1306 conforms with one or more organizational or industry-specific safety, regulatory, and operational requirements. This step can include a combination of automated (e.g., guardrail components) and/or human-in-the-loop review of AI outputs (i.e. outputs from the meal prediction engine 1643) to confirm that such outputs satisfy applicable safety standards, such as those governing clinical decision support tools, medical device software, or patient communication protocols, conform to regulatory frameworks established by relevant authorities (e.g., FDA, HIPAA, GDPR) or internal company policies governing the permissible use and disclosure of sensitive data.

In embodiments, validating the output can include removal of specific terms, such as trademark names and brands. Validating output can include masking (or generalizing) or replacing identified trademark names and brands with predefined generic terms, such as converting a detected brand name with an associated generic term.

Validation of output can include ensuring accuracy and factual integrity of the output, ensuring that the text output aligns with predefined brand tone, voice, and messaging guidelines, and avoids unsafe or misleading recommendations, such as by cross-referencing the output with known constraints, clinical rules, or validated decision support pathways. In some embodiments, meal visualization model 1306 can employ any combination of rule-based checks, natural language processing (NLP) classifiers, safety filters, domain-specific knowledge graphs, or external validation APIs as part of the validation process. In embodiments, if output fails validation, meal visualization model 1306 may repeat 1706 to generate new output.

In embodiments, the output includes instructions (i.e., meal textual information) for displaying the detected meal components (i.e., meal classification information) along with any other related information including recommendation text, a predicted glycemic impact of the meal components and/or meal, and any visual indicators such as colors or text.

In 1710, meal visualization model 1306 stores validated output (e.g., in monitoring engine 1646) and returns the output for displaying the detected meal components and any associated visual indicators and information by the application 1630.

In 1712, meal visualization model 1306 logs any output that fails validation and returns an error message to the application 1630.

In 1714, meal visualization model 1306 rejects the multimodal input if the validation fails and returns an error message to the application 1630.

It is to be appreciated that the Summary and Abstract sections may set forth one or more but not all exemplary embodiments of the present invention as contemplated by the inventor(s), and thus, are not intended to limit the present invention and the appended claims in any way.

The foregoing description of the system and methods will so fully reveal the general nature of the invention that others can, by applying knowledge within the skill of the art, readily modify and/or adapt for various applications the systems and methods, without undue experimentation, without departing from the general concept of the present invention. Therefore, such adaptations and modifications are intended to be within the meaning and range of equivalents of the disclosed invention, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described examples, but should be defined only in accordance with the following claims and their equivalents. To the extent that any processor-implemented method are referred to herein (particularly the methods of FIG. 14, FIG. 15, FIG. 16A and/or FIG. 17), the present disclosure extends to computer-implemented methods and computer-readable storage media (including non-transitory computer readable storage media) configured to cause one or more processors (from one or more devices as discussed herein) to perform the methods of the invention.

### Glossary

The following definitions are not intended to be overly restrictive. Interpretation of the application is ultimately a matter for a person skilled in the art. These definitions are provided to assist in construing the claims. In particular, where a definition is given, it may guide the meaning of the term within this specification unless the context clearly dictates otherwise.

The terms “multimodal” and “multi-modal” as used herein refer to two or more modes of data. For data, mode is a distinct form or type of representation. For example, image data and additional user input are two different modes of data. Other examples of modes include other forms of media such as textual data (i.e. text data), audio data, video data, additional image data. Further examples of modes include medical history, query data, (continuous) analyte data. Different modes may be from the same or different sources.

The term “meal” as used herein is an eating occasion that involves one or more meal components (also referred to herein as meal objects). Meals may be categorized by type, such as breakfast, lunch, dinner, snack, and the like. Meals may have attributes such as portion size, nutritional content, ingredients, and the like. Meal attributes are the sum of meal component attributes.

The terms “meal object” and “meal component” refer to a distinct type of food or beverage. For example, white rice, although consisting of many individual rice gains, is a single meal component. Meal components may have attributes such as portion size, nutritional content, ingredients, and the like.

The term “meal visualization model”, as used herein encompasses any models and algorithms described herein that manipulate data, particularly multimodal data, for the purpose of providing an output to a display or display device. The output typically takes the form of a meal visualization interface (i.e. graphical user interface). In general, the meal visualization model may include at least a portion which is implemented with AI, and thus can be referred to as a trained model. In some embodiments, the meal visualization model may be or include a pre-trained or pre-existing model, particularly a large language model, and especially a multimodal large language model (e.g. ChatGPT-4 and similar). In some embodiments, the meal visualization model may include several sub-models to enable it to handle multimodal data.

The terms “meal visualization system” and “meal visualization device” encompasses any system or device described herein capable of generating a meal visualization interface (i.e. graphical user interface). A particular example of a meal visualization system and meal visualization device are described in FIG. 13. In some embodiments, the meal visualization system and meal visualization device may incorporate the imaging device, user interface and/or the display (also referred to as the display device). In particular embodiments, the imaging device, user interface and/or display may be the meal visualization device. Alternatively, the meal visualization system and meal visualization device may be distinct from the imaging device, user interface and/or the display (also referred to as the display device). Irrespective of whether the imaging device, user interface and/or the display forms part of the meal visualization system or not, the meal visualization system is in communication (i.e. communicatively coupled) with the imaging device, user interface and/or the display.

The term “imaging device” as used herein is a reference to any device herein capable of capturing image data, typically via a camera, including an integrated camera. Imaging devices described in the specification include user devices such as meal visualization device 1310, user device 1302, data receiving device 120, multi-purpose data receiving device 130, user device 140, and local computer 170.

The terms “user interface” and “graphical user interface” are references to an interface, particularly a graphical user interface, provided to a user by a user device. User devices described in the specification include meal visualization device 1310, user device 1302, data receiving device 120, multi-purpose data receiving device 130, user device 140, or local computer 170. In many instances, the user interface is displayed by a touch screen display so that the user can interact with interface components of the graphical user interface.

The terms “display” and “display device”, as used herein, are references to any device herein capable of displaying data to a user. In many instances, the display or display device may be a touch screen so that the user can interact with interface components of graphical user interfaces. Display devices described in the specification include meal visualization device 1310, user device 1302, data receiving device 120, multi-purpose data receiving device 130, user device 140, and local computer 170.

The term “continuous analyte monitor” and variations thereof including “analyte monitoring system”, “in vivo analyte monitoring system”, “glucose monitoring device”, “glucose monitoring systems”, “glucose sensor”, “analyte sensor” as used herein, are devices or systems for measuring analyte (particularly glucose) continuously. Particular examples of such systems and devices are analyte sensor 104 described with respect to FIG. 1B and/or the sensor control device 102 described with respect to FIG. 1A.

The terms “analyte data”, “analyte level”, “glucose data”, “glucose level” and related variations (including the adjective “historic”, “historical”, “measured”, and similar) all reference measurements taken of the user by the continuous analyte monitor, particularly of the user's glucose levels.

The terms “glycemic impact value”, “glycemic impact score”, “glycemic impact”, “glucose impact score”, “glucose impact value”, “glucose impact” refer to an impact on glucose of consuming a meal. In some instances, this impact may be predicted, i.e. “predicted glycemic impact” or “glycemic impact prediction” as used herein. In other instances, the impact may be the actual impact, i.e. “actual glycemic impact.”

### Numbered Clauses

The following is a numbered list of clauses of the invention. The clauses may be combined according to any combination.

Clause 1: A method for meal visualization comprising:
receiving image data from an imaging device;
identifying a first placeholder location in a predefined prompt template;
   generating a meal prompt by inserting the image data in the first placeholder location;
   providing the meal prompt as input to a meal visualization model;
   receive, from the meal visualization model and responsive to providing the meal prompt, meal classification information and meal textual information, wherein the meal classification information comprises a first meal identifier associated with a first meal object detected in the image data by the meal visualization model and a second meal identifier associated with a second meal object detected in the image data by the meal visualization model, and wherein the meal textual information is generated by the meal visualization model based on the meal classification information; and
   displaying, on a display, a meal visualization interface comprising a first interface component and a second interface component, wherein the first interface component comprises a first graphical element that displays the first meal identifier and a second graphical element that displays the second meal identifier, and the second interface component displays at least a portion of the meal textual information.

Clause 2: The method of clause 1, wherein the method is performed by one or more processors.

Clause 3: The method of clause 1 or 2, wherein the method is performed by a meal visualization system or a meal visualization device.

Clause 4: The method of clause 3, wherein the meal visualization system or the meal visualization device is in communication with the imaging device and a user interface.

Clause 5: The method of clause 3 or 4, wherein the meal visualization system or the meal visualization device comprises the display.

Clause 6: The method of any one of clauses 3-5, wherein the meal visualization system or the meal visualization device comprises a remote server and/or a user device.

Clause 7: The method of any one of clauses 3-6, wherein the meal visualization system further comprises a continuous analyte monitor, wherein the continuous analyte monitor is configured to generate analyte data based on a measured analyte level.

Clause 8: The method of any one of clauses 3-7, wherein the meal visualization system further comprises an insulin delivery device.

Clause 9: The method of any one of clauses 3-8, wherein the meal visualization system further comprises one or more sensors.

Clause 10: The method of any one of clauses 3-9, wherein the one or more sensors are comprised in a wearable device.

Clause 11: The method of any one of clauses 3-10, wherein the meal visualization system or the meal visualization device is in communication with one or more external databases.

Clause 12: The method of clause 11, wherein the one or more external databases comprise a food database, a recipe database, or a nutrition database.

Clause 13: The method of clause 11 or 12, wherein the one or more external databases comprise electronic health records.

Clause 14: The method of any preceding clause, wherein the imaging device comprises the user interface and/or the display.

Clause 15: The method of any preceding clause, wherein the imaging device comprises an integrated camera.

Clause 16: The method of any preceding clause, wherein the imaging device is a user device.

Clause 17: The method of any preceding clause, wherein the user interface is a graphical user interface.

Clause 18: The method of any preceding clause, wherein the display is a touch screen.

Clause 19: The method of any preceding clause, wherein receiving image data from the imaging device comprises receiving the image data from a captured image from an image library of the imaging device.

Clause 20: The method of any preceding clause, wherein receiving image data from the imaging device comprises receiving the image data from an image and/or video capture feature of the imaging device.

Clause 21: The method of any preceding clause, wherein the image data relates to a meal.

Clause 22: The method of clause 21, wherein the image data comprises an image of a menu, food and/or beverage.

Clause 23: The method of any preceding clause, wherein the image data comprises an image of at least the first meal object and the second meal object.

Clause 24: The method of any preceding clause, wherein the image data comprises visual data depicting at least the first meal object and the second meal object.

Clause 25: The method of clause 24, wherein the meal visualization model performs image recognition on the image data to detect the first meal object and the second meal object.

Clause 26: The method of any preceding clause, wherein the image data comprises textual data.

Clause 27: The method of clause 26, wherein the textual data describes at least the first meal object and the second meal object, Clause 28: The method of clause 27, wherein the meal visualization model performs text recognition on the image data to detect the first meal object and the second meal object and wherein each of the first meal object and the second meal object is a distinct type of food or beverage.

Clause 29: The method of any preceding clause, further comprising: receiving metadata of the image data from the imaging device.

Clause 30: The method of any preceding clause, further comprising:
receiving additional user input from a user interface; and
identifying a second placeholder location in the predefined prompt template,
wherein generating the meal prompt further comprises inserting the additional user input into the second placeholder location

Clause 31: The method of any preceding clause, wherein receiving the additional user input from the user interface comprises:
storing the additional user input in a memory; and
retrieving the additional user input from the memory.

Clause 32: The method of any preceding clause, wherein the additional user input comprises or relates to data from a user device, a remote server, a continuous analyte monitor, an insulin delivery device, one or more sensors, or one or more external databases.

Clause 33: The method of any preceding clause, wherein the additional user input comprises user medical history, optionally, wherein the user medical history comprises user analyte history.

Clause 34: The method of clause 33, wherein the user medical history is obtained from a continuous analyte monitor or a remote server.

Clause 35: The method of any one of clauses 30-34, wherein the additional user input comprises user meal history.

Clause 36: The method of any one of clauses 30-35, wherein the additional user input comprises at least one of textual data, audio data, video data, or additional image data.

Clause 37: The method of any one of clauses 30-36, wherein the additional user input comprises meal information associated with the image data, optionally, wherein the meal information comprises nutritional data associated with at least one of the first meal object and the second meal object.

Clause 38: The method of clause 37, wherein the meal information comprises meal type associated with at least one of the first meal object and the second meal object.

Clause 39: The method of any one of clauses 37-38, wherein the meal information comprises a portion size associated with at least one of the first meal object and the second meal object.

Clause 40: The method of any one of clauses 30-39, wherein the additional user input comprises query data.

Clause 41: The method of clause 40, wherein the query data is input via a chat interface component of the user interface.

Clause 42: The method of clause 40 or 41, wherein the query data comprises an initial query and a follow up query.

Clause 43: The method of any one of clauses 30-42, wherein the additional user input comprises analyte data.

Clause 44: The method of clause 43, wherein the analyte data comprises a current glucose value.

Clause 45: The method of clause 43 or 44, wherein the analyte data is obtained from a continuous glucose monitor.

Clause 46: The method of any one of clauses 30-45, wherein the additional user input comprises a user correction or user feedback.

Clause 47: The method of any one of clauses 30-46, wherein the additional user input comprises user information.

Clause 48: The method of clause 47, wherein the user information comprises meal preferences.

Clause 49: The method of clause 48, wherein the meal preferences comprise diabetes diagnosis, optionally wherein the diabetes diagnosis is one of type 2 diabetes, type 1 diabetes, and pre-diabetes.

Clause 50: The method of clause 48 or 49, wherein the meal preferences comprise diabetes management, optionally wherein the diabetes management is one or more of diet, exercise, insulin pump, glucagon-like peptide (GLP)-1, long-acting or basal insulin, non-insulin injectables, oral medications (tablets or pills), short or rapid acting insulin, and bolus insulin.

Clause 51: The method of any one of clauses 48-50, wherein the meal preferences comprise dietary preferences, optionally wherein the dietary preferences include all food, vegetarian, vegan, pescatarian, keto, paleo, gluten-free, and dairy-free.

Clause 52: The method of any one of clauses 48-51, wherein the meal preferences comprise dietary restrictions, optionally wherein the dietary preferences include any allergies including peanuts, tree nuts, milk, eggs, fish, shellfish, soy, wheat, and mushrooms.

Clause 53: The method of any preceding clause, further comprising: receiving additional data from a user device, a remote server, a continuous analyte monitor, an insulin delivery device, one or more sensors, or one or more external databases.

Clause 54: The method of any preceding clause, wherein identifying the first placeholder location in the predefined prompt template is based on data type, and wherein when dependent on clause 30, identifying the second placeholder location in the predefined prompt template is based on data type.

Clause 55: The method of any preceding clause, wherein the predefined prompt template is obtained from a prompt library.

Clause 56: The method of clause 55, wherein the prompt library contains a plurality of predefined prompt templates, further comprising selecting a prompt template from the plurality of predefined prompt templates based on user information.

Clause 57: The method of any preceding clause, wherein the predefined prompt template is generalized and wherein the predefined prompt template is personalized based on user information.

Clause 58: The method of any preceding clause, wherein the predefined prompt template comprises or points to an algorithm for determining glycemic impact score.

Clause 59: The method of any preceding clause, wherein the predefined prompt template comprises or points to an algorithm for determining insulin dose recommendation.

Clause 60: The method of any preceding clause, wherein the predefined prompt template comprises instructions to cause the meal visualization model to provide output as a structured output file.

Clause 61: The method of any preceding clause, wherein the predefined prompt template comprises instructions to cause the meal visualization model to output one or more of: a confidence score associated with each meal object, a glycemic impact score associated with each meal object, an overall glycemic impact score associated with the meal, and recommendations based on the meal.

Clause 62: The method of any preceding clause, wherein the predefined prompt template defines output rules for the interface components.

Clause 63: The method of clause 62, wherein the output rules comprise one or more of: format, layout, position, visual component and not outputting trademarked names or brands.

Clause 64: The method of any preceding clause, wherein the meal prompt is generated by a prompt generator or prompt engine.

Clause 65: The method of any preceding clause, wherein the meal visualization model comprises a pre-trained or pre-existing machine learning model.

Clause 66: The method of any preceding clause, wherein the meal visualization model comprises a large language model.

Clause 67: The method of any preceding clause, wherein the meal visualization model comprises a multimodal large language model.

Clause 68: The method of any preceding clause, wherein the meal visualization model comprises one or more sub-models.

Clause 69: The method of clause 68, wherein the one or more sub-models comprise: a media recognition component, a natural language component, a media output component, a knowledge retrieval component, and a file interpretation component.

Clause 70: The method of any preceding clause, wherein the meal visualization model comprises one or more of: a prediction guardrails component, an error check engine, a prompt library, and a monitoring engine.

Clause 71: The method of any preceding clause, wherein the meal visualization model is in communication with a validation engine, optionally wherein the validation engine comprises a prompt engine and/or a testing engine.

Clause 72: The method of any preceding clause, wherein the meal visualization model outputs a structured output file.

Clause 73: The method of any preceding clause, further comprising:
determining, by the meal visualization model, an intent of query data;
generating the meal textual information based on the query data,

Clause 74: The method of clause 73, wherein generating the meal textual information is also based on analyte data, and/or image data, wherein the meal textual information is personalized based on the query data and/or the analyte data.

Clause 75: The method of any preceding clause, wherein meal classification information further comprises meal type and/or meal object type.

Clause 76: The method of any preceding clause, wherein the meal classification information further comprises a first color-coded tag.

Clause 77: The method of clause 76, wherein the first color-coded tag is associated with a glycemic impact score of the meal.

Clause 78: The method of clause 76, wherein the meal classification information further comprises a second color-coded tag.

Clause 79: The method of clause 78, wherein the first meal identifier is associated with a first glucose impact value and the second meal identifier is associated with a second glucose impact value, wherein the first color-coded tag is determined based on the first glycemic impact score and the second color-coded tag is determined based on the second glycemic impact score,

Clause 80: The method of clause 79, wherein the first graphical element is displayed based on the first color-coded tag, and the second graphical element is displayed based on the second color-coded tag.

Clause 81: The method of any preceding clause, wherein the meal classification information is determined by the meal visualization model taking into account one or more meal preferences of the user.

Clause 82: The method of any preceding clause, further comprising:
obtaining, by the meal visualization model, first meal object metadata and second meal object metadata based on the first meal identifier and the second meal identifier.

Clause 83: The method of clause 82, wherein the first meal object metadata and the second meal object metadata includes any one of the format, layout, position, visual component, that is associated with presenting the meal object.

Clause 84: The method of clause 82 or 83, wherein the first meal object metadata and the second meal object metadata is provided as defined by output rules in the predefined prompt template.

Clause 85: The method of clause 82 or 83, wherein the first meal object metadata and the second meal object metadata is provided via a look-up table based on the meal object identifier.

Clause 86: The method of any preceding clause, wherein the meal textual information comprises a glycemic impact score.

Clause 87: The method of clause 86, wherein the meal textual information further comprises metadata for determining visualization of the glycemic impact score.

Clause 88: The method of clause 87, wherein the metadata is provided as defined by output rules in the predefined prompt template.

Clause 89: The method of clause 86, wherein the metadata is obtained via a look-up table based on the glycemic impact prediction.

Clause 90: The method of any one of clauses 87-89, wherein the metadata comprises any one of the format, layout, position, visual component, that is associated with presenting the glycemic impact score.

Clause 91: The method of any one of clauses 86-90, wherein the glycemic impact score is a generalized glycemic impact score.

Clause 92: The method of any one of clauses 86-90, wherein the glycemic impact score comprises a first glycemic impact score for the first meal object and a second glycemic impact score for the second meal object.

Clause 93: The method of any one of clauses 86-92, wherein the glycemic impact score is a personalized glycemic impact score.

Clause 94: The method of any preceding clause, wherein the meal textual information comprises one or more of: summary text, nutrition text, and narrative text.

Clause 95: The method of any preceding clause, wherein the meal textual information comprises a meal recommendation.

Clause 96: The method of clause 95, wherein the meal recommendation is dynamic based on user information.

Clause 97: The method of any preceding clause, wherein the first graphical element and the second graphical element are color coded.

Clause 98: The method of any preceding clause, wherein the first graphical element and the second graphical element have a score indicator to indicate glycemic impact score.

Clause 99: The method of clause 98, wherein the glycemic impact score may be a generalized glycemic impact score or a personalized glycemic impact score.

Clause 100: The method of any preceding clause, wherein the meal visualization interface further comprises a third interface component, wherein the third interface component is configured to receive real-time textual queries.

Clause 101: The method of clause 100, further comprising:
receiving, via the third interface component, a real-time textual query;
providing the real-time textual query as an additional input to the meal visualization model;
receiving, from the meal visualization model and responsive to providing the real-time textual query, responsive analysis information; and
updating a fourth interface component of the meal visualization interface to display the responsive analysis information.

Clause 102: The method of clause 101, further comprising:
receiving analyte data from a continuous analyte monitor;
providing the analyte data as an additional input to the meal visualization model;
wherein the responsive analysis information is also responsive to the analyte data.

Clause 103: The method of clause 102 or 103, further comprising:
determining, by the meal visualization model, the intent of the real-time textual query,
wherein the responsive analysis information is based on the intent.

Clause 104: The method of any one of clauses 100-103, wherein the responsive analysis information comprises a meal recommendation.

Clause 105: The method of clause 104, wherein the meal recommendation comprises nutritional information, a meal object identifier, and a text-based justification.

Clause 106: The method of any preceding clause, wherein the meal visualization interface comprises a third interface component and wherein the method further comprises:
receiving, from the meal visualization model, a generalized glycemic impact score based on the meal classification information; and
updating the third interface component to visually represent the generalized glycemic impact score.

Clause 107: The method of clause 106, wherein updating the third interface component to visually represent the generalized glycemic impact score comprises color coding.

Clause 108: The method of clause 106 or 107, wherein the generalized glycemic impact score is divided into different regions depicting different glycemic impact thresholds.

Clause 109: The method of any one of clauses 106-108, wherein the generalized glycemic impact score visually represents the glycemic impact value for a selected period of time.

Clause 110: The method of any one of clauses 106-109, wherein the generalized glycemic impact score is dynamically configurable and updateable to illustrate glycemic impact score over a predefined period of time.

Clause 111: The method of any one of clauses 106-110, wherein the generalized glycemic impact score is for the first meal object or the second meal object.

Clause 112: The method of any one of clauses 106-110, wherein the generalized glycemic impact score is for the first meal object and the second meal object.

Clause 113: The method of any one of clauses 106-111, wherein the generalized glycemic impact score is based on analyte data.

Clause 114: The method of clause 113, wherein the analyte data comprises current glucose level and/or historical glucose data.

Clause 115: The method of any one of clauses 106-114, further comprising:
receiving analyte data from a continuous glucose monitor,
updating the third component to visually represent the actual glycemic impact score.

Clause 116: The method of any one of clauses 106-115, further comprising:
updating the generalized glycemic impact score based on the user medical history to generate a personalized glycemic impact score; and
updating the third interface component to visually represent the personalized glycemic impact score.

Clause 117: The method of any one of clauses 106-116, further comprising:
receiving a user selection of a selectable object of meal visualization interface, wherein the user selection is associated with a recommended change to the meal classification information; and
updating the third interface component to display an updated generalized or personalized glycemic impact score without transmitting the user selection to the meal visualization model.

Clause 118: The method of clause 117, wherein the recommended change to the meal classification information comprises one or more proposed modifications to the first and second meal objects.

Clause 119: The method of clause 118, wherein the recommended change is to lower the generalized or personalized glycemic impact score.

Clause 120: The method of any one of clauses 117-119, wherein updating the third interface is performed using a structured output file.

Clause 121: The method of any preceding clause, further comprising:
updating the meal visualization interface to display a user input component.

Clause 122: The method of clause 121, further comprising:
receiving, via the user input component, additional meal classification information;
providing the additional meal classification information to the meal visualization model, wherein the meal visualization model is configured to identify a third meal identifier based on the additional meal classification information.

Clause 123: The method of clause 122, further comprising: updating the first interface component to display a third graphical element in proximity to the first graphical element and the second graphical element, wherein the third graphical element is configured to display the third meal identifier.

Clause 124: The method of clause 123, further comprising:
receiving, from the meal visualization model and responsive to providing the additional meal classification information, meal classification information,
wherein the third meal identifier is associated with a third glucose impact value, wherein the graphical meal classification information further comprises a third color-coded tag, wherein the third color-coded tag is determined based on the third glucose impact value, and wherein the third graphical element is displayed based on the third color-coded tag.

Clause 125: The method of any preceding clause, wherein the meal visualization interface comprises a third interface component, wherein the meal visualization model is configured to provide the meal classification information and the meal textual information in a structured output file.

Clause 126: The method of clause 125, wherein the structured output file further comprises a predicted glycemic impact value, the method further comprising:
retrieving dose recommendation information based on the predicted glycemic impact value from the structured output file; and
displaying the dose recommendation information as part of the third interface component.

Clause 127: The method of clause 126, wherein the dose recommendation information further comprises layout data generated based on instructions in the predefined dose template, wherein displaying the dose recommendation information as part of the third interface component is based on the layout data.

Clause 128: A meal visualization system comprising one or more processors and a display, the meal visualization system configured to perform the method of any preceding clause.

Clause 129: A computer readable medium comprising instructions which, when executed by one or more processors of a meal visualization system with a display, cause the meal visualization system to perform the method of any preceding clause.

Clause 130: A method for providing dose recommendations for meals with an imaging device configured to display a user interface, the method comprising:
receiving image data from the imaging device;
receiving additional user input from the user interface;
identifying a first placeholder location and a second placeholder location in a predefined prompt template;
generating a meal prompt by inserting the image data in the first placeholder location and the additional user input into the second placeholder location;
providing the meal prompt as input to a meal visualization model;
receiving, from the meal visualization model and responsive to providing the meal prompt, meal classification information and meal textual information, wherein the meal classification information comprises a first meal identifier associated with a first meal object detected in the image data by the meal visualization model and a second meal identifier associated with a second meal object detected in the image data by the meal visualization model, and wherein the meal textual information is generated by the meal visualization model based on the meal classification information; and
displaying, on a display of the meal visualization system, a meal visualization interface comprising a first interface component and a second interface component, wherein the first interface component comprises a first graphical element configured to display the first meal identifier and a second graphical element configured to display the second meal identifier, and the second interface component is configured to display the meal textual information.

Clause 131: A meal visualization system in communication with an imaging device and a user interface, the meal visualization system configured to:
receive image data from the imaging device;
receive additional user input from the user interface;
provide the image data and additional user input as input to a meal visualization model;
receive, from the meal visualization model and responsive to providing the image data and the additional user input, meal classification information and meal textual information, wherein the meal classification information comprises a first meal identifier associated with a first meal object detected in the image data by the meal visualization model and a second meal identifier associated with a second meal object detected in the image data by the meal visualization model, and wherein the meal textual information is generated by the meal visualization model based on the meal classification information; and
display, on a display of the meal visualization system, a meal visualization interface comprising a first interface component and a second interface component, wherein the first interface component comprises a first graphical element configured to display the first meal identifier and a second graphical element configured to display the second meal identifier, and the second interface component is configured to display at least a portion of the meal textual information.

What is claimed is:

1. A method for meal visualization comprising:
receiving image data from an imaging device;
identifying a first placeholder location in a predefined prompt template;
generating a meal prompt by inserting the image data in the first placeholder location;
providing the meal prompt as input to a meal visualization model;
receive, from the meal visualization model and responsive to providing the meal prompt, meal classification information and meal textual information, wherein the meal classification information comprises a first meal identifier associated with a first meal object detected in the image data by the meal visualization model and a second meal identifier associated with a second meal object detected in the image data by the meal visualization model, and wherein the meal textual information is generated by the meal visualization model based on the meal classification information; and
displaying, on a display, a meal visualization interface comprising a first interface component and a second interface component, wherein the first interface component comprises a first graphical element that displays the first meal identifier and a second graphical element that displays the second meal identifier, and the second interface component displays at least a portion of the meal textual information.

2. The method of claim 1, wherein the method is performed by one or more processors.

3. The method of claim 1, wherein the method is performed by a meal visualization system or a meal visualization device.

4. The method of claim 3, wherein the meal visualization system or the meal visualization device is in communication with the imaging device and a user interface.

5. The method of claim 3, wherein the meal visualization system or the meal visualization device comprises the display.

6. The method of claim 3, wherein the meal visualization system or the meal visualization device comprises a remote server and/or a user device.

7. The method of claim 3, wherein the meal visualization system further comprises a continuous analyte monitor, wherein the continuous analyte monitor is configured to generate analyte data based on a measured analyte level.

8. The method of claim 3, wherein the meal visualization system further comprises an insulin delivery device.

9. The method of claim 3, wherein the meal visualization system further comprises one or more sensors.

10. The method of claim 3, wherein the one or more sensors are comprised in a wearable device.

11. The method claim 3, wherein the meal visualization system or the meal visualization device is in communication with one or more external databases.

12. The method of claim 11, wherein the one or more external databases comprise a food database, a recipe database, or a nutrition database.

13. The method of claim 11, wherein the one or more external databases comprise electronic health records.

14. The method of claim 1, wherein the imaging device comprises the user interface and/or the display.

15. The method of claim 1, wherein the imaging device comprises an integrated camera.

16. The method of claim 1, wherein the imaging device is a user device.

17. The method of claim 1, wherein the user interface is a graphical user interface.

18. The method of claim 1, wherein the display is a touch screen.

19. The method of claim 1, wherein receiving image data from the imaging device comprises receiving the image data from a captured image from an image library of the imaging device.

20. The method of claim 1, wherein receiving image data from the imaging device comprises receiving the image data from an image and/or video capture feature of the imaging device.
